SEQUENCE LISTING (43 sequences)

SEQ ID NO: 1 - DNA, 20 bp, Artificial Sequence
  Synthetic primer m18S-F
tcaagaacgaaagtcggagg

SEQ ID NO: 2 - DNA, 20 bp, Artificial Sequence
  Synthetic primer m18S-R
ggacatctaagggcatcaca

SEQ ID NO: 3 - DNA, 19 bp, Artificial Sequence
  Synthetic primer mCdkn1a-F
gtggccttgtcgctgtctt

SEQ ID NO: 4 - DNA, 23 bp, Artificial Sequence
  Synthetic primer mCdkn1a-R
gcgcttggagtgatagaaatctg

SEQ ID NO: 5 - DNA, 20 bp, Artificial Sequence
  Synthetic primer mCdkn1c-F
cgaggagcaggacgagaatc

SEQ ID NO: 6 - DNA, 21 bp, Artificial Sequence
  Synthetic primer mCdkn1c-R
gaagaagtcgttcgcattggc

SEQ ID NO: 7 - DNA, 21 bp, Artificial Sequence
  Synthetic primer mCnp-F
tttacccgcaaaagccacaca

SEQ ID NO: 8 - DNA, 21 bp, Artificial Sequence
  Synthetic primer mCnp-R
caccgtgtcctcatcttgaag

SEQ ID NO: 9 - DNA, 22 bp, Artificial Sequence
  Synthetic primer mGfap-F
ccaagatgaaaccaacctgagg

SEQ ID NO: 10 - DNA, 22 bp, Artificial Sequence
  Synthetic primer mGfap-R
tctctccaaatccacacgagcc

SEQ ID NO: 11 - DNA, 20 bp, Artificial Sequence
  Synthetic primer mId1-F
cctagctgttcgctgaaggc

SEQ ID NO: 12 - DNA, 21 bp, Artificial Sequence
  Synthetic primer mId1-R
ctccgacagaccaagtaccac

SEQ ID NO: 13 - DNA, 21 bp, Artificial Sequence
  Synthetic primer mId2-F
atgaaagccttcagtccggtg

SEQ ID NO: 14 - DNA, 19 bp, Artificial Sequence
  Synthetic primer mId2-F
agcagactcatcgggtcgt

SEQ ID NO: 15 - DNA, 21 bp, Artificial Sequence
  Synthetic primer mId3-F
tgtcgtccaagaggctaagag

SEQ ID NO: 16 - DNA, 20 bp, Artificial Sequence
  Synthetic primer mId3-R
tgctacgaggcggtgtgctg

SEQ ID NO: 17 - DNA, 20 bp, Artificial Sequence
  Synthetic primer mMbp-F
gggcatccttgactccatcg

SEQ ID NO: 18 - DNA, 18 bp, Artificial Sequence
  Synthetic primer mMbp-R
gctctgctttagccaggg

SEQ ID NO: 19 - DNA, 20 bp, Artificial Sequence
  Synthetic primer mNestin-F
gagctggagcgcgagttaga

SEQ ID NO: 20 - DNA, 20 bp, Artificial Sequence
  Synthetic primer mNestin-R
gccacttccagactaaggga

SEQ ID NO: 21 - DNA, 19 bp, Artificial Sequence
  Synthetic primer mNg2-F
gggctgtgctgtctgttga

SEQ ID NO: 22 - DNA, 21 bp, Artificial Sequence
  Synthetic primer mNg2-R
tgattcccttcaggtaaggca

SEQ ID NO: 23 - DNA, 21 bp, Artificial Sequence
  Synthetic primer mNkx2.2-F
aagcatttcaaaaccgacgga

SEQ ID NO: 24 - DNA, 23 bp, Artificial Sequence
  Synthetic primer mNkx2.2-R
cctcaaatccacagatgaccaga

SEQ ID NO: 25 - DNA, 20 bp, Artificial Sequence
  Synthetic primer mOlig1-F
tcttccaccgcatcccttct

SEQ ID NO: 26 - DNA, 23 bp, Artificial Sequence
  Synthetic primer mOlig1-R
ccgagtagggtaggataacttcg

SEQ ID NO: 27 - DNA, 22 bp, Artificial Sequence
  Synthetic primer mPdgfa-F
tccatgctagactcagaagtca

SEQ ID NO: 28 - DNA, 21 bp, Artificial Sequence
  Synthetic primer mPdgfa-R
tcccggtggacacaatttttc

SEQ ID NO: 29 - DNA, 23 bp, Artificial Sequence
  Synthetic primer mRap1gap-F
ctcgctcaagtatgatgtcatcg

SEQ ID NO: 30 - DNA, 21 bp, Artificial Sequence
  Synthetic primer mRap1gap-R
ggtaaagcacggggtagaatc

SEQ ID NO: 31 - DNA, 21 bp, Artificial Sequence
  Synthetic primer mSox10-F
acaccttgggacacggttttc

SEQ ID NO: 32 - DNA, 21 bp, Artificial Sequence
  Synthetic primer mSox10-R
taggtcttgttcctcggccat

SEQ ID NO: 33 - DNA, 20 bp, Artificial Sequence
  Synthetic primer mSox11-F
tccaggtccttatccaccag

SEQ ID NO: 34 - DNA, 20 bp, Artificial Sequence
  Synthetic primer mSox11-R
gacgacctcatgttcgacct

SEQ ID NO: 35 - DNA, 20 bp, Artificial Sequence
  Synthetic primer mTrf-F
gctgtccctgacaaaacggt

SEQ ID NO: 36 - DNA, 21 bp, Artificial Sequence
  Synthetic primer mTrf-R
cggaaggacggtcttcatgtg

SEQ ID NO: 37 - DNA, 30 bp, Artificial Sequence
  Synthetic primer mTubb3-F
ccgaggccgagagcaacatgaatgacctgg

SEQ ID NO: 38 - DNA, 31 bp, Artificial Sequence
  Synthetic primer mTubb3-R
ccgattcctcgtcatcatcttcatacatctc

SEQ ID NO: 39 - DNA, 23 bp, Artificial Sequence
  Synthetic primer mUgt8-F
actccatatttcatgctcctgtg

SEQ ID NO: 40 - DNA, 21 bp, Artificial Sequence
  Synthetic primer mUgt8-R
aggccgatgctagtgtcttga

SEQ ID NO: 41 - DNA, 20 bp, Artificial Sequence
  Synthetic oligonucleotide shRNAp53
agtagattaccactggagtc

SEQ ID NO: 42 - DNA, 19 bp, Artificial Sequence
  Synthetic oligonucleotide shRNAp53
gactccagtggtaatctac

SEQ ID NO: 43 - DNA, 54 bp, Artificial Sequence
  Synthetic sequence from shp53 pLKO.1 puro vector from AddGene
ccgactccagtggtaatctacttcaagagagtagattaccactggagtcttttt

(19) United States
(12) Patent Application Publication
(10) Pub. No.: US 2015/0329913 A1
(43) Pub. Date: Nov. 19, 2015

(54) Identification of a 5-Gene Expression Signature Predicting Clinical Outcome of Patients with Brain Tumors

(71) Applicant: THE TRUSTEES OF COLUMBIA UNIVERSITY IN THE CITY OF NEW YORK, New York, NY (US)

(72) Inventors: Antonio Iavarone, New York, NY (US); Anna Lasorella, New York, NY (US)

(73) Assignee: THE TRUSTEES OF COLUMBIA UNIVERSITY IN THE CITY OF NEW YORK, NEW YORK, NY (US)

(21) Appl. No.: 14/652,787

(22) PCT Filed: Dec. 17, 2013
(86) PCT No.: PCT/US13/75884

§ 371 Date: Jun. 16, 2015

Related U.S. Application Data
(60) Provisional application No. 61/738,190, filed on Dec. 17, 2012.

(51) Int. Cl. C12Q 1/68; A61K 31/138; G01N 33/574; C12N 15/85; A01K 67/027

(52) U.S. Cl. CPC C12Q 1/6886; C12N 15/8509; A01K 67/0276; G01N 33/57407; A61K 31/138; C12Q 2600/118; C12Q 2600/158; A01K 2267/0331; A01K 2227/105; A01K 2217/058; G01N 2333/4703; G01N 2333/914; G01N 2333/4704

(57) ABSTRACT

High-grade gliomas are the most common brain tumors in humans and are essentially incurable. The defining hallmark of these high-grade gliomas is the presence within the tumor mass of highly tumorigenic cellular subpopulations that fuel tumor aggressiveness. Identification of the molecular mechanisms remains elusive. Therefore, there is a need for diagnostic markers to accurately determine the type of glioma and appropriate treatment. Gene delivery vehicles are provided that comprise an oncogene, IRES-Cre-ER cassette and a gene encoding an oligonucleotide that inhibits p53 including shp53. Methods are also provided for determining a diagnosis and for treatment of non-aggressive and aggressive gliomas.

### CROSS-REFERENCE TO RELATED APPLICATIONS

The present application claims the benefit of Provisional Application No. 61/738,190, filed Dec. 17, 2012 and is incorporated herein by reference.

### BACKGROUND

High-grade gliomas (HGG) are the most common brain tumors in humans and are essentially incurable (1). The defining hallmark of HGG is the presence within the tumor mass of highly tumorigenic cellular subpopulations, which fuel tumor aggressiveness. These cell subpopulations hijack several features of neural stem cells (NSCs) such as self-renewal and multi-lineage differentiation capacity and are commonly defined as glioma stem/initiating cells (GICs) (2, 3). GICs reside in a perivascular niche, a microenvironment that is critical to preserve the cancer stem cell state and tumor aggressiveness, and adhesion of GICs to endothelial cells is essential to support the stem cell-like characteristics of GICs (2, 4-7). Disruption of the intimate contacts established by GICs with tumor endothelial cells abrogates self-renewal and tumor-initiating capacity of GICs (4, 6, 8). It has been suggested that targeting the mechanisms driving the GIC state and maintaining the anchorage of these cells to the perivascular niche may provide important therapeutic value. However, the molecular identification of those mechanisms remains elusive.

It is important to accurately diagnose the type of glioma in a subject in order to plan and administer the most appropriate treatment program. Several studies have identified gene expression subgroups in human malignant glioma with the two most robust and alternative categories defined as proneural and mesenchymal and linked to better and worse clinical outcome, respectively (20, 26, 27). However there is still a need for further diagnostic markers to more accurately determine the type of glioma and the appropriate treatment.

### SUMMARY

The experiments described herein show that transduction of brain cells in malignant glioma in the hippocampus with a newly discovered lentivirus recapitulates mesenchymal tumors, and at the same time accomplishes the temporally controlled deletion of one or more floxed targeted genes, e.g., Id genes exclusively in the glioma tumor following a period of cancer initiation. It has been discovered that gliomas can be generated by transducing brain cells in the hippocampus in vivo with a gene delivery vehicle (e.g., a lentivirus vector such as HrasV12-Cre-ER-shp53 or pTomo-H-RasV12-IRES-CRe-ER-shp53) carrying a newly discovered lentivirus that (1) expresses oncogenic RAS (HrasV12) and (2) shRNA against the tumor suppressor Tp53 (shp 53), and (3) has been further modified to include a conditionally active Cre recombinase gene that enables the lentivirus to delete any floxed gene of interest when the Cre recombinase is activated by tamoxifen. This last property of the lentivirus allows assessing the therapeutic value of eliminating floxed gene from an established tumor. Therefore, methods for diagnosis and treatment of gliomas are provided. In certain embodiments, the gene delivery vehicle is a lentivirus vector comprising an oncogene (e.g., RAS, WNT, MYC, ERK, and TRK), IRES-Cre-ER cassette, and a gene encoding an oligonucleotide that inhibits p53 expression selected from the group comprising shp53, siRNA against p53, shRNA against p53, antisense RNA, and microRNA known as a HrasV12-Cre-ER-shp53 lentivirus vector.

Certain embodiments are directed to cells transduced with the gene delivery vehicle such as the HrasV12-Cre-ER-shp53 lentivirus vector. These cells that are transduced may be in the form of a glial cell. In other embodiments, the cells that are transduced comprise at least one floxed gene such as Id1, Id2, and Id3. Other embodiments are directed to a conditional ID-null mouse (Id1L/L; Id2L/L; Id3−/− mouse) and cells from it. Cells from the Id-null mouse can be transduced with the gene delivery vehicle of claim 1 comprising an oncogene, IRES-Cre-ER cassette, and a gene encoding an oligonucleotide that inhibits p53. Other animals may include an animal comprising one or more floxed genes, transduced with the gene delivery vehicle (e.g., a HrasV12-Cre-ER-shp53 lentivirus vector).

In other embodiments, methods are provided for (a) obtaining a transgenic animal comprising one or more floxed genes; (b) obtaining the gene delivery vehicle (e.g., a HrasV12-Cre-ER-shp53 lentivirus vector) comprising an oncogene, IRES-Cre-ER cassette, and a gene encoding an oligonucleotide that inhibits p53; (c) transducing cells in a target area of the animal with the gene delivery vehicle; (d) waiting a period of time sufficient for cancerous cells to form in the target area; (e) contacting the cancerous cells with tamoxifen in an amount sufficient to activate the IRES-Cre-ER cassette thereby deleting one more floxed genes; and (f) determining an effect of deleting the one more floxed alleles in the cancerous cells. The effect may be (i) a slowing of the growth rate of the cancerous cell, (ii) a slowing of the rate of metastasis, and (iii) lengthening survival.

In other embodiments, methods comprise (a) obtaining a sample of a glioma from a subject and (b) determining a level of expression of each protein selected from the group consisting of: TCF12/HEB, RAP1GAP, CDKN1C, ID2 and ID3 in the subject glioma sample, and (c) comparing the level of expression of each protein in the subject glioma sample to a known median level of expression of each of the corresponding proteins TCF12/HEB, RAP1GAP, CDKN1C, ID2 and ID3 in a standard glioma population, and (d) if the level of expression of TCF12/HEB, RAP1GAP and CDKN1C is significantly lower in the subject glioma sample compared to the known median for each corresponding protein in the standard glioma population, and if the level of each of ID2 and ID3 expression is significantly higher in the subject glioma sample than the standard glioma population, then it is possible to determine that subject glioma as an aggressive glioma carrying a very poor prognosis. Once a diagnosis is determined, it is then possible to treat the aggressive glioma in a subject in need thereof.

In other embodiments, if the level of expression of TCF12/HEB, RAP1GAP and CDKN1C is significantly higher in the subject glioma compared to the known median for each corresponding protein in the standard glioma population, then it is possible to diagnose the glioma as a non-aggressive glioma carrying a better prognosis. In each of these embodiments, the level of expression of each protein is determined by measuring the level of mRNA encoding each respective protein in the subject glioma sample.

The level of expression of each of the proteins TCF12/HEB, RAP1GA, CDKN1C, ID2, and ID3 in the glioma can be determined by either determining the level of each of the proteins, or the level of cNDA for each respective protein, or the level of mRNA encoding each respective protein in the glioma.

Other methods are provided in certain embodiments where a sample of a glioma is obtained from a subject. The level of expression of each of the proteins TCF12/HEB, RAP1GAP, CDKN1C, ID2, and ID3 is then determined in the glioma sample. If expression of TC12/HEB, RAP1GAP, and CDKN1C cannot be detected in the glioma sample, and if expression of ID2 and ID3 is detectable in the glioma sample, then the glioma is diagnosed as an aggressive glioma, and treatment of the aggressive glioma in the subject may follow.

On the other hand, methods are provided where a sample of a glioma is obtained from a subject. The level of expression of each of the proteins TCF12/HEB, RAP1GAP, CDKN1C, ID2, and ID3 is then determined in the glioma sample. If expression of TC12/HEB, RAP1GAP, and CDKN1C can be detected in the glioma sample, and if expression of ID2 and ID3 is undetectable in the glioma sample, then the glioma is diagnosed as a non-aggressive glioma, and treatment of the non-aggressive glioma in the subject may follow. In these embodiments, the level of expression is determined using immunohistochemistry or PCR.

### BRIEF DESCRIPTION OF THE DRAWINGS

The present invention is illustrated by way of example, and not by way of limitation, in the figures.

FIG. 1. HGG arising in mice injected with Ras-V12-IRES-Cre-ER-shp53 lentivirus. (A) Hematoxylin and Eosin staining and immunophenotype of a representative tumor lesion in the hippocampus of Id-cTKO mice 12 days after stereotaxic injection with Ras-V12-IRES-Cre-ER-shp53 lentivirus. Adjacent sections were immunostained using the indicated antibodies. Scale bars 500 μm. (B) Representative microphotographs of Hematoxylin and Eosin staining of advanced Ras-V12-IRES-Cre-ER-shp53 generated tumors showing histological features of high-grade glioma. The arrow indicates a multinucleated glioblastoma giant cell. Arrowheads point at clusters of tumor cells infiltrating the normal brain. N, necrotic foci. Scale bars: 500 μm (upper left panel); 100 μm (upper right and lower panels). (C) Immunofluorescence staining on representative brain sections from mice injected with Ras-V12-IRES-Cre-ER-shp53 lentivirus sacrificed after the manifestation of neurological symptoms. Glioma and stem cell markers (Nestin, Olig2 and GFAP), ID1, ID2, the proliferation marker Ki67 and vascular endothelial cell marker CD31 are widely expressed. βIII-tubulin is present in scattered cells. The arrow indicates the soma of a neuron. Scale bars: 100 μm (Nestin, Olig2, βIII-tubutin, GFAP, Ki67); 200 μm (ID1, ID2, and CD31). (D) Double immunofluorescence staining for ID1 (green) and ID2 (red) shows co-expression of ID1 and ID2 in the vast majority of tumors cells. Nuclei were counterstained with DAPI (blue). Arrowheads and arrows point at double positive and single positive cells, respectively. Scale bar: 20 μm.

FIG. 2. Ablation of Id in cancer cells affects tumor maintenance in Ras-V12-IRES-Cre-ER-shp53 HGG. (A) Kaplan-Meier survival curves of Id-cTKO mice injected intracranially with Ras-V12-IRES-Cre-ER-shp53 lentivirus that had received systemic tamoxifen or oil. Points on the curves indicate deaths; n=13 for oil and n=11 for tamoxifen; p=0.002. (B) Hematoxylin and Eosin staining and immunophenotype on adjacent sections of representative brains from Id-cTKO mice treated with tamoxifen or oil and sacrificed 6 weeks after stereotaxic injection with Ras-V12-IRES-Cre-ER-shp53 lentivirus. T, tumor. Scale bars: 500 μm (H&E, ID1, Ki67, Nestin); 20 μm (SSEA1). (C) Quantification of tumor volume in mice as in (B). n=3 per each treatment group; p=0.0381. (D) Quantification of Ki67⁺ cells in tumors as in (B). n=3 per each treatment group; p=0.0024. (E). Quantification of SSEA1⁺ cells in tumors as in (B). n=3 per each treatment group; p=0.0002. Data are Mean±SD.

FIG. 3. The effect of Id ablation in Ras-V12-IRES-Cre-ER-shp53 GICs in vitro and after orthotopic transplantation in vivo. (A) Cells isolated from Ras-V12-IRES-Cre-ER-shp53 gliomas generated in Id-cTKO mice were cultured in medium containing EGF and FGF-2 for 3 passages and immunostained using antibodies against Nestin, SSEA1 and ITGα6. Scale bars: 20 mm. (B) Microphotographs of tumor spheres from GICs 14 days after plating at <1 cell/μl in semi-adherent conditions. GICs isolated from advanced Ras-V12-IRES-Cre-ER-shp53 gliomas in Id-cTKO or wild type control mice were cultured in medium containing EGF and FGF-2 for 2 passages and then subjected to treatment with vehicle or 4-OHT for 5 days. Dissociated cells were assayed by microscopic analysis for sphere formation. Scale bars: 500 mm (upper panels); Scale bars: 250 mm (lower panels); (C) Quantification of gliomaspheres as a percentage of plated cells from cultures with the indicated genotypes and treatments. Data represent the Mean±SD of triplicate samples; p=0.003. (D) Regression plot for cells isolated from controls or tamoxifen treated tumors. Cells from two independent tumors/per treatment were plated at limiting dilution (1-400/well) in triplicate plates; Data represent the Mean±SD; p=0.006. Experiments were repeated twice. (E) Hematoxylin and Eosin staining of representative brain sections of immunodeficient mice subjected to stereotaxic injection with Ras-V12-IRES-Cre-ER-shp53 Id-cTKO GISs and treated with oil or tamoxifen. Scale bars: 250 mm.

FIG. 4. Ablation of Id disrupts adhesion of GICs to endothelial cells in the perivascular niche. (A) Immunostaining for SSEA1 (red) and CD31 (green) on a representative Ras-V12-IRES-Cre-ER-shp53 HGG in Id-cTKO mice 6 weeks after treatment with tamoxifen or oil. Nuclei were counterstained with DAPI (blue). (B) Quantification from (A) of the percentage of SSEA1⁺ cells within 10 μm from CD31⁺ cells. n=4 per each treatment group; p=0.0005. (C) Immunostaining for SSEA1 (red) and CD31 (green) on representative Ras-V12-IRES-Cre-ER-shp53 HGG tumors collected from Id-cTKO mice 7 days after treatment with tamoxifen or oil. Nuclei were counterstained with DAPI (blue). (D) Quantification from (C) of the percentage of SSEA1⁺ cells within 10 μm from CD31⁺ cells. n=4 per each treatment group; p=0.0005. (E) Immunostaining for SSEA1 (red) and Cleaved Caspase-3 (green) on representative Ras-V12-IRES-Cre-ER-shp53 HGG from Id-cTKO mice 7 days after treatment with tamoxifen or oil. Nuclei were counterstained with DAPI (blue). Data represent the Mean±SD. Asterisks indicate the lumen of blood vessels; arrows indicate SSEA1⁺ cells within 10 μm from CD31⁺ cells; arrowhead indicate SSEA1⁺ cells >10 μm from CD31⁺ cells. Scale bars: 20 μm.

FIG. 5. Expression of mesenchymal markers and Rap1GAP mRNA in HGG arising in mice injected with Ras-V12-IRES-Cre-ER-shp53 lentivirus. (A) Immunofluorescence staining for Fibronectin and CTGF (red) of representative glioma sections from mice treated with oil or tamoxifen and sacrificed 6 weeks after lentiviral transduction of the dentate gyrus. Nuclei were counterstained with DAPI (blue). Scale bars: 50 μm. (B) Normalized values of Rap1GAP mRNA from microarray gene expression profiles of glioma from mice treated with oil or tamoxifen and sacrificed 6 weeks after lentiviral transduction. Data represent the Mean±SD; n=4 mice for each condition.

FIG. 6. Active RAP1 rescues the loss of GSC adhesion caused by Id ablation. (A) Immunostaining for Rap1GAP (red) of representative tumor lesions in Id-cTKO mice treated with tamoxifen or oil and sacrificed 6 weeks after stereotaxic injection of Ras-V12-IRES-Cre-ER-shp53 lentivirus. Nuclei were counterstained with DAPI (blue). T, tumor. Scale Bars: 100 μm. (B) Two hundred glioma spheres from Id1L/L; Id2L/L; Id3L/L iGICs co-infected with lentiviral vectors expressing EGFRvIII-IRES-Cre-ER-shp53 and pLOC-RAP1A-G12V; Q63E-GFP or pLOC-GFP were treated with vehicle or 4-OHT for 4 days and assayed for adhesion to brain derived endothelial cells for 24 h. Scale Bars: 100 μm. (C) Quantification of the percentage of glioma spheres from (B) adhering to a monolayer of brain endothelial cells after an adhesion period of 24 h as determined by fluorescent microscopy review. Data represent the Mean±SD of quadruplicate samples. p=0.0114. (D) Id1L/L; Id2L/L; Id3L/L iGSCs co-infected with lentiviral vectors expressing EGFRvIII-IRES-Cre-ER-shp53 and pLOC-RAP1A-G12V; Q63E-GFP or pLOC-GFP were treated with vehicle or 4-OHT for 4 days. Gliomaspheres were dissociated and single cells plated at density of <1 cell/μl in 96 well plates in triplicates. The number of gliomaspheres was scored 10 days later. Data represent the Mean±SD. p=9.6489E-05.

FIG. 7. Expression of Rap1GAP impacts human GICs properties. (A) Analysis of RAP1 activity in human GICs transduced with pLOC-Rap1GAP-GFP or pLOC-GFP lentivirus. Proteins from pull down assay were analyzed by immunoblotting using RAP1 antibody (RAP1-GTP). Total cell lysates were analyzed by immunoblotting using the indicated antibodies. α-tubulin is shown as loading control. Asterisk indicates a non-specific band. (B) Microphotographs of human GICs treated as in (A) and assayed for adhesion to endothelial cells. Scale Bars: 200 μm. (C) The number of adherent GFP⁺ cell was determined by high-power microscopy review. Data represent the Mean±SD of quadruplicate samples from two independent experiments. p=0.001. (D) Regression plot for GICs transduced with pLOC-Rap1GAP-GFP or pLOC-GFP lentivirus. Cells from two independent infections were plated at limiting dilution (1-500/well). Each data point represents the Mean±SD; n=3 96 well plates for each condition; p=0.003. Experiments were repeated twice. (E) The size of tumor spheres obtained from cells treated as in (A) was determined by microscopy review. Data represent the Mean±SD. n=60 spheres from triplicates for each condition; p=0.0012. (F) Microphotographs of representative tumor spheres from vector and Rap1GAP transduced GICs. Scale bars: 141 μm for Vector and 40.2 μm for Rap1GAP.

FIG. 8. Loss of cell adhesion by Rap1GAP in GICs is independent of proliferation. (A) Western blot analysis for Rap1GAP and p27^{Kip1} expression in GICs transduced with pLOC-Rap1GAP-GFP, pLOC-p27^{KIP1}-GFP or pLOC-GFP. β-Actin is shown as loading control. (B) Microphotographs of human GSCs transduced with pLOC-Rap1GAP-GFP, pLOC-p27^{KIP1}-GFP or pLOC-GFP and assayed for adhesion to endothelial cells. Scale bars: 100 μm. (C) The number of adherent GFP⁺ cells was determined by high-power microscopy review. Data represent the Mean±SD of triplicate samples from two independent experiments. p=2.4069E-05. (D) The number of tumor spheres obtained from human GICs treated as in (G) and seeded at <1 cell/μl was determined by microscopy review. Data represent the Mean±SD of triplicate samples. p=5.05863E-05.

FIG. 9. The ID-RAP1 pathway carries prognostic value in human malignant glioma. (A) The expression of RAP1 GAP is significantly down regulated in 81 samples from human glioblastoma (class 2, dark blue) compared with 23 samples from non-tumor human brain (class 1, light blue), p 3.83E-22. (B) Kaplan-Meier analysis comparing survival of patients carrying HGG expressing high TCF12/HEB, RAP1GAP, CDKN1C and low ID2 and ID3 (green line) or low TCF12IHEB, RAP1GAP, CDKN1C and high ID2 and ID3 (blue line) and the overall patient population (red line). p<0.001.

FIG. 10. The ID-Rap1GAP-RAP1 pathway controls residency of GICs in the perivascular niche of malignant glioma. High levels of ID proteins inhibit transcriptional activation of the Rap1GAP gene by bHLH transcription factors in GICs. The balance of the RAP1-GTPase is shifted towards active RAP1-GTP that activates integrin signaling and anchorage of glioma cells to the tumor endothelium. Loss of ID activity de-represses bHLH-mediated activation of Rap1GAP expression with inhibition of RAP1-GTPase and relative accumulation of RAP1 as RAP1-GDP. Loss of RAP1-GTP results in the release of GICs from the perivascular space with ensuing loss of stem and tumor initiating capacity.

FIG. 11. Intracranial transduction of a modified pTomo lentiviral vector in the mouse hippocampus targets GFAP-positive but not NeuN-positive cells. (A) Stereotaxic injection of pTomo-GFP lentivirus was done to target the dentate gyrus of the mouse hippocampus and brain sections were prepared five days later. (left), Double immunofluorescence for GFP (green) and GFAP (red). (right), Double immunofluorescence for GFP (green) and NeuN (red). (B) Schematic representation of pTomo-H-RasV12-IRES-Cre-ER-shp53 lentiviral vector. Activation of Cre recombinase by tamoxifen results in deletion of the floxed Id genes exclusively in tumor cells. (C) High magnification microphotographs for Hematoxylin and Eosin staining and immunophenotype of the representative tumor lesion in FIG. 1A. Id-cTKO mice were sacrificed 12 days after stereotaxic injection with Ras-V12-IRES-Cre-ER-shp53 lentivirus. Adjacent sections were immunostained using the indicated antibodies. Scale bars: 50 μm.

FIG. 12, Treatment of Id-cTKO glioma-bearing mice with tamoxifen results in loss of expression of ID proteins and Ki67 but not H-RasV12 in glioma cells. Brain tumors from mice treated with oil or tamoxifen for four days were stained for ID2 (A), ID1 (B) and Ki67 (C). B, brain; T, tumor. (D) Immunostaining for H-RasV12 (red) and Id proteins (green) in mice treated with vehicle or tamoxifen and sacrificed 6 weeks after lentiviral transduction. Scale bars: 100 μm (A, B, C); 20 μm (D).

FIG. 13. The effect of ablation of Id genes in malignant glioma induced by Ras-V12-IRES-Cre-ER-shp53. (A) Expansion of glioma cells retaining ID1 and ID2 after re-growth of tamoxifen-treated Ras-V12-IRES-Cre-ER-shp53 glioma. Immunostaining for ID1 and ID2 (red) of a representative tumor lesion in mice treated with vehicle or tamoxifen and sacrificed after the manifestation of neurological symptoms. Nuclei were counterstained with DAPI. (B) Double immunostaining for ID protein (red) and the stem cell marker Nestin (green) in mice treated with vehicle or tamoxifen and sacrificed 6 weeks after lentiviral transduction. (C) Tumor explants from advanced Ras-V12-IRES-Cre-ER-shp53 glioma generated in Id-cTKO mice were cultured in medium containing EGF and FGF-2 in the presence or the absence of 4-OHT for 5 days. Fixed tissues were immunostained with anti-Nestin antibody and analyzed by confocal microscopy. (D) Double immunostaining for ID protein (red) and the astrocytic marker GFAP (green) in mice treated with vehicle or tamoxifen and sacrificed 6 weeks after lentiviral transduction. Scale bars: 100 μm (A); 25 μm (insets A); 50 m (B, C, D).

FIG. 14. The expression of ID proteins in GCSs. (A) Immunostaining for SSEA1 (red) and ID1 (green) on a representative Ras-V12-IRES-Cre-ER-shp53 HGG in Id-cTKO mice. Nuclei were counterstained with DAPI (blue). (B) Immunostaining for SSEA1 (red) and ID2 (green) on representative Ras-V12-IRES-Cre-ER-shp53 HGG in Id-cTKO mice. Nuclei were counterstained with DAPI (blue). (C) Quantification of the fraction of SSEA1 positive cells that express ID1 or ID2 proteins. Scale bars: 20 μm.

FIG. 15. Ablation of Id genes disrupts adhesion of GSCs to endothelial cells in the perivascular niche. (A) Immunostaining for ITGα6 (red) and CD31 (green) on representative Ras-V12-IRES-Cre-ER-shp53 HGG in Id-cTKO mice treated with tamoxifen or oil. Nuclei were counterstained with DAPI (blue). Asterisks indicate the lumen of blood vessels; arrows indicate ITGα6⁺ cells within 10 μm from CD31⁺ cells; arrowhead indicate ITGα6⁺ cells >10 μm from CD31⁺ cells. (B) Quantification of the percentage of ITGα6^{°} cells within 10 μm from CD31⁺ cells. n=4 per each treatment group; p=7.966E-07. (C) Immunostaining for cleaved caspase-3 on representative Ras-V12-IRES-Cre-ER-shp53 HGG in Id-cTKO mice treated with tamoxifen or oil. Nuclei were counterstained with hematoxylin. (D) Quantification of the number of cleaved caspase-3⁺ cells/field using a 40× objective. n=3 per each treatment group. Scale bars: 20 μm (A); 200 Sm (C).

FIG. 16. Ablation of Id genes does not affect expression of IL-6. (A) Immunostaining for IL-6 (red) on representative Ras-V12-IRES-Cre-ER-shp53 HGG in Id-cTKO mice treated with tamoxifen or oil. Nuclei were counterstained with DAPI. (D) Quantification of the fluorescence intensity for IL-6 in Ras-V12-IRES-Cre-ER-shp53 HGG in Id-cTKO mice treated with tamoxifen or oil. Bars indicate Mean±SD. n=3 for each group. Scale bars: 50 m.

FIG. 17. Constitutive EGFR signaling requires Id activity to maintain the transformed phenotype. (A) Microphotographs of Id1L/L; Id2L/L; Id3L/L astrocytes transformed by the expression of EGFRvIII-Cre-ER-shp53 lentivirus (iGSCs) treated with vehicle or 4-OHT. Arrow indicates the soma of a neuron; arrowheads indicate oligodendrocytes. (B) Western blot analysis of cells treated as in (A) shows efficient recombination of Id1, Id2 and Id3 and induction of Rap1GAP by 4-OHT. (C) Cells treated as in (A) were assayed by qRT-PCR for Id1, Id2 and Id3, Rap1gap and the NSC marker nestin along with neural lineage differentiation markers. Data represent the Mean±SD of triplicate amplification reactions. (D) Analysis of cleaved caspase-3 in iGSCs treated with vehicle or 4-OHT. Total cell lysates were analyzed by SDS-PAGE and immunoblotting using the indicated antibodies. Cells treated with Etoposide were used as positive control for cleaved caspase-3. α-tubulin is shown as loading control. (E) Analysis of RAP1 activity in Id1L/L; Id2L/L; Id3L/L iGSCs transduced with a pLOC-GFP-RAP1AG12V; Q63E or pLOC-vector-GFP lentivirus using the RAP1 pull down assay. Protein samples were analyzed by SDS-PAGE and immunoblotting using RAP1 antibody (RAP1-GTP). Total cell lysates were analyzed by SDS-PAGE and immunoblotting using the indicated antibodies. α-tubulin is shown as loading control. (F) Bright field microphotographs of the endothelial cell-EGFRvIII-Cre-ER-shp53 iGSC co-cultures presented in the adhesion assay in FIG. 6B. Scale bars: 200 μm (A, upper panels); 50 μm (A, lower panels); 100 μm (F).

FIG. 18. Expression of Rap1GAP in human GICs does not affect cell proliferation (A) Immunostaining for BrdU in human GICs infected with pLOC-Rap1GAP-GFP, pLOC-p27^{KIP1}-GFP or pLOC-GFP lentiviruses. (B) The number of BrdU⁺ cells was determined in triplicate samples. At least 1000 cells were scored. p=9.51609E-05. (C) Analysis of cleaved caspase-3 in human GICs transduced with pLOC-Rap1GAP-GFP or pLOC-GFP lentivirus. Total cell lysates were analyzed by SDS-PAGE and immunoblotting using the indicated antibodies. Cells treated with Staurosporine were used as positive control for cleaved caspase-3. α-tubulin is shown as loading control. Scale bars: 200 μm.

FIG. 19. The expression of RAP1GAP is reduced in human glioma. (A) The expression of RAP1GAP is significantly down regulated in 19 samples from human anaplastic astrocytoma (class 2, dark blue) compared with 23 samples from non-tumor human brain (class 1, light blue), p=7.21E-9. (B) The expression of RAP/GAP is significantly down regulated in 45 samples from human astrocytoma (class 2, dark blue) compared with 6 samples from non-tumor human brain (temporal lobe, class 1, light blue), p=5.51E5. (C) The expression of RAP1GAP is significantly down regulated in 50 samples from human oligodendroglioma (class 2, dark blue) compared with 23 samples from non-tumor human brain (class 1, light blue), p=1.67E-16.

FIG. 20. High ID1 expression correlates with better survival of patients within the proneural subclass of HGG. Kaplan-Meier analysis comparing survival of patients expressing high (red line) or low (blue line) levels of ID1 in proneural (A), mesenchymal (B) or the overall (C) population of patients with HGG.

Before the present embodiments of the invention are described, it is to be understood that the inventions are not limited to the particular processes, compositions, or methodologies described, as these may vary. The terminology used in the description is for the purpose of describing the particular versions or embodiments only, and is not intended to limit the scope of the present invention. Unless otherwise defined, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

### DETAILED DESCRIPTION

In the following description, for the purposes of explanation, numerous specific details are set forth in order to provide a thorough understanding of the present invention. It will be apparent, however, to one skilled in the art that the present invention may be practiced without these specific details.

It has now been discovered that the transduction of brain cells in an animal genetically engineered to express one or more floxed target genes with a newly generated lentivirus (HrasV12-Cre-ER-shp53 or pTomo-H-RasV12-IRES-Cre-ER-shp3) in vivo recapitulates mesenchymal tumors, the most aggressive subtype of HGG, and simultaneously accomplishes the conditional (i.e., tamoxifen-activated), temporally controlled deletion of the floxed targeted genes exclusively in the glioma tumor following a period of cancer initiation. It has been further discovered that inhibiting the expression of Id1, Id2 and Id3 genes in cancer cells, such as glioma, reduces the aggressiveness of the cancer.

To study the role of the Id genes in cancer, a novel conditional Id-null mouse (Id1L/L; Id2L/L; Id3−/−) having floxed Id1 and Id2 genes and wherein Id3 has been knocked out, was made. The Id-null mice were transduced in vivo with a newly discovered lentivirus vector comprising an IRES-Cre-ER cassette (also herein referred to as the Cre-recombinase fused to the Estrogen Receptor transactivation domain (ER) feature or Cre-recombinase) linked to cDNA encoding the oncogene HrasV12 and further comprising shRNA against the tumor suppressor Tp53 (shp53). Although the HrasV12 oncogene was used in the examples, any oncogene or combination of oncogenes can be inserted into the vector. Further, although shRNA was used in the examples, any inhibitory oligonucleotide that blocks p53 expression can be used. After a period of time, an aggressive mesenchymal tumor developed at the site where the vector was injected. Mesenchymal tumors are the most aggressive subtype of high grade glioma, HGG. Upon exposure to tamoxifen that activates the Cre-recombinase enzyme, the floxed Id1 and Id2 genes were deleted. Blocking expression of the Id genes caused the mesenchymal tumors to become less aggressive. This same vector can be used to cause tumors in targeted cells in other areas of the body, as it is not specific for any particular type of cell. Moreover, any gene of interest can be floxed and thereby selectively deleted upon tamoxifen exposure in the presently described animal model.

In order that the invention may be readily understood and put into practical effect, particular preferred embodiments will now be described by way of the following non-limiting examples.

### 1. Definitions

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwart, Thomas M. Jessell editors. McGraw-Hill/Appleton & Lange: New York, N. (2000).

The term “conditional Id-null mouse” as used herein means a transgenic mouse that contains additional, artificially-introduced genetic material in every cell. The conditional Id-null transgenic mouse described herein is a genetic model for Id ablation In which the null mice carry floxed Id1 and Id2 alleles and a constitutive Id3-knockout allele (Id1L/L; Id2L/L; Id3−/−). Glioma cells of this conditional Id null mouse are then infected with the lentiviral vectors carrying a CRE recombinase gene. In the Cre-ER system the Cre recombinase enzyme becomes active and permanently deletes the floxed Id1 and Id2 genes when stimulated by tamoxifen.

The term “floxed” as used herein, describes the sandwiching of a DNA sequence between two lox P sites, and is a contraction of the phrase “flanked by LoxP”. Recombination between LoxP sites is catalyzed by Cre recombinase. Floxing a gene allows it to be deleted (knocked out), translocated, or inverted in a process called Cre-Lox recombination. In other words, the enzyme Cre recombinase deletes genes. It cuts DNA fragments (genes) flanked by LoxP sites. Further, these knockouts can be inducible in the Cre-ER. In several mouse studies, tamoxifen is used to induce the Cre recombinase. Tamoxifen binds to ER and disrupts its interactions with the chaperones. That allows the Cre-ER fusion protein to enter the nucleus and act on the floxed gene.

A “gene delivery vehicle” as used herein refers to a construct which is capable of delivering, and, within some embodiments expressing, one or more gene(s) or nucleotide sequence(s) of interest in a host cell. Representative examples of such vehicles include viral vectors such as retroviral vectors which include lentiviruses.

A “glioma” as used herein is a type of tumor that starts in the brain or spine. It is called a glioma because it arises from nonmalignant glial precursor cells. The most common site of gliomas is in the brain. Gliomas make up ˜30% of all brain and central nervous system tumors and 80% of all malignant brain tumors.

An “IRES (internal ribosome entry site) sequence”, as used herein, may be used to produce more than one polypeptide from a single gene transcript. An IRES (or other suitable sequence) is used to produce a protein that contains more than one polypeptide chain or to express two different proteins from or within the same cell.

“Lentivirus” as used herein, is a genus of viruses of the Retroviridae family, characterized by a long incubation period. Lentiviruses can deliver a significant amount of viral RNA into the DNA of the host cell and have the unique ability among retroviruses of being able to infect non-dividing cells, so they are one of the most efficient methods of a gene delivery vector. HIV, SIV, FIV, EIAV, and Visna are all examples of lentiviruses.

“Oligonucleotides” as used herein, are short, single-stranded DNA or RNA molecules that have a wide range of applications in genetic testing, research, and forensics. Commonly made in the laboratory by solid-phase chemical synthesis, these small bits of nucleic acids can be manufactured with any user-specified sequence, and so are vital for artificial gene synthesis, polymerase chain reaction (PCR), DNA sequencing, library construction and as molecular probes. In nature, oligonucleotides are usually found as small RNA molecules that function in the regulation of gene expression (e.g. microRNA), or are degradation intermediates derived from the breakdown of larger nucleic acid molecules.

An “oncogene” as used herein, is a gene that has the potential to cause cancer or induce cancer progression. In tumor cells, they are often mutated or expressed at high levels. Most normal cells undergo a programmed form of death (apoptosis). Activated oncogenes can cause those cells designated for apoptosis to survive and proliferate instead. Any oncogene known in the art, or a combination of oncogenes, can be used in embodiments of the present invention, including but not limited to RAS, WNT, MYC, ERK, and TRK and are described herein.

A “plasmid” as used herein, is a small DNA molecule that is physically separate from, and can replicate independently of, chromosomal DNA within a cell. Most commonly found as small circular, double-stranded DNA molecules in bacteria, plasmids are sometimes present in archaea and eukaryotic organisms. In nature, plasmids carry genes that may benefit survival of the organism (e.g. antibiotic resistance), and can frequently be transmitted from one bacterium to another (even of another species) via horizontal gene transfer. Artificial plasmids are widely used as vectors in molecular cloning, serving to drive the replication of recombinant DNA sequences within host organisms. Plasmid sizes vary from 1 to over 1,000 kbp. The number of identical plasmids in a single cell can range anywhere from one to thousands under some circumstances. Plasmids can be considered part of the mobilome because they are often associated with conjugation, a mechanism of horizontal gene transfer.

A “provirus” as used herein, is a virus genome that is integrated into the DNA of a host cell. This state can be a stage of virus replication, or a state that persists over longer periods of time as either inactive viral infections or an endogenous retrovirus. In inactive viral infections the virus will not replicate itself but through replication of its host cell. This state can last over many host cell generations. Endogenous retroviruses are always in the state of a provirus. When a (nonendogenous) retrovirus invades a cell, the RNA of the retrovirus is reverse-transcribed into DNA by reverse transcriptase, then inserted into the host genome by an integrase. Proviruses may account for approximately 8% of the human genome in the form of inherited endogenous retroviruses. A provirus not only refers to a retrovirus but is also used to describe other viruses that can integrate into the host chromosomes, another example being adeno-associated virus. Not only eukaryotic viruses integrate into the genomes of their hosts; many bacterial and archaeal viruses also employ this strategy of propagation. All families of bacterial viruses with circular (single-stranded or double-stranded) DNA genomes or replicating their genomes through a circular intermediate (e.g., tailed dsDNA viruses) have temperate members. In the case of bacterial viruses (bacteriophages), proviruses are often referred to as prophages.

The term “retrovirus” as used herein, is well known in the art, and includes single-stranded, positive sense, enveloped RNA viruses that include, e.g., the genus Gammaretrovirus (e.g., murine mammary tumor virus); the genus Epsilonretrovirus; the genus Alpharetrovirus (e.g., avian leukosis virus); the genus Betaretrovirus; the genus Deltaretrovirus (e.g., bovine leukemia virus; human T-lymphotrophic virus (HTLV)); the genus Lentivirus; and the genus Spumavirus.

The term “shRNA” as used herein, is well known in the art, and refers to a small hairpin RNA or short hairpin RNA (shRNA) that is a sequence of RNA that makes a tight hairpin turn that can be used to silence target gene expression via RNA interference (RNAi). Expression of shRNA in cells is typically accomplished by delivery of plasmids or through viral or bacterial vectors. Short hairpin RNA (shRNA): The shRNA hairpin structure is cleaved by the cellular machinery into siRNA. Small hairpin RNA (shRNA) is synonymous with short hairpin RNA. DNA encoding a shRNA is can be included on a plasmid and operably linked to a promoter. This plasmid can be introduced into cells in which inhibition of expression a target sequence is desired. This plasmid is usually passed on to daughter cells, enabling inheritance of the gene silencing. Once produced or present in a cell, the hairpin structure of shRNA is cleaved by cellular machinery into siRNA. In certain embodiments, glioma were induced in brain cells in the hippocampus of Id-null mice after transduction with a viral vector comprising cDNA encoding the oncogene HrasV12 and a shRNA against the tumor suppressor Tp53 (hereafter shp53).

The term “significantly higher” as used herein, means that the levels of expression of a protein (herein a protein in the group TCF12/HEB, RAP1GAP, CDKN1C, ID2 and ID3) in a sample of cancer, such as a glioma, from a subject is at least 50% higher than the median levels of expression of the same protein(s) in a standard cancer (glioma) population. Such a standard glioma population contains a statistically significant number of glioma samples from the same species. In the examples herein, the standard glioma population had about 1000 subjects. On the other hand, the term “significantly lower” as used herein, means that levels of expression of a protein (TCF12/HEB, RAP1GAP, CDKN1C, ID2 and ID3) in the cancer (glioma) from a subject is at least 50% lower than the median level of expression of the same protein (s) in the standard cancer (glioma) population. The values are always related to the median of expression in a large number of gliomas used as a reference.

The term “standard glioma population” as used herein, refers to a population of gliomas that is large enough to be statistically significant for median levels of protein expression or other parameter being assayed. In the examples here, the standard glioma population used for the expression profiles is a population of 1,043 newly diagnosed HGG patients from the datasets. For embodiments of the diagnostic methods, a standard glioma population can be different from the 1,043 population used herein, as long as it is a large enough population to be statistically relevant for the measurements/conclusions being made.

The terms “subject,” “host,” and “patient,” as used herein, are used interchangeably and mean an animal being treated with the present compositions, including, but not limited to, simians, humans, avians, felines, canines, equines, rodents, bovines, porcines, ovines, caprines, mammalian farm animals, mammalian sport animals, and mammalian pets.

The term, “tamoxifen” as used herein, is an antagonist of the estrogen receptor in breast tissue via its active metabolite, hydroxytamoxifen. In other tissues such as the endometrium, it behaves as an agonist, and thus may be characterized as a mixed agonist/antagonist.

The term “treating” as used herein means slowing, stopping or reversing the progression of a disease, particularly a glioma (e.g., non-aggressive or aggressive). As used herein, the terms “treatment,” “treating,” and the like, as used herein refer to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or adverse effect attributable to the condition or disease. “Treatment,” includes any treatment of a condition or disease in a mammal, particularly in a human, and includes: (a) preventing the condition or disease or symptom thereof from occurring in a subject which may be predisposed to the condition or disease but has not yet been diagnosed as having it; (b) inhibiting the condition or disease or symptom thereof, such as, arresting its development; and (c) relieving, alleviating, mitigating or ameliorating the condition or disease or symptom thereof, such as, for example, causing regression of the condition or disease or symptom thereof.

The term “transduction” as used herein means the process by which foreign DNA is introduced into a target cell via a gene delivery vehicle such as a viral construct, preferably a lentivirus vector in certain embodiments. Transduction does not require cell-to-cell contact (which occurs in conjugation), and it is DNAase resistant (transformation is susceptible to DNAase). Transduction is a common tool used by molecular biologists to stably introduce a foreign gene into the genome of a targeted cell in the host. In this case, gene delivery vehicle (i.e., HrasV12-Cre-ER-shp53) is introduced into the brain cells of the hippocampus.

As used herein, “therapeutically effective amount” means an amount sufficient to treat a subject afflicted with a tumor (e.g., a glioma) or to alleviate a symptom or a complication associated with the tumor.

“Viral vectors” as used herein are a tool commonly used by molecular biologists to deliver genetic material into cells. This process can be performed inside a living organism (in vivo) or in cell culture (in vitro). Viruses have evolved specialized molecular mechanisms to efficiently transport their genomes inside the cells they infect. Delivery of genes by a virus is termed transduction and the infected cells are described as transduced. Viral vectors include not only lentiviruses but any viral vectors as described herein.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### 2. Overview

It has been discovered that inhibiting the expression of Id1, Id2, and Id3 genes in cancerous tumors, such as glioma, reduces the aggressiveness of the gliomas. To study the role of the Id genes in glioma, a novel Id-null mouse was made by crossing mice harboring floxed alleles of Id1 (Id1L/L) and Id2 (Id2L/L) with constitutive Id3 knockout mice (Id3−/−) to generate Id1L/L; Id2L/L; Id3−− (Id-cTKO) mice (13). Certain embodiments are directed to the new conditional Id1L/L; Id2L/L; Id3−/− mice. Glioma was induced in brain cells in the hippocampus of Id-null mice after transduction with a viral vector comprising cDNA encoding the oncogene HrasV12 and shRNA against the tumor suppressor Tp53 (hereafter shp53), which vector further comprised a conditionally active Cre recombinase to inactivate the floxed Id1 and Id2 genes in the presence of tamoxifen. The new lentivirus vector used in the experiments is referred to herein as “HrasV12-Cre-ER-shp53,” or “pTomo-H-RasV12-IRES-Cre-ER-shp53” lentiviral vectors. After a period of time allowing the glioma to develop, tamoxifen was administered to initiate the blocking of expression of the two floxed Id genes. The results showed that blocking expression of Id1 and Id2 (but the Cre-recombinase) and knocking out Id3, changed the HGG to a nonaggressive form of glioma. Certain embodiments are directed to the Id-null mouse; the new viral vectors herein described comprising an oncogene, a cre-recombinase and an oligonucleotide that inhibits floxed target gene expression; and Id null mice and cells from them that have been transduced with a vector of the present invention.

The results described herein show that (1) deletion of three Id genes induced rapid release of glioma-initiating cells (GICs) from the perivascular niche followed by tumor regression, and (2) that the displacement of GICs from the perivascular niche is implemented by derepression of Rap1GAP (as a result of Id gene deletion), with consequent inhibition of the activity of RAP1, a master regulator of cell adhesion. These effects lead to depletion of GICs and decline of glioma-initiating capacity.

TCF12/HEB, RAP1GAP, CDKN1C are key components of the ID-bHLH transcriptional network in neural stem cells. It has now been discovered that there is a “five-gene signature set” (TCF12/HEB, ID2, ID3, CDKN1C, RAP1GAP) the relative expression of which segregates two sub-groups of glioma patients with markedly divergent clinical outcomes. Gliomas that have significantly elevated expression of both ID2 and ID3, and significantly reduced expression of the bHLH transcription factor TCF12/HEB and its targets RAP1GAP and CDKN1C compared to expression of the corresponding proteins in a standard glioma population, can be diagnosed as having an aggressive form of glioma and can be predicted to have a shorter survival.

Significance is estimated based upon tumors with high expression (higher than 50% above the median of a standard glioma population versus tumors with low expression (less than 50% below the median of standard glioma population). By significantly higher or significantly lower expression of a protein in the group TCF12/HEB, RAP1GAP, CDKN1C, ID2, and ID3) is meant a difference of at least about 50% higher or 50% lower, respectively, from the median levels expressed in the standard glioma population (herein comprising 1,043 gliomas). The higher the level of ID2 and ID3, and the lower the levels of TCF12/HEB, RAP1GAP and CDKN1C, the more aggressive the glioma. By contrast, those gliomas that express the lowest levels of ID2 and ID3 and the highest levels of TCF12/HEB and its targets RAP1GAP and CDKN1C can be diagnosed as having the least aggressive form of glioma and are expected to live longer. Certain embodiments are directed to method for diagnosing the most and least aggressive forms of glioma according to these criteria.

The results of model-informed survival analysis described below, together with genetic and functional studies, establish that by preserving anchorage to the perivascular niche, ID activity is required for maintenance of mesenchymal high-grade glioma, specifically ID2 and ID3. These results support efforts to treat malignant brain gliomas by way of pharmacological inactivation of ID 2 and/or ID3 proteins, for example using inhibitory oligonucleotides or specific inhibitors of expression.

### 3. Background

### Id Proteins

The Id proteins are helix-loop-helix transcription factors that have been implicated in the control of cell differentiation (Norton et al., Id helix-loop-helix proteins in cell growth and differentiation. Trends Cell Biol., 8:58-65, 1998). In addition to this role in differentiation, Id proteins also have been implicated in cell-cycle control. In particular, the role of Id proteins as positive regulators of cell-cycle progression has been firmly established for one member of the Id family, Id2 (Norton supra; lavarone et al., The helix-loop-helix protein Id-2 enhances cell proliferation and binds to the retinoblastoma protein. Genes Dev., 8:1270-84, 1994; Lasorella et al., Id2 specifically alters regulation of the cell cycle by tumor suppressor proteins. Mol. Cell. Biol., 16:2570-78, 1996.). Only Id2, and not the other members of the Id-protein family (Id1 and Id3), is able to disrupt the anti-proliferative effects of tumor-suppressor proteins of the Rb family (i.e., the ‘pocket’ proteins: Rb, p107, and p130), thereby allowing cell-cycle progression. It is known that Id2 mRNA is overexpressed in neoplastic cells that give rise to pancreatic cancer (Kleef et al., The helix-loop-helix protein Id2 is overexpressed in human pancreatic cancer. Cancer Res., 58(17):3769-72, 1998). ID proteins are highly expressed in a large variety of human cancers (Nat Rev Cancer. 2005 August; 5(8):603-14. Id family of helix-loop-helix proteins in cancer. Perk J, Iavarone A, Benezra R.)

Id2 protein is highly expressed in cells of neuroblastomas and other solid pediatric tumors and it has been shown to mediate signaling by Myc oncoproteins, such that inhibition of Id2 in solid pediatric tumors has an anti-proliferative effect.

ID proteins are generally viewed as inhibitors of differentiation and enhancers of proliferation and stemness (9). However, they can exert different roles depending on the cellular context and the particular biological system (10). During normal development of the brain, ID proteins prevent premature cell fate determination and differentiation (11-13). Recently, we discovered that ID proteins preserve anchorage of NSCs to the extracellular niche microenvironment by repressing bHLH-mediated transcriptional activation of the gene coding for the RAP1-GTPase inhibitor Rap1GAP, thus precluding premature detachment of NSCs from the ventricular surface and initiation of differentiation (13). Accumulation of ID proteins is detected in a variety of tumor types including HGG in which the highest levels of ID proteins have been associated with the most aggressive form of the disease, the glioblastoma multiforme (GBM) (14). The redundant activity of ID proteins towards their intracellular targets (the bHLH transcriptional activators) suggests that combined inactivation of multiple Id genes may be required to uncover significant phenotypic changes (15). Expression of ID1 and ID3 has been associated with the tumor-initiating capacity of GICs and recent work has established that overexpression of ID proteins (Id3 and Id4) is sufficient to reprogram Ink4a/Arf−/− astrocytes to cells with GIC features (16-18). However, ablation of Id1 alone or in combination with Id3 has minimal effect on tumor growth and animal survival in mouse models of HGG displaying a proneural phenotype (19). Paradoxically, high levels of ID1 identify glioma cells with high self-renewal capacity but lower tumorigenic ability relative to ID1-low cells possessing limited self-renewal capacity. Accordingly, the high expression of ID1 in human HGG with a proneural phenotype is associated with more favorable clinical outcome (19).

Unless otherwise indicated, Id proteins and TCF12/HEB, RAP1GAP and CDKN1C proteins include both the particular protein and protein analogues thereof. The GenBank Accession Numbers for these proteins are set forth below:

TCF12 Transcription Factor 12 [Homo Sapiens]
Gene ID: 6938
Ensembl: ENSG00000140262
OMIM: 600480
UniProtKB: Q99081

### RAP1GAP RAP1 GTPase Activating Protein [Homo Sapiens]

Gene ID: 5909
Ensembl: ENSG00000076864
OMIM: 600278
UniProtKB: P47736
   CDKN1C cyclin-Dependent Kinase Inhibitor 1C (p57, Kip2) [Homo Sapiens]
Gene ID: 1028
Ensembl: ENSG00000129757
OMIM: 600856
UniProtKB: P49918

### ID2 Inhibitor of DNA Binding 2, Dominant Negative Helix-Loop-Helix Protein [Homo Sapiens]

Gene ID: 3398
Ensembl: ENSG00000115738
OMIM: 600386
UniProtKB: Q02363
   ID3 inhibitor of DNA Binding 3, Dominant Negative Helix-Loop-Helix Protein [Homo Sapiens]
Gene ID: 3399
Ensembl: ENSG00000117318
OMIM: 600277
UniProtKB: Q02535

The studies described herein were designed to address the significance of concurrent genetic inactivation of three Id genes (Id1, Id2, Id3) exclusively in brain tumor cells and temporally set after tumor initiation.

### 4. Summary of Methods

To make the vector, an IRES-Cre-ER cassette (also herein referred to as the Cre-recombinase feature or Cre-recombinase) was linked to cDNA encoding the oncogene HrasV12 and shRNA against the tumor suppressor Tp53 (hereafter shp53). “ER” means Estrogen Receptor; “IRES” means Internal Ribosomal Entry Site. Certain embodiments of the invention are directed to the herein-described lentivirus vectors. Other embodiments are directed to target cells or conditional animal knockout models that can be infected by the vectors, wherein the cells comprise floxed Id1 and Id2 and either do not express Id3 (Id3 −/−) such as the conditional Id null mice (Id1L/L; Id2L/L; Id3−/− mice, herein also referred to as the “Id-cTKO” mouse) or cells in which all three Id genes are floxed. Other embodiments are directed to animals comprising these cells (or cells isolated from the animals) that have been transduced with the described lentiviral vectors such as brain cells, more particularly glial cells, transduced with the HrasV12-Cre-ER-shp53 lentivirus vector.

In other embodiments the HrasV12-Cre-ER-shp53 lentivirus is modified by substituting one or more different oncogene (or proto-oncogene) for HrasV12, for example, another variant of RAS or WNT, MYC, ERK, and TRK. There are several systems for classifying oncogenes, but there is not yet a widely accepted standard. There are several categories that are commonly used:

| Category | Examples | Cancers | Gene functions |
|---|---|---|---|
| Growth factors, or | c-Sis | glioblastomas, | induces cell |
| mitogens | | fibrosarcomas, | proliferation. |
| | | osteosarcomas, breast | |
| | | carcinomas, and | |
| | | melanomas | |
| Receptor tyrosine | epidermal growth factor | Breast cancer, | transduce signals for |
| kinases | receptor (EGFR), | gastrointestinal stromal | cell growth and |
| | platelet-derived growth | tumours, non-small-cell | differentiation. |
| | factor receptor (PDGFR), | lung cancer and pancreatic | |
| | and vascular endothelial | cancer^{[19]} | |
| | growth factor receptor | | |
| | (VEGFR), HER2/neu | | |
| Cytoplasmic | Src-family, Syk-ZAP-70 | colorectal and breast | mediate the |
| tyrosine kinases | family, and BTK family | cancers, melanomas, | responses to, and the |
| | of tyrosine kinases, the | ovarian cancers, gastric | activation receptors |
| | Abl gene in CML - | cancers, head and neck | of cell proliferation, |
| | Philadelphia chromosome | cancers, pancreatice | migration, |
| | | cancer, lung cancer, brain | differentiation, and |
| | | cancers, and blood cancers | survival |
| Cytoplasmic | Raf kinase, and cyclin- | malignant melanoma, | Involved in organism |
| Serine/threonine | dependent kinases | papillary thyroid cancer, | development, cell |
| kinases and their | (through overexpression). | colorectal cancer, and | cycle regulation, cell |
| regulatory subunits | | ovarian cancer | proliferation, |
| | | | differentiation, cells |
| | | | survival, and |
| | | | apoptosis |
| Regulatory | Ras protein | adenocarcinomas of the | involved in signalling |
| GTPases | | pancreas and colon, | a major pathway |
| | | thyroid tumors, and | leading to cell |
| | | myeloid leukemia | proliferation. |
| Transcription | myc gene | malignant T-cell | They regulate |
| factors | | lymphomas and acute | transcription of genes |
| | | myleoid leukemias, breast | that induce cell |
| | | cancer, pancreatic cancer, | proliferation. |
| | | retinoblastoma, and small | |
| | | cell lung cancer | |

The oncogene-carrying vectors that further suppress expression of p53 described herein, will induce cancer in normal cells that carry floxed genes, such as Id1 and Id2 genes, in the absence of tamoxifen. Once cancer is established in the animal, tamoxifen can be administered to delete the folxed genes, in order to study the impact of the deletion on cancer progression. Thus, transduction with an appropriately modified lentivirus enables the creation of mammalian, preferably mouse, models that are of general use to study the effects of deletion of targeted genes on cancer. Embodiments of the modified gene delivery vehicles, such as lentivirus vectors, include the general formula: oncogene(s) (HRASV12, MYCN, EGFR)—Cre-ER such as IRES)—inhibitory p53 oligonucleotide (such as shp53). The order of the various elements in the gene delivery vehicle is arbitrary, for example the lentivirus could be Cre-ER-Oncogene-shp53, etc.

In the experiments described herein the role of Id genes in glioma was studied by deleting Id1, Id2 and Id3 selectively in glioma cells after the period of cancer initiation by the oncogene and p53 suppression. Floxed Id1 and Id2 genes were deleted by tamoxifen activation of Cre-recombinase in the HrasV12-Cre-ER-shp53 lentivirus-transduced glioma cells in the conditional Id null mice (Id1L/L; Id2L/L; Id3−/− mice, herein also referred to as the “Id-cTKO” mouse). The Id-null mouse was made by crossing mice harboring floxed alleles of Id1 (Id1L/L) and Id2 (Id2L/L) with constitutive Id3 knockout mice (Id3−/−) to generate Id1L; Id2L/L; Id3−/− (Id-cTKO) mice (13). Certain embodiments are directed to the new conditional Id1L/L; Id2L/L; Id3−/− mice Id-null mouse and to cells therefrom. Mice in which ID3 was floxed were also used.

In the experiments described herein, HrasV12-Cre-ER-shp53 lentiviral particles were injected in the hippocampus of four week-old Id-cTKO mice and tumor initiation/progression was examined before and after tamoxifen-activation of the Cre recombinase which resulted in deleting the floxed Id2 and Id3 genes making an ID depleted animal. Because the new model using the HrasV12-Cre-ER-shp53 lentivirus confines Id deletion selectively to targeted transduced glioma cells after tumor initiation, the confounding effects that might derive from Id deletion in other Id-expressing populations such as endothelial cells, tumor stroma or immune cells are avoided.

### 5. Summary of Experimental Results

1. Certain embodiments are directed to the lentiviral vector: HrasV12-Cre-ER-shp53 or pTomo-HrasV12-Cre-ER-shp53 (and the other herein described lentiviruses) which upon injection into a group of targeted cells selectively induces diffuse malignant tumor lesions. In the presence of tamoxifen which activates the Cre-ER, the expression of any floxed genes will be blocked due to the deletion of gene.

2. Tamoxifen treatment of glioma tumor-bearing Id null mice transduced with Id-Ctko/pTomo-HrasV12-Cre-ER-shp53_entiviral-treated mice caused first, a loss of ID1 and ID2 expression and reduced positivity for Ki67 in advanced tumors analyzed by immunofluorescence seven days after a single four-day cycle of tamoxifen treatment.

3. Tamoxifen-mediated ablation of the two floxed Id genes (Id 1 and Id 2) in Id3 −/− mice transduced with Id-Ctko/_pTomo-HrasV12-Cre-ER-shp53, did not impact retention of the tumor-initiating HrasV12 protein. Therefore, this mouse model allows selective and specific deletion of floxed Id 1 and id 2 genes in glioma cells in which the Id3 gene has been knocked out. The model can be adapted for any type of tumor by Administering the vector to a desired cell type, such as creating a nephroma by transducing kidney cells, etc.

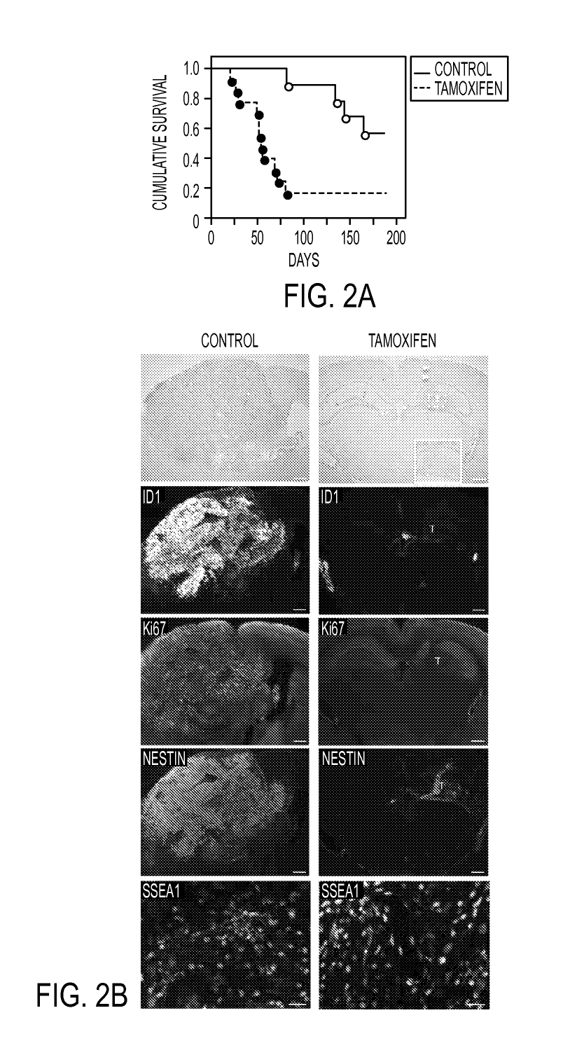

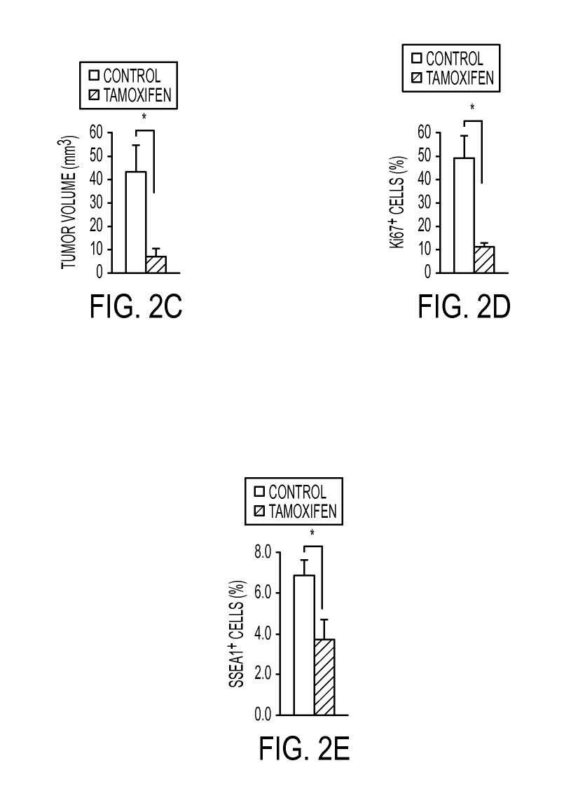

4. Tamoxifen-induced Id1 and Id 2 ablation in the Id mouse model that is Id3 −/− (Id-Ctko/transduced with pTomo-HrasV12-Cre-ER-shp53 (also herein referred to as Id deleted mice) resulted in significant extension of survival with 64% of the mice (seven of eleven) alive after an median of twenty weeks (p=0.002, FIG. 2A). By contrast eighty-five percent of mice (eleven of thirteen) wherein the Id genes were not ablated succumbed because of intracranial tumors within an median of ten weeks from lentiviral transduction. Tumor suppression was not merely an effect of tamoxifen.

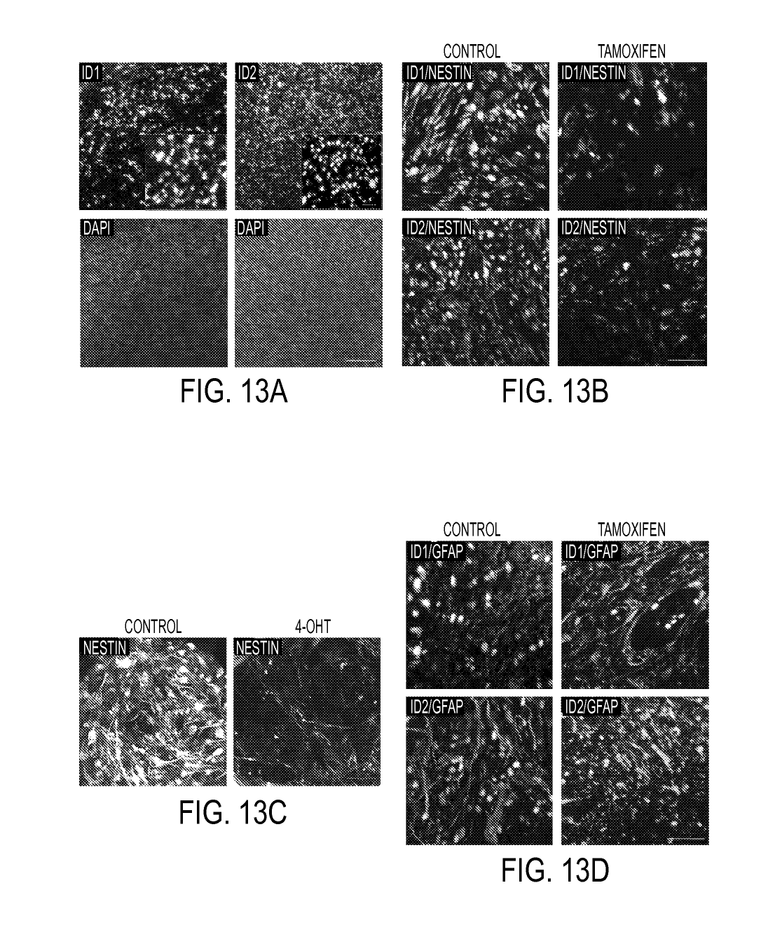

5. Glioma lesions in tamoxifen-induced Id ablation in the Id null mouse model (Id-Ctko transduced with pTomo-HrasV12-Cre-ER-shp53) showed a marked reduction in the proliferation rate (four-fold as measured by Ki67 immunoreactivity) and a striking decrease of the stem cell markers nestin and SSEA1 compared with control tumors (FIG. 2B-E and FIG. 13B). It was further shown that ID proteins play a key role in maintaining self-renewal and the tumorigenic capacity of GICs.

6. Lowering ID dosage (by reducing expression of the ID proteins, or deleting the ID genes) in GICs caused the disruption of the supportive interaction between GICs and endothelial cells in the perivascular niche. It was also discovered that the integrity of the glioma perivascular niche and glioma aggressiveness require RAP1 activity.

7. Gene expression profiles from four control (ID proficient) and four tamoxifen-treated (ID deficient, Id-cTKO) pTamo-HrasV12-Cre-ER-shp53 mouse gliomas were compared to a human glioma classifier dataset made up of 70 human glioma samples from ATLAS-TCGA that were reliably classified as proneural and mesenchymal using a linear discriminant analysis (LDA). The analysis revealed that gliomas from control (ID proficient) mice belong to the aggressive mesenchymal subclass (overall probability for mesenchymal classification: 0.995±0.005). Loss of Id genes did not significantly modify the tumor phenotype.

8. Knowledge-based pathway analysis applied to genes that were differentially expressed between control and Id-depleted tumors revealed regulation by ID proteins of functional gene categories linked to plasma membrane, extracellular matrix, cell-cell signaling, cell adhesion, etc. (Table 3). Specifically Rap1GAP mRNA was markedly elevated in HrasV12-Cre-ER-shp53 glioma following tamoxifen-mediated deletion of Id genes, while it is barely detectable in control tumors. Thus Id deletion restored the normal expression of Rap1GAP in glioma.

9. The displacement of GICs from the perivascular niche is implemented by derepression of Rap1GAP (due to ID deletion), with consequent inhibition of the activity of RAP1, a master regulator of cell adhesion. These effects lead to depletion of GICs and decline of glioma-initiating capacity. Id proteins repress Rap1GAP, which leads to activation of RAP1, thereby facilitating glioma adhesion. When ID proteins are not expressed Rap1GAP is de-repressed and RAP1 is inhibited thereby blocking adhesion and facilitating glioma tumor regression. This is consistent with the report by the inventors that ID proteins preserve anchorage of NSCs to the extracellular niche microenvironment by repressing bHLH-mediated transcriptional activation of the gene coding for the RAP1-GTPase inhibitor Rap1GAP, thus precluding premature detachment of NSCs from the ventricular surface and initiation of differentiation (13).

10. The frequency of glioma cells that self-renew as gliomaspheres was drastically decreased by enforced expression of Rap1GAP and the size of tumor spheres was also decreased.

11. The Rap1GAP gene ranked in the top 2% of down-regulated genes in GBM, correlating with increased tumorigenicity and adhesion of glioma cells to endothelial cells.

12. Proportional hazards regression analysis revealed the HGG subgroup that displays high activity of the ID2-ID3-TCF12/HEB-RAP1GAP-CDKN1C pathway is markedly enriched for glioma with a mesenchymal phenotype which has a poor survival compared to the proneural subgroup. Specifically, gliomas that have significantly elevated expression of ID2 and ID3, and significantly reduced expression of the bHLH transcription factor TCF12/HEB and its targets RAP1GAP and CDKN1C compared to a median level for each corresponding protein in a standard glioma population, can be diagnosed as having an aggressive form of glioma. The higher the level of ID2 and ID3 and the lower the levels of TCF12/HEB, RAP1GAP and CDKN1C, the more aggressive the glioma. For practical use in diagnosis, a subject glioma sample can be analyzed, for example by immunohistochemistry or per, to determine levels of protein expression. If ID2 and ID3 expression is detected and TCF12/HEB, RAP1GAP and CDKN1C is not detected, then the glioma can be classified as aggressive.

13. These results collectively support efforts to treat malignant gliomas by way of pharmacological inactivation of ID2 or ID3 or both, either through inhibition of gene expression (transcription and translation) or by blocking the activity of the encoded protein. Means of inhibiting the ID proteins are described below and in detail in U.S. Pat. No. 7,816,089.

### 6. Gene Delivery Vehicles for Use in Methods of Treatment

Gene delivery vehicles of certain embodiments refers to a construct which is capable of delivering, and, within some embodiments expressing, one or more gene(s) or nucleotide sequence(s) of interest in a host cell. Representative examples of such vehicles include viral vectors such as retroviral vectors such as lentiviruses.

Vectors for delivering nucleic acids can be viral, non-viral, or physical. See, for example, Rosenberg et al., Science, 242:1575-1578 (1988), and Wolff et al., Proc. Natl. Acad. Sci. USA 86:9011-9014 (1989). Discussion of methods and compositions for use in gene therapy include Eck et al., in Goodman & Gilman's The Pharmacological Basis of Therapeutics, Ninth Edition, Hardman et al., eds., McGray-Hill, New York, (1996), Chapter 5, pp. 77-101; Wilson, Clin. Exp. Immunol. 107 (Suppl. 1):31-32 (1997); Wivel et al., Hematology/Oncology Clinics of North America, Gene Therapy, S. L. Eck, ed., 12(3):483-501 (1998); Romano et al., Stem Cells, 18:19-39 (2000), and the references cited therein. U.S. Pat. No. 6,080,728 also provides a discussion of a wide variety of gene delivery methods and compositions. The routes of delivery include, for example, systemic administration and administration in situ. Well-known viral delivery techniques include the use of adenovirus, retrovirus, lentivirus, foamy virus, herpes simplex virus, and adeno-associated virus vectors.

### A. Viral Vectors

Viral vectors can also be used for transfection of a mammalian cell and introducing a polynucleotide into a genome. In an indirect method, viral vectors, carrying genetic information, are used to infect target cells removed from the body, and these cells are then re-implanted. Direct in vivo gene transfer into postnatal animals has been reported for formulations of DNA encapsulated in liposomes and DNA encapsulated in proteoliposomes containing viral envelope receptor proteins (Nicolau et al., Proc. Natl. Acad. Sci USA 80:1068-1072 (1983); Kaneda et al., Science 243:375-378 (1989); Mannino et al., Biotechniques 6:682-690 (1988). Viral vectors can be injected or transduced into host cells in vitro, which are then adoptively transferred and serve as delivery vehicles, such as T cells (Nakajima, A., et al., J. Clin. Invest., vol. 17(21), p. 1293-1310 (2001) and Tuohy, V. K., et al., J. Neuroimmunol., vol. 17(2), p. 226-32 (2000)), fibroblasts (Rabinovich, G. A., et al., J. Exp. Med., vol. 19, p. 385-98 (1999)), dendritic cells (DCs) (Kim, S. H., et al., J. Immunol., vol. 166(21), p. 3499-3550 (2001) and Morita, Y., et al., J. Clin. Invest., vol. 17(21), p. 1275-84 (2001)) and stem cells (ATCC or autolougous).

### B. Retroviral Vectors

Retroviral vectors are gene transfer plasmids wherein the heterologous nucleic acid resides between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764). Suitable retroviral vectors for use herein are described, for example, in U.S. Pat. Nos. 5,399,346 and 5,252,479; and in WIPO publications WO 92/07573, WO 90/06997, WO 89/05345, WO 92/05266 and WO 92/14829, which provide a description of methods for efficiently introducing nucleic acids into human cells using such retroviral vectors. Other retroviral vectors include, for example, mouse mammary tumor virus vectors (e.g., Shackleford et al., Proc. Natl. Acad. Sci. U.S.A. 85:9655-9659 (1998)), lentiviruses, and the like.

### C. Lentivirus Vectors

Lentiviruses are a subclass of Retroviruses. They have recently been adapted as gene delivery vehicles (vectors) thanks to their ability to integrate into the genome of non-dividing cells, which is the unique feature of Lentiviruses as other Retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides. The site of integration is unpredictable, which can pose a problem. The provirus can disturb the function of cellular genes and lead to activation of oncogenes promoting the development of cancer, which raises concerns for possible applications of lentiviruses in gene therapy. For safety reasons, lentiviral vectors never carry the genes required for their replication. To produce a lentivirus, several plasmids are transduced into a so-called packaging cell line, commonly HEK 293. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion.

In a preferred embodiment a gene delivery vehicle is a lentivirus vector comprising one or more oncogenes (e.g., RAS, WNT, MYC, ERK, and TRK), an IRES-Cre-ER cassette, and a gene encoding an oligonucleotide that inhibits p53 expression such as shp53. Transduction of normal brain cells in the Id null mouse with this newly discovered lentivirus vector recapitulates mesenchymal tumors that are the most aggressive subtype of HGG, and at the same time permits the temporally controlled tamoxifen-induced deletion of one or more floxed targeted genes (e.g., Id gene) exclusively in the glioma tumor following the period of cancer initiation. Deletion of three Id genes induced rapid release of glioma-initiating cells (GICs) from the perivascular niche followed by tumor regression, and (2) that the displacement of GICs from the perivascular niche is implemented by de-repression of Rap1GAP (as a result of Id gene deletion), with consequent inhibition of the activity of RAP1, a master regulator of cell adhesion. These effects lead to depletion of GICs and decline of glioma-initiating capacity.

Certain embodiments directed to the lentivirus vectors (HrasV12-Cre-shp53 and pTomo-HrasV12-Cre-ER-shp53 (and the other herein described lentiviruses and gene delivery vehicles). Upon injection into a group of targeted cells the vectors selectively induce localized malignant tumor lesions and permit the selective deletion from tumor cells of any floxed genes upon exposure to or contact with tamoxifen. Tamoxifen was discovered by pharmaceutical company Imperial Chemical Industries (now AstraZeneca) and is sold under the trade names Nolvadex, Istubal, and Valodex. Tamoxifen was approved by the FDA in December 1997. However, the drug, even before its patent expiration, was and still is widely referred to by its generic name “tamoxifen.” Tamoxifen is also used as a research tool to trigger tissue-specific gene expression in many conditional expression constructs in genetically modified animals including a version of the Cre-Lox recombination technique. Tamoxifen is available in 10 mg and 20 mg tablets and in solution: 10 mg/5 ml.

### 7. Inhibitory Oligonucleotides

In other embodiments, inhibitory oligonucleotides of any type that block p53 expression either at the gene or mRNA levels can be used. Some of the oligonucleotides include shRNAp53, siRNAp53, antisense blocking p53 expression and micoRNAs. P53-blocking oligonucleotides are well known in the art and are available from many sources commercially. The lentiviral vectors used in the Examples included a shRNA against p53.

Synonyms for p53 include: TP53 (BCCI, LFS1, P53, TRP53). Some examples of shRNAp53 include: AGTAGATTACCACTGGAGTC from the laboratory of Shinya Yamanaka, and GACTCCAGTGGTAATCTAC.

shp53 pLKO.1 puro vector from AddGene has the sequence 5′-CCGACTCCAGTGGTAATCTACTTCAAGAGAGTAGATTACCACTGGAGTCTTTTT.

Other p53 siRNA , shRNA and Lentiviral Particle Gene Silencers are available individually or as plasmids or lentiviral particles from Santa Cruz Biotechnology, Inc.:

| | | |
|---|---|---|
| p53 siRNA (h): | p53 shRNA Plasmid (h): | p53 shRNA (h) |
| sc-29435 | sc-29435-SH | Lentiviral Particles: sc- [symbol] |
| p53 siRNA | p53 shRNA Plasmid (h2): | p53 shRNA (h2) |
| (h2): sc-44218 | sc-44218-SH | Lentiviral Particles: sc- [symbol] |
| p53 siRNA | p53 shRNA Plasmid (m): | p5 shRNA (m) |
| (m): sc-29436 | sc-29436-SH | Lentiviral Particles: sc- [symbol] |
| p53 siRNA | p53 shRNA Plasmid (m2): | p53 shRNA (m2) |
| (m2): sc-44219 | sc-44219-SH | Lentiviral Particles: sc- [symbol] |
| P53 siRNA (r): | p53 shRNA Plasmid (r): | p53 shRNA (r) |
| sc-45917 | sc-45917-SH | Lentiviral Particles: sc- [symbol] |

[symbol]

indicates data missing or illegible when filed

A. shRNAs

ShRNAs as used herein, are well known in the art, and refer to small hairpin RNAs or short hairpin RNAs (shRNA) that are a sequence of RNA that makes a tight hairpin turn that can be used to silence target gene expression via RNA interference (RNAi). Expression of shRNA in cells is typically accomplished by delivery of plasmids or through viral or bacterial vectors. The shRNA hairpin structure is cleaved by the cellular machinery into siRNA. Small hairpin RNA (shRNA) is synonymous with short hairpin RNA. DNA encoding a shRNA is can be included on a plasmid and operably linked to a promoter. This plasmid can be introduced into cells in which inhibition of expression a target sequence is desired. This plasmid is usually passed on to daughter cells, enabling inheritance of the gene silencing. Once produced or present in a cell, the hairpin structure of shRNA is cleaved by cellular machinery into siRNA.

B. siRNAs

SiRNAs are RNA duplexes normally 16-30 nucleotides long that can associate with a cytoplasmic multi-protein complex known as RNAi-induced silencing complex (RISC). RISC loaded with siRNA mediates the degradation of homologous mRNA transcripts, therefore siRNA can be designed to knock down protein expression with high specificity. Unlike other antisense technologies, siRNA function through a natural mechanism evolved to control gene expression through non-coding RNA. This is generally considered to be the reason why their activity is more potent in vitro and in vivo than either antisense ODN or ribozymes. A variety of RNAi reagents, including siRNAs targeting clinically relevant targets, are currently under pharmaceutical development, as described, e.g., in de Fougerolles, A. et al., Nature Reviews 6:443-453 (2007).

While the first described RNAi molecules were RNA.RNA hybrids comprising both an RNA sense and an RNA antisense strand, it has also been demonstrated that DNA sense:RNA antisense hybrids, RNA sense:DNA anti-sense hybrids, and DNA:DNA hybrids are capable of mediating RNAi (Lamberton, J. S, and Christian, A. T., (2003) Molecular Biotechnology 24: 111-119). In addition, it is understood that RNAi molecules may be used and introduced to cells in a variety of forms. Accordingly, as used herein, RNAi molecules encompasses any and all molecules capable of inducing an RNAi response in cells, including, but not limited to, double-stranded oligonucleotides comprising two separate strands, i.e. a sense strand and an antisense strand, e.g., small interfering RNA (siRNA); double-stranded oligonucleotide comprising two separate strands that are linked together by non-nucleotidyl linker; oligonucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is at least 14, and in other embodiments at least 15, 20, 25, 29, 35, 40, or 50 nucleotides in length. In certain embodiments, it is less than 200, 100, or 60 nucleotides in length.

Hairpin siRNA compounds will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region. In certain embodiments, the overhangs are 2-3 nucleotides in length. In some embodiments, the overhang is at the sense side of the hairpin and in some embodiments on the antisense side of the hairpin.

A “double stranded siRNA compound” as used herein, is a siRNA compound which includes more than one, and in some cases two, strands in which interchain hybridization can form a region of duplex structure.

The antisense strand of a double stranded siRNA compound may be equal to or at least, 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length. As used herein, term “antisense strand” means the strand of a siRNA compound that is sufficiently complementary to a target molecule, e.g. a target RNA.

The sense strand of a double stranded siRNA com-pound may be equal to or at least 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The double strand portion of a double stranded siRNA compound may be equal to or at least, 14, 15, 16 17, 18, 19, 20, 21, 22, 23, 24, 25, 29, 40, or 60 nucleotide pairs in length. It may be equal to or less than 200, 100, or 50, nucleotides pairs in length. Ranges may be 15-30, 17to 23, 19 to 23, and 19 to 21 nucleotides pairs in length.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1-3 nucleotides. The overhangs can be the result of one strand being longer than the other, or the result of two strands of the same length being staggered. Some embodiments will have at least one 3′ overhang. In one embodiment, both ends of a siRNA molecule will have a 3′ overhang. In some embodiments, the overhang is 2 nucleotides.

The length for the duplexed region is between 15 and 30, or 18, 19, 20, 21, 22, and 23 nucleotides in length, e.g., in the ssiRNA compound range discussed above. ssiRNA compounds can resemble in length and structure the natural Dicer processed products from long dsiRNAs. Hairpin, or other single strand structures which provide the required double stranded region, and a 3′ over-hang are also within the invention.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an ssiRNA compound of 21 to 23 nucleotides.

A siRNA compound is “sufficiently complementary” to a target RNA, e.g., a target mRNA, such that the siRNA compound silences production of protein encoded by the target mRNA. In another embodiment, the siRNA compound is “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in certain embodiments, the siRNA compound specifically dis-criminates a single-nucleotide difference. In this case, the siRNA compound only mediates RNAi if exact complementary is found in the region (e.g., within 7 nucleotides of) the single-nucleotide difference.

### C. MicroRNAs

Micro RNAs (miRNAs) are a highly conserved class of small RNA molecules that are transcribed from DNA in the genomes of plants and animals, but are not translated into protein. Processed miRNAs are single stranded 17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also impli-cated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D 140-D144; “The microRNA Registry” Griffiths-Jones S, NAR, 2004, 32, Database Issue, D 109-D111.

### D. Antisense

In certain embodiments, a nucleic acid may be an antisense oligonucleotide directed to a target polynucleotide. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence, e.g. a target gene mRNA. Antisense oligonucleotides are thought to inhibit gene expression by binding to a complementary mRNA. Binding to the target mRNA can lead to inhibition of gene expression either by preventing translation of complementary mRNA strands by binding to it or by leading to degradation of the target mRNA Antisense DNA can be used to target a specific, complementary (coding or non-coding) RNA. If binding takes places this DNA/RNA hybrid can be degraded by the enzyme RNase H. In particular embodiment, antisense oligonucleotides contain from about 10 to about 50 nucleotides, more preferably about 15 to about 30 nucleotides. The term also encompasses antisense oligonucleotides that may not be exactly complementary to the desired target gene. Thus, the invention can be utilized in instances where non-target specific-activities are found with antisense, or where an antisense sequence containing one or more mismatches with the target sequence is the most preferred for a particular use.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. See for example (U.S. Pat. No. 5,739,119 and U.S. Pat. No. 5,759,829); (Jaskulski et al., Science. 1988 Jun. 10; 240(4858):1544-6; Vasanthakumar and Ahmed, Cancer Commun. 1989; 1(4):225-32; Penis et al., Brain Res Mol Brain Res. 1998 Jun. 15; 57(2):310-20; U.S. Pat. No. 5,801,154; U.S. Pat. No. 5,789,573; U.S. Pat. No. 5,718,709 and U.S. Pat. No. 5,610,288); (U.S. Pat. No. 5,747,470; U.S. Pat. No. 5,591,317 and U.S. Pat. No. 5,783,683).

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

### 8. Transduction of Cells

It is established in the art that transduction of cells is the process of transferring nucleic acid into a cell using a DNA or RNA virus. (See, eg., U.S. Patent Apn. No. 20130142764). Lentiviral vectors have the capacity to transduce cells, including non dividing cells, and are increasingly proposed for gene therapy. (See U.S. Patent Apn. No. 20130157357). The Lentiviridae subclass of retrovirus can infect most cell types including non-dividing cells. This property makes lentivirus attractive for gene therapy. Several replication-defective recombinant lentiviral vectors have already been constructed by different groups (Naldini PNAS 93, 11382-8, Science, 1996). These reengineered and detoxified lentiviral vectors are proposed as the most efficient and safe gene therapy vectors (Zufferey R, & Kim V. N. J Virol, 72, 9873-80, 1998). In certain embodiments, cells such as glioma cells and Id-null cells (Id1L/L; Id2L/L; Id3−/−) are transduced with the gene delivery vehicle (e.g., the lentivirus vector HrasV12-Cre-ER-shp53). The glioma cell comprises at least one floxed gene such as Id1, Id2, and Id3. In other embodiments, an animal comprises one or more floxed genes, transduced with the gene delivery vehicle (e.g., the lentivirus vector HrasV12-Cre-ER-shp53).

### 9. Transgenic Knockout Mice

Mouse models of human cancers have been instructional in understanding the basic principles of cancer biology. Three major types of animal models: xenografts, human tumor tissues or cell lines transplanted in immunodeficient mice; transgenesis, transgenic mice containing oncogenes with tissue-specific expression; and genetic knockouts, transgenic mice in whom a gene, usually a suppressor gene, is in the heterozygous state or is fully deleted are known in the art. Additional modifications to these methods, such as conditional knock-ins and knockouts, have become useful tools to study initiation, maintenance and progression of a wide variety of neoplasias. The mouse as a genetic model has been greatly enhanced by transgenic and knockout technologies, which have allowed for the study of the effects of the directed over-expression or deletion of specific genes and are well know in the art (see, e.g., Jones, et al., “Generation and functional confirmation of a conditional null PPAR gamma allele in mice.” Genesis. 2002 February; 32(2):134-7 and Zhuo, L. et al. “hGFAP-Cre transgenic mice for manipulation of glial and neuronal function in vivo.” Genesis 31, 85-94 (2001).

Certain embodiments may be directed to a conditional Id-null mouse (Id1L/L; Id2L/L; Id3−/−). In these embodiments, the Id-null mouse is made by crossing mice harboring floxed alleles of Id1 (Id1L/L) and Id2 (Id2L/L) with constitutive Id3 knockout mice (Id3−/−) to generate Id1L/L; Id2L/L; Id3−/− (Id-cTKO) mice. In this floxed mouse, loxP sites flanked the entire protein-coding region of the Id2 gene. These mice were crossed with Id1L/L and Id3−/− or Id3L/L to generate IdcTKO mice (See, also Guo Z, Li H, Han M, Xu T, Wu X, Zhuang Y. “Modeling Sjogren's syndrome with Id3 conditional knockout mice.” Immunol Lett. 2011; 135(1-2):34-42 and Pan L, Sato S, Frederick J P, Sun X H, Zhuang Y. “Impaired immune responses and B-cell proliferation in mice lacking the Id3 gene.” Mol Cell Biol. 1999; 19(9):5969-5980.)

In certain embodiments, methods are provided for (a) obtaining a transgenic animal comprising one or more floxed genes; (b) obtaining the gene delivery vehicle (e.g., HrasV12-Cre-ER-shp53), comprising an oncogene, IRES-Cre-ER cassette, and a gene encoding an oligonucleotide that inhibits p53; (c) transducing cells in a target area of the animal with the gene delivery vehicle; (d) waiting a period of time sufficient for cancerous cells to form in the target area; (e) contacting the cancerous cells with tamoxifen in an amount sufficient to activate the IRES-Cre-ER cassette thereby deleting one more floxed genes; and (f) determining an effect of deleting the one more floxed alleles in the cancerous cells. The effect may be (i) a slowing of the growth rate of the cancerous cell, (ii) a slowing of the rate of metastasis, and (iii) lengthening survival.

### 10. Methods of Diagnosis and Treatment

### A. Diagnosis

According to a method of the present invention, the diagnostic sample, such as a sample of a glioma, from a subject may be assayed in vitro or in vivo. In accordance with the present invention, where the assay is performed in vitro, a diagnostic sample from the subject may be removed using standard procedures. The diagnostic sample of a glioma is a sample on which standard assays to analyze nucleic acids and proteins can be run. It has been further discovered that together with four key components of the ID-bHLH transcriptional network in neural stem cells (specifically TCF12/HEB, ID2, ID3, CDKN1C), RAP1GAP contributes to a “five-gene signature set” that segregates two sub-groups of glioma patients with markedly divergent clinical outcomes. Gliomas that have significantly elevated expression of ID2 and ID3, and significantly reduced expression of the bHLH transcription factor TCF12/HEB and its targets RAP1GAP and CDKN1C compared to standard glioma population, can be diagnosed as having an aggressive form of glioma. Significance is estimated based upon tumors with high expression (higher than 50% above the median of a standard glioma population or reference group) versus tumors with low expression (less than 50% below the median of a standard glioma population). By significantly elevated or significantly reduced expression of a protein is meant a difference of at least about 50% up or down, respectively, from the median levels expressed in all glioma in the group. The higher the level of ID2 and ID3 and the lower the levels of TCF12/HEB, RAP1GAP and CDKN1C, the more aggressive the glioma. By contrast, those gliomas that express the lowest levels of ID2 and ID3 and the highest levels of TCF12/HEB and its targets RAP1GAP and CDKN1C can be diagnosed as having the least aggressive form of glioma and are expected to live longer. The standard glioma population for the Examples herein regarding expression profiles and clinical information constituted 1,043 newly diagnosed patients with HGG (20, 38-40). The platform for all 4 data sets was Affymetrix based and used 2 different chip types: U95Av2 and U133A. Microarray data sets were batch normalized using a previously described method (52). Following batch normalization, the median value of the bHLH transcription factors was calculated and the data for each sample were categorized as “0” (if below the median) or “1” (if at or above the median). Since the direction of the survival association was opposite between the bHLH transcription factors and targets compared with the ID genes, the ID gene expression was characterized as “1” if it was below the median and “0” if it was above the median. Gene combinations were then added, with equal weightings for each gene, for each sample. Proportional hazards regression analysis with the 5-gene signature was used to calculate the effect of the integrated tumor score on survival.

Certain embodiments are directed to methods for diagnosing the most and least aggressive forms of glioma according to the above criteria. These methods comprise (a) obtaining a sample of glioma in a subject; (b) determining a level of expression of each protein selected from the group consisting of TCF12/HEB, RAP1GAP, CDKN1C, ID2 and ID3 in the subject glioma sample, (c) comparing the level of expression of each protein in the subject glioma sample to a known median level of expression of each of the corresponding proteins TCF12/HEB, RAP1GAP, CDKN1C, ID2 and ID3 in a standard glioma population, and (d) if the level of expression of TCF12/HEB, RAP1GAP and CDKN1C is significantly lower in a subject glioma sample compared to the known median for each corresponding protein in the standard glioma population, and the level of each of ID2 and ID3 expression is significantly higher in the subject glioma sample than the standard glioma population, then the glioma is diagnosed as an aggressive glioma having very poor prognosis. In each of these embodiments, it is then possible to (d) treat the aggressive glioma in a subject in need thereof. The level of expression of each of the proteins TCF12/HEB, RAP1GAP, CDKN1C, ID2 and ID3 in the subject glioma sample is determined by a method selected from the group consisting of determining the level of each of the proteins, or the level of cDNA for each respective protein, or the level of mRNA encoding each respective protein in the subject glioma sample. Immumohistochemistry and PCR are typically used for these measurements.

The opposite is also true, so in other embodiments, if the level of expression of TCF12/HEB, RAP1GAP and CDKN1C is significantly higher in the subject glioma sample compared to the known median level of expression of each of the corresponding proteins in the standard glioma population, and the level of each of ID2 and ID3 expression is significantly lower in the subject glioma sample than the standard glioma population then it is possible to diagnose the glioma as a non-aggressive glioma carrying a better prognosis. In each of these embodiments, it is then possible to treat the non-aggressive glioma in a subject in need thereof. The level of expression for the encoded protein, cDNA, or mRNA may be measured in the subject glioma sample.

As used herein, “expression” means the transcription of a gene, for example an ID gene or Rap1Gap or Rap1 into at least one mRNA transcript, or the translation of at least one mRNA into the encoded protein, as defined above. Accordingly, a diagnostic sample may be assayed for gene expression by assaying for the encoded protein, cDNA, or mRNA. In these embodiments, the level of expression is determined using immunohistochemistry or PCR.

Other methods are provided in certain embodiments where a sample of a glioma is obtained from a subject. The level of expression of each of the proteins TCF12/HEB, RAP1GAP, CDKN1C, ID2, and ID3 is then determined in the glioma sample. If expression of TC12/HEB, RAP1GAP, and CDKN1C cannot be detected in the glioma sample, and if expression of ID2 and ID3 is detectable in the glioma sample, then the glioma is diagnosed as an aggressive glioma, and treatment of the aggressive glioma in the subject may follow. The opposite is also true. If expression of TC12/HEB, RAP1GAP, and CDKN1C can be detected in the glioma sample, and if expression of ID2 and ID3 is un-detectable in the glioma sample, then the glioma is diagnosed as a non-aggressive glioma, and treatment of the non-aggressive glioma in the subject may follow.

In accordance with the methods of the present invention, a diagnostic sample of a subject's glioma (herein also a “glioma sample”) may be assayed for gene expression using assays and detection methods readily determined from the known art, including, without limitation, immunological techniques, hybridization analysis, fluorescence imaging techniques, and/or radiation detection. For example, according to the method of the present invention, a diagnostic sample of the subject may be assayed for expression using an agent reactive with the targeted gene or encoded protein. As used herein, “reactive” means the agent has affinity for, binds to, or is directed against the targeted gene or protein compared to other proteins. As further used herein, an “agent” shall include a protein, polypeptide, peptide, nucleic acid (including DNA or RNA), antibody, Fab fragment, F(ab′)₂ fragment, molecule, compound, antibiotic, drug, and any combinations thereof. A Fab fragment is a univalent antigen-binding fragment of an antibody, which is produced by papain digestion. A F(ab′)₂ fragment is a divalent antigen-binding fragment of an antibody, which is produced by pepsin digestion. Preferably, the agent of the present invention is labeled with a detectable marker. Antibodies include polyclonal or monoclonal forms.

Alternatively, a diagnostic sample of a subject may be assayed for gene expression using hybridization analysis of nucleic acid extracted from the diagnostic sample taken from the subject. Indeed this is described in the Examples using microarrays that comprise oligonucleotides that are sufficiently complementary to and hybridize with mRNA transcribed from the five-gene signature set. According to this method of the present invention, the hybridization analysis may be conducted using Northern blot analysis of mRNA. This method also may be conducted by performing a Southern blot analysis of DNA using one or more nucleic acid probes which hybridize to nucleic acid encoding the target protein. The nucleic acid probes may be prepared by a variety of techniques known to those skilled in the art, including, without limitation, the following: restriction enzyme digestion of the target nucleic acid; and automated synthesis of oligonucleotides having sequences which correspond to selected portions of the nucleotide sequence of the targeted nucleic acid, using commercially-available oligonucleotide synthesizers, such as the Applied Biosystems Model 392 DNA/RNA synthesizer.

The nucleic acid probes used in the present embodiments to detect particular mRNAs may be DNA or RNA, and may vary in length from about 8 nucleotides to the entire length of the targeted nucleic acid. The nucleic acid used in the probes may be derived from mammals. The nucleotide sequence for human ID proteins (Id2 and Id3), TCF12/HEB, RAP1GAP and CDKN1C are known and are set forth herein. Using these sequences as a probe, the skilled artisan could readily clone corresponding cDNA from other species. In some embodiments mRNA expression used an Affymetrix-based assay and used 2 different chip types: U95Av2 and U133A. Microarrays (or microchips) to which are adhered oligonucleotides that selectively hybridize with the mRNA encoding ID2, Id3), TCF12/HEB, RAP1GAP and CDKN1C are commercially available. In addition, the nucleic acid probes of the present invention may be labeled with one or more detectable markers. Labeling of the nucleic acid probes may be accomplished using one of a number of methods known in the art—e.g., nick translation, end labeling, fill-in end labeling, polynucleotide kinase exchange reaction, random priming, or SP6 polymerase (for riboprobe preparation)—along with one of a variety of labels—e.g., radioactive labels, such as ³⁵S, ³²P, or ³H, or nonradioactive labels, such as biotin, fluorescein (FITC), acridine, cholesterol, or carboxy-X-rhodamine (ROX). Combinations of two or more nucleic acid probes (or primers), corresponding to different or overlapping regions of any targeted nucleic acid, also may be used to assay a diagnostic sample using, for example, PCR or RT-PCR.

The detection of protein expression in the embodiments of the present invention may be followed by an assay to measure or quantify the extent of expression in a diagnostic sample of a subject. Such assays are well known to one of skill in the art, and may include immunohistochemistry/immunocytochemistry, flow cytometry, mass spectroscopy, Western blot analysis, or an ELISA for measuring amounts of protein. For example, to use an immunohistochemistry assay, histological (paraffin-embedded) sections of tissue may be placed on slides, and then incubated with an antibody against the protein of interest. The slides then may be incubated with a second antibody (against the primary antibody), which is tagged to a dye or other calorimetric system (e.g., a fluorochrome, a radioactive agent, or an agent having high electron-scanning capacity), to permit visualization of the protein present in the sections.

### B. Treatment

Treatments for both non-aggressive gliomas and aggressive gliomas are known in the art. The best treatment for an individual patient takes into account the tumor location, potential symptoms, and potential benefits versus risks of the different treatment options (modalities). Treatment for a glioma is customized to the individual patient and may include surgery, radiation therapy, chemotherapy, or observation. Surgery is the most common initial treatment for gliomas. A biopsy taken during surgery provides tissue samples to the pathologist, who will then be able to make an accurate diagnosis of the tumor's composition, which is critical to getting the best treatment. Surgery can also allow for the removal of tumor tissue to relieve pressure in the brain caused by the tumor. This often needs to be done on an urgent basis. Radiation therapy and chemotherapy usually follow surgery once the diagnosis or name of the tumor is determined. These treatments are called adjuvant treatments.

| Radiation therapy for gliomas: | Chemotherapy for gliomas: |
|---|---|
| Radiation therapy is performed | Chemotherapy, including GLIADEL ® |
| after surgery for high-grade | wafers and targeted therapy, is |
| gliomas. It is also used to | recommended for some high-grade |
| treat gliomas in locations | gliomas after surgery and radiation |
| where surgery is not safe | therapy. |
| and for recurrent gliomas. | |
| Three types of radiation therapy | Three types of chemotherapy may be |
| are used to treat gliomas: | used to treat glioma: |
| Internal radiation with the | Systemic, or standard, |
| GliaSite Radiation Therapy | chemotherapy |
| System | |
| External beam radiation therapy | GLIADEL ® wafers |
| Stereotactic radiosurgery | Targeted therapy |

Drug options include Temozolomide (brand names Temodar and Temodal and Temcad) which is an oral chemotherapy drug. It is an alkylating agent used for the treatment of Grade IV astrocytoma—an aggressive brain tumor, also known as glioblastoma multiforme—as well as for treating melanoma, a form of skin cancer. Temozolomide is also indicated for relapsed Grade III anaplastic astrocytoma and not indicated for, but as of 2011 used to treat oligodendroglioma brain tumors in some countries, replacing the older (and less well tolerated) PCV (Procarbazine-Lomustine-Vincristine) regimen.

After treatment, brain scans may show brain tissue that looks like glioma. This is often dead tissue or changes in healthy tissue caused by radiation therapy, chemotherapy or both. Neurosurgeons and neuroradiologists will closely monitor this to determine whether the glioma has recurred. If so, neurosurgeons can perform another surgical procedure.

As discussed above, certain embodiments are directed to methods for diagnosing the most aggressive and least aggressive forms of glioma. Although clinically treated the same, it is important to distinguish non-aggressive forms of glioma and aggressive forms of glioma for several reasons including the psychology of the patient and determination of the best possible treatment options. These methods of diagnosis as described are important, but the ultimate goal is to manipulate tumor cells using an anti-Id therapy to reduce expression of the ID proteins or delete the Id genes so that an “Id-less” tumor results ultimately decreasing its malignancy.

These therapies include identifying chemical compounds (e.g., small molecules) that inhibit Id 1, Id2, and Id3 with the use of chemical compounds that have the ability to cross the blood-brain barrier and penetrate the tumor. Those of skill in the art would know how to dose these chemical compounds. The goal is to treat the patient's cancer, and in doing so higher doses of chemical compounds be necessary to eliminate or control the cancer. Ultimately, any side effects at higher doses may be further managed. If the cancer is relatively nonaggressive it may respond to a lower dose than would be needed for treating aggressive forms of gliomas.

### 11. EXAMPLES

### Example 1

### Materials and Methods

### Animals

The Id2L/L mouse was used to generate conditional Id2 knock-out mice and has been described (13). In this floxed mouse, loxP sites flanked the entire protein-coding region of the Id2 gene. These mice were crossed with Id1L/L and Id3−/− or Id3L/L to generate IdcTKO mice (12, 48, 49).

### Histology and Immunostaining

Tissue preparation and immunohistochemistry on brain tumors and immunofluorescence staining were performed as previously described (26, 50, 51). Antibodies used in immunostaining are listed in Table 5. In histograms, values represent the mean values; error bars are standard deviations (SD) or standard error of the mean (SEM) as indicated in Figure legends. Statistical significance was determined by t test (two-tailed) using GraphPad Prism 4.0 software (GraphPad Inc., San Diego, Calif.). Quantification of the IL-6 intensity staining in was performed using NIH Image J software (http://rsb.info.nih.gov/ij/). The histogram of the intensity of fluorescence of each point of a representative field for each condition was generated. The fluorescence intensity of three fields from three independent tumors was scored, and standardized to the number of cells in the field.

### Cell Culture Conditions and Cell Culture Based Assays

Human embryonic kidney 293T and bEnd3 cells (ATCC) were grown in DMEM containing 10% Fetal Bovine Serum (FBS, Invitrogen). GBM-derived GICs were grown as spheres in Neurobasal media containing N2 and B27 supplements, and human recombinant FGF-2 and EGF (50 ng/ml each; Peprotech). EGFRvIII-shp53 iGICs were obtained by infecting Id1L/L; Id2L/L; Id3L/L astrocytes with EGFRvIII-Cre-ER-shp53 lentivirus. After infection, cells were cultured in Neurobasal media (Invitrogen) containing N2 and B27 supplements (Invitrogen), and human recombinant FGF-2 and EGF (50 ng/ml each).

For adhesion assay of pLOC-GFP or pLOC-RAP1-G12V; Q63E-GFP EGFRvIII-Cre-ER-shp53 iGICs spheres were generated by plating cells in low attachment plates (Corning). Cells were treated for 96 h with 500 nM tamoxifen or vehicle and an equal number of iGSC spheres (200 per 18 mm coverslip) were plated on bEND3 cells that had been cultured on cover slips for 36 h in iGSC defined medium. After 24 h, cultures were washed with PBS-1% BSA four times, fixed in 4% PFA for 10 min and examined by fluorescent microscopy. The number of GFP-positive spheres was scored by scanning the entire coverslip. Triplicate samples for each infection and treatment were analyzed and data are presented as the percentage of plated spheres. In histograms values represent the mean values; error bars are standard deviations.

Adhesion of pLOC-GFP, pLOC-Rap1GAP-GFP or pLOC-p27^{Kip1}-GFP transduced human GICs to endothelial cells was performed by plating bEnd3 cells on 18 mm cover slips and allow them to adapt to GSC medium for 36 h. Dissociated pLOC-GFP, pLOC-Rap1GAP-GFP or pLOC-p27^{Kip1}-GFP transduced glioma spheres were plated at a density of 25,000 cells/coverslip in quadruplicates. After 30 min, plates were vigorously washed with PBS-0.1% BSA 4 times to remove non-adherent cells. Cells were fixed with 4% PFA for 10 min, stained with anti-GFP antibody to identify GFP positive, lentivirus infected GICs and the number of GFP positive cells per field was scored. At least 2,000 GFP-positive cells were counted in each coverslip by scanning multiple fields. Results are presented as the mean±SD of quadruplicates samples. The assay was repeated twice.

For BrdU incorporation analysis, GICs transduced with pLOC-GFP, pLOC-Rap1GAP-GFP or pLOC-p27^{Kip1}-GFP were plated on cover slips and cultured in the presence of 10 μM BrdU for 2 hours. Cells fixed in 4% PFA were stained with anti-BrdU antibody and the number of BrdU positive cells was scored as a percentage of the total number of cells counterstained with DAPI.

For human and mouse glioma sphere formation, cells were infected with lentiviral particles. Three days later single cells were plated at density of ≦3 cells/well in triplicate in low attachment 96 well plates. The number and the size of spheres were scored after 10-14 days. Limiting dilution assay was performed as described previously (24). Spheres were dissociated into single cells and plated in 96-well plates in 0.2 ml of medium containing growth factors. Cultures were left undisturbed for 10 days, and then the percentage of wells not containing spheres for each cell dilution was calculated and plotted against the number of cells per well. Linear regression lines were plotted, and the number of cells required to generate at least one sphere in every well (=the stem cell frequency) was calculated. The experiment was repeated twice.

### Lentiviral Production

pLKO.1 lentiviral expression vectors carrying shRNAs were purchased from Sigma. The hairpin sequence targeting the Rap1GAP gene is CCTGGTATTCTCGCTCAAGTA. pLOC-GFP lentiviral expression vectors carrying RAP1A or Rap1GAP cDNA were purchased from Open Biosystems. The RAP1-G12V; Q63E mutant was generated using the Phusion Site Direct Mutagenesis kit (New England Biolabs). Lentivirus preparation and infections were performed as described (26).

### Clinical Outcome Analysis

Expression profiles and clinical information of 1,043 newly diagnosed HGG patients from the datasets were analyzed (20, 38-40). This 1,043 collective group is herein referred to as “the standard glioma population.” The platform for all 4 data sets was Affymetrix-based and used 2 different chip types: U95Av2 and U133A. Microarray datasets were batch-normalized using a previously described method (52). Following batch normalization, we calculated the median value of the bHLH transcription factors and categorized the data for each sample as “0” (if below the median) or “1” (if at or above the median). Since the direction of the survival association was opposite between the bHLH transcription factors and targets vs. the ID genes, we categorized the ID gene expression as “1” if it was below the median and “0” if it was above the median. Gene combinations were then added, (with equal weightings for each gene), for each sample. Proportional hazards regression analysis with the five-gene signature was used to calculate the effect of the integrated tumor score on survival.

### Biochemical Methods

The levels of active GTP-bound RAP1 were determined using the Active RAP1 Pull-Down and Detection Kit (Pierce) according to the manufacturer's instruction. Proteins were analyzed by immunoblotting using an anti-RAP1 antibody. RAP1 activity in Id1L/L; Id2L/L; Id3L/L astrocytes transformed by the expression of EGFRvIII-Cre-ER-shp53 and transduced with a pLOC-GFP-RAP1AG12V; Q63E or pLOC-vector-GFP lentivirus was measured as described above.

RNA Preparation and Real-Time Quantitative PCR (qRT-PCR)

RNA preparation and qRT-PCR were performed as described (26, 51). Primers used in qRT-PCR are listed in Table 6. The relative amount of specific mRNA was normalized to 18S. Results are presented as the mean±SD of triplicate amplifications.

### Intracranial Injections, Tamoxifen Treatment and Tumor Volume Evaluations

Intracranial injection of Ras-V12-IRES-Cre-ER-shp53 lentivirus was performed in 4-week-old Id-cTKO mice in accordance with guidelines of the International Agency for Research on Cancer's Animal Care and Use Committee. Briefly, 1.3 μl of purified lentiviral particles in PBS (1×10⁹/ml) were injected 1.45 mm lateral and 1.6 mm anterior to the bregma, and 2.3 mm below the skull using a stereotaxic frame (Kopf Instruments). Orthotopic implantation of mouse glioma cells was performed as described using 50,000 cells in 2 μl of phosphate buffer (26). Tamoxifen was administered using a feeding needle for 4 days at 9 mg/40 g of mouse weight, starting 12 days after surgery and at bi-weekly intervals thereafter for 3 additional cycles. Mice were monitored daily and sacrificed when neurological symptoms appeared. Tumor volume was obtained using three-dimensional measurements in the formula for an ellipsoid: [Length×Width ×Height×(π/6)]. The dimensions were derived from a complete, H&E stained, histological sectioning of the mouse brain. The length was determined by counting the number of sections containing tumor cells and multiplying it by the section thickness. The width and height measurements were taken from the section that showed the largest tumor area (53). Kaplan-Meier survival curve was generated using the DNA Statview software package (AbacusConcepts, Berkeley, Calif.).

### Mouse Tumor Microarrays and Computational Analyses

Total RNA was extracted from quadruplicate samples of mouse HRasV12-shp53-Cre-ER-IdcTKO induced tumors treated with tamoxifen or vehicle and used for analysis on Illumina MouseRef-8 v2.0 expression BeadChip. The raw array data was normalized using the Bioconductor package Lumi using quantile normalization. In order to classify mouse samples according to the human GBM phenotypes we used data obtained from The Cancer Genome Atlas Data Portal (54). Description of TCGA data, platforms, and analyses are available at http://tcga-data.nci.nih.gov/. The specific data sources were (according to Data Levels and Data Types) as follows: Expression data: “Level 2” normalized signals per probe set (Affymetrix HT_HG-U133A). First, we analyzed data to select samples that could be categorized as mesenchymal or proneural according to three published GBM phenotypic signatures (20, 26, 27). Differentially expressed genes were selected based on fold change ≧2 and p-value >10⁻⁵. Using these stringent criteria we identified 29 proneural and 41 mesenchymal samples. Then, we applied the R package (55) to the raw array data to normalize mouse and human data and remove batch effects. Finally, we applied the linear discriminant analysis (LDA) implemented in the MASS package (56) to classify the mouse samples. LDA is a well-established method to obtain a reduced-dimension representation of the data. LDA computes an optimal transformation (projection) by minimizing the within-class distance and maximizing the between-class distance simultaneously, thus achieving maximum class discrimination. In our study, class labels are available (proneural and mesenchymal) and supervised approaches such as LDA are more effective than unsupervised ones such as principal component analysis (PCA) for classification. In this analysis the 70 human GBM samples were treated as the training set. LDA returns a value ranging between 0 (not belonging) and 1 (belonging) to each sample in a data set, indicating the membership in each class. Thus, the values returned provide an indication of the likelihood of a sample belonging to each class. Each mouse tumor was then allocated to the class to which it most belongs. Probability equal to 1 indicates the maximum probability. The complement of the probability (1−p) can be interpreted as a p-value of the test to indicate how data are consistent with the null hypothesis (the sample does not belong to the predicted sub-group). Significant functional annotation clusters enriched in differentially expressed genes between control and tamoxifen-treated HRasV12-shp53-Cre-ER-IdcTKO tumors were predicted using DAVID (Database for Annotation, Visualization and Integrated Discovery, Bioinformatics Resources at the National Institute of Allergy and Infectious Diseases, NIH). The microarray expression data have been deposited in the ArrayExpress database (accession number: E-MTAB-1303).

### Statistics

Results are expressed as Mean±SD or Mean±SEM as indicated in Figure legends for the indicated number of observations. Statistical significance was determined by the unpaired 2-tailed Student's t test using GraphPad Prism 4.0 software (GraphPad Inc., San Diego, Calif.). P values are indicated in Figure Legends.

### Study Approval

All animal studies were reviewed and approved by the IACUC at Columbia University.

### Example 2

### Mouse Model Allowing Selective and Specific Deletion of Id Genes in Glioma Cells

A new mouse model of malignant glioma was designed to ask whether the consequences of Id deletion on tumor growth affect the cell-intrinsic properties of GICs including the competence to adhere to the perivascular niche. In the new model, Id deletion was selectively targeted to glioma cells so that after tumor initiation the glioma could be studied without the confounding effects that might derive from Id deletion in other Id-expressing population such as endothelial cells, tumor stroma or immune cells. NSCs in the hippocampus, a neurogenic area of the adult brain, were transduced with a lentivirus that expresses oncogenic ras (HrasV12) and shRNA against the tumor suppressor Tp53 (shp53) that generates HGG (21). Although oncogenic mutations affecting ras genes are uncommon in human GBM, Ras is frequently activated in HGG by aberrant signaling from multiple receptor tyrosine kinases (22). When the same lentiviral vector was tested expressing GFP instead of HrasV12 to identify infected cells, most of the GFP-positive cells co-stained with the radial glia and astrocytic marker GFAP, and were negative for the neuronal marker NeuN (FIG. 11A).

In order to temporally control deletion of Id selectively in tumor cells, an IRES-Cre-ER cassette was linked to HrasV12 cDNA and shp53, thus achieving Cre-recombinase activation by tamoxifen in vivo in the mouse. FIG. 11B is a schematic representation of pTomo-H-RasV12-IRES-Cre-ER-shp53 lentiviral vector. Activation of Cre recombinase by tamoxifen results in deletion of the floxed Id genes in Id null mice (Id-cTKO) exclusively in tumor cells.

To create a conditional Id-null mouse, mice harboring floxed alleles of Id1 (Id1L/L) and Id2 (Id2L/L) were crossed with constitutive Id3 knockout mice (Id3−/−) to generate Id1L/L; Id2L/L; Id3−/− (Id-cTKO) mice (13). pTomo-HrasV12-Cre-ER-shp53 lentiviral particles were injected in the hippocampus of four week-old Id-cTKO mice and tumor initiation/progression was examined.

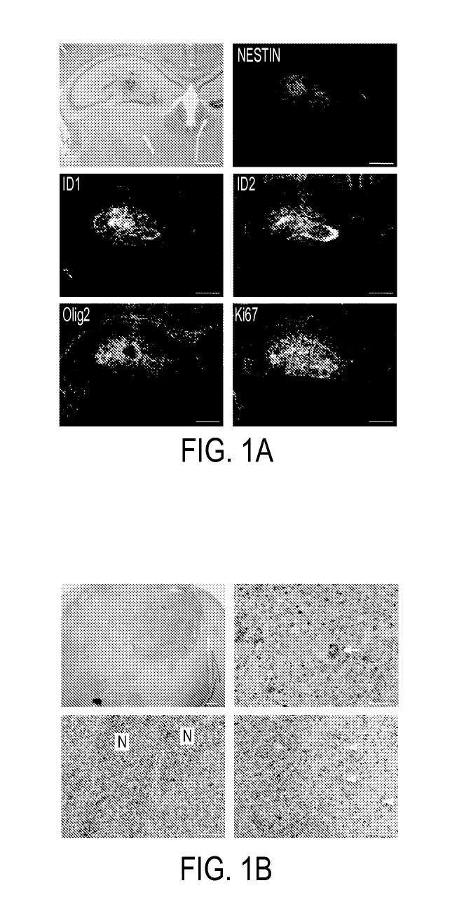

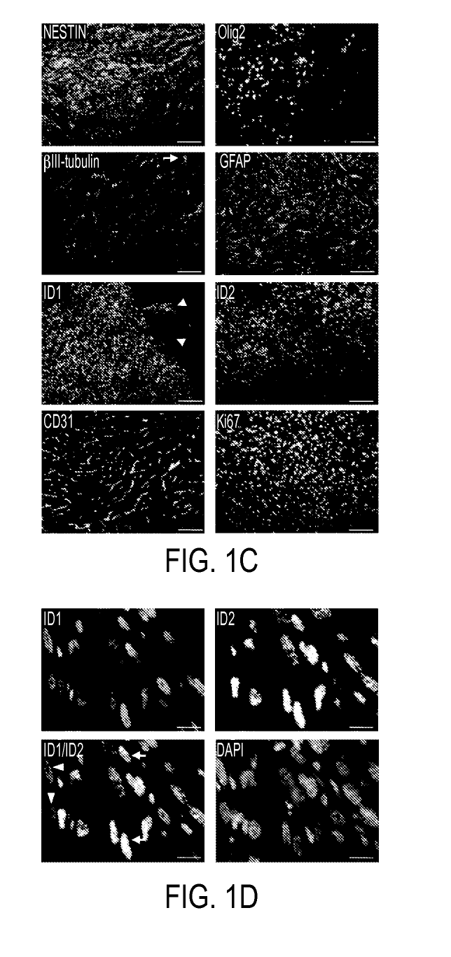

Definite tumor lesions were detected as early as twelve days after lentiviral transduction in 86% of the Id-cTKO control mice infected mice (six of seven, FIG. 1A, upper panels). At this stage, tumors in these four week old mice consisted of highly proliferative, Ki67⁺ cells showing reactivated expression of ID1 and ID2 (largely absent in the adult hippocampus), and robust positivity for nestin and Oligodendrocyte Transcription Factor 2 (Olig2) (FIG. 1A and FIG. 11C). Advanced tumors analyzed at the time of mouse euthanasia manifested features of HGG such as multinucleated giant cells, necrosis and pseudo-palisades, mitotic figures and propensity to invade the normal brain (FIG. 1B). Tumors remained strongly positive for ID1, ID2, Olig2, and nestin, expressed Glial Fibrillary Acidic Protein (GFAP) and included individual entrapped βIII-tubulin-positive neurons. The high positivity for Ki67 and the endothelial marker CD31 were indicative of rapid growth and rampant tumor angiogenesis, respectively (FIG. 1C). Together, these elements reflect those found in the human counterpart of the disease (glioma grade III-IV). Interestingly, double immunostaining experiments for ID1 and ID2 showed that the two ID proteins are frequently co-expressed in glioma cells (FIG. 1D).

To ascertain the therapeutic potential of Id deletion in brain tumors, tumor-bearing Id-Ctko/pTomo-HrasV12-Cre-ER-shp53 lentiviral-injected mice were treated with tamoxifen or vehicle (corn oil). First, a loss of ID1 and ID2 expression and reduced positivity for Ki67 was seen in advanced tumors analyzed by immunofluorescence seven days after a single four-day cycle of tamoxifen treatment (FIG. 12A-C). The residual cells staining positive for ID1 (but not ID2) in tamoxifen-treated gliomas were for the vast majority tumor endothelial cells, as shown by the nuclear morphology and co-staining for CD31 (FIG. 12A, B and data not shown). Glioma cells that stained positive for the HrasV12 oncoprotein also expressed ID proteins. However, the expression of HrasV12 was unaffected by Id deletion (FIG. 12D). This finding indicates that the tamoxifen-mediated ablation of Id genes in glioma cells does not impact retention of the tumor initiating HrasV12 protein. Therefore, this mouse model allows selective and specific deletion of Id genes in glioma cells.

### Example 3

### Suppression of ID Function Impacts Tumor Maintenance of HGG and Extends Survivals

Survival in tumor-bearing Id-cTKO confirm mice treated with tamoxifen or vehicle was evaluated twelve days after lentiviral transduction and at bi-weekly intervals thereafter. Eighty-five percent of oil-treated mice (eleven of thirteen) succumbed because of intracranial tumors within an average of ten weeks from lentiviral transduction. However, Id ablation with tamoxifen resulted in significant extension of survival with 64% of the mice (seven of eleven) alive after an median of twenty weeks (p=0.002, FIG. 2A). Tumor suppression was not merely an effect of tamoxifen, as tamoxifen treatment of wild type animals infected with HrasV12-Cre-ER-shp53 lentivirus (without ID ablation) did not affect tumor growth (data not shown). The tumors that eventually developed and caused death of tamoxifen-treated mice accumulated high levels of ID1 and ID2, thus showing that Id expression by glioma cells is required for tumor growth in this model (FIG. 13A).

### Example 4

### ID Protein Preserve GICs and are Required for Association of GICs with Endothelial Cells

Tumor-bearing Id-cTKO controls and tamoxifen-treated mice were sacrificed six weeks after tumor initiation. The majority of tumors (80%) in the tamoxifen-treated cohort displayed dramatic reduction of tumor volume compared with carrier-treated tumors and consisted of clusters of tumor cells confined to the hippocampus that had not progressed relative to tumors detected twelve days after viral infection (FIG. 2B-C, also compare FIG. 2B with FIG. 1A). As expected, these tumors either lacked or expressed negligible levels of ID proteins. Glioma lesions in Id deleted mice (treated with tamoxifen) showed a marked reduction in the proliferation rate (four-fold as measured by Ki67 immunoreactivity) and a striking decrease of the stem cell markers nestin and SSEA1 compared with control tumors (FIG. 2B-E and FIG. 13B). Expression of nestin in tamoxifen treated tumors was lower at this stage than in the controls at the earliest time point (compare FIG. 2B with FIG. 1A), suggesting that nestin down-regulation was independent of tumor size and specifically implemented by Id loss. The decrease of nestin expression was recapitulated in vitro when tumor explants established from HrasV12-Cre-ER-shp53 glioma bearing Id-cTKO mice were cultured in the presence of tamoxifen (FIG. 13C). In contrast, expression of GFAP was higher in tamoxifen treated tumors compared with controls (FIG. 13D).

### Example 5

### ID Proteins Play a Key role in Maintaining Self-Renewal and the Tumorigenic Capacity of GICs

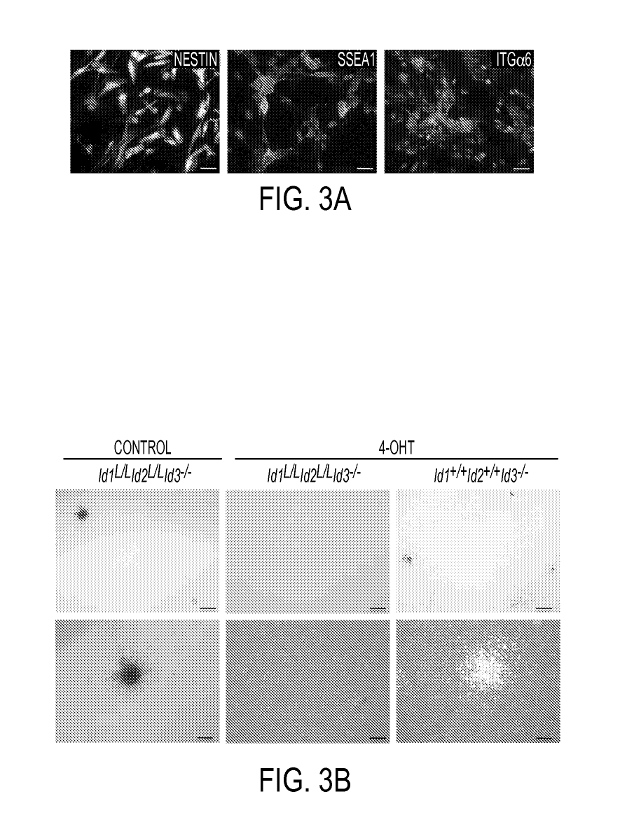

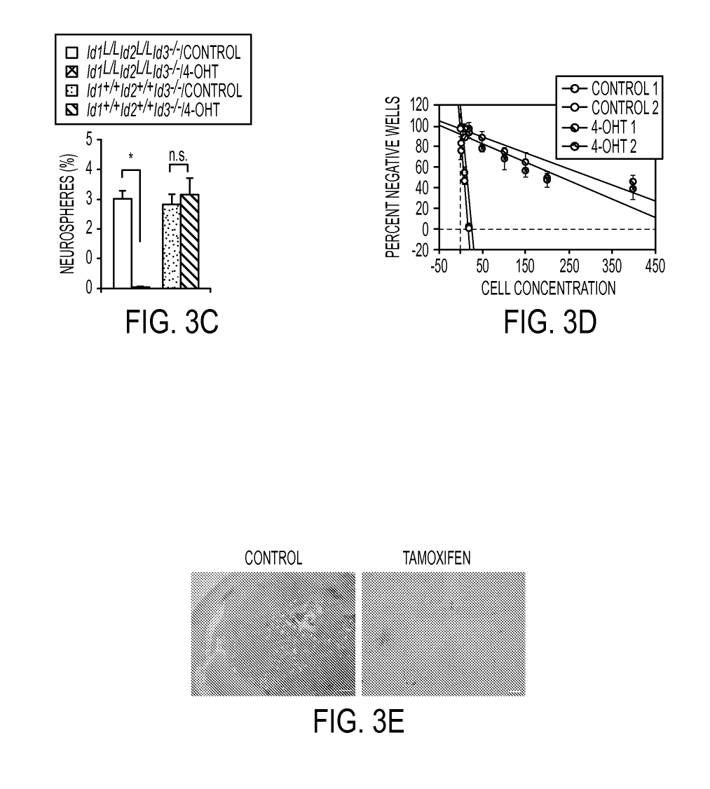

To analyze the consequences of Id ablation on GICs-enriched populations, cells were isolated from HrasV12-Cre-ER-shp53 glioma. GICs were selected based upon their ability to self-renew as tumor spheres in stem cell-permissive culture conditions (23), retain expression of the NSC and GIC markers SSEA1, integrin-α6 and nestin and display robust glioma-initiating capacity when orthotopically transplanted into the brain of immunodeficient mice (FIG. 3A-E). Treatment of GICs isolated from Id-cTKO mice with tamoxifen abolished the tumor-sphere forming capacity but the same treatment had no effect on GICs derived from Id1wt/wt; Id2wt/wt; Id3−/− mice (FIG. 3B, C). To accurately determine the frequency of glioma cells that self-renew and generate spheres within the glioma cell population, limiting dilution analysis was performed using cells from freshly dissociated tumors (24). Based on the Poisson distribution and the intersect at the 37% level, the minimal frequency of glioma cells endowed with stem cell capacity was estimated to be 0.29%±0.02 and 3.84%±0.51 in tumors derived from tamoxifen treated animals and control, respectively (p=0.006) (FIG. 3D). Finally, following orthotopic implantation of HrasV12-Cre-ER-shp53-Id-cTKO GICs into the brain of immunodeficient mice, tamoxifen efficiently blocked tumor formation (0 of 5 mice developed tumors) whereas highly aggressive glial tumors developed in the control group (5 of 5 mice developed tumors, FIG. 3E). The concordant phenotypes in vivo and in vitro, as well as across multiple experimental systems, support the view that ID proteins play a key role in maintaining self-renewal and the tumorigenic capacity of GICs.

### Example 6

### Id Impacts the Interaction between GICs and Endothelial Cells in the Tumor Perivascular Niche

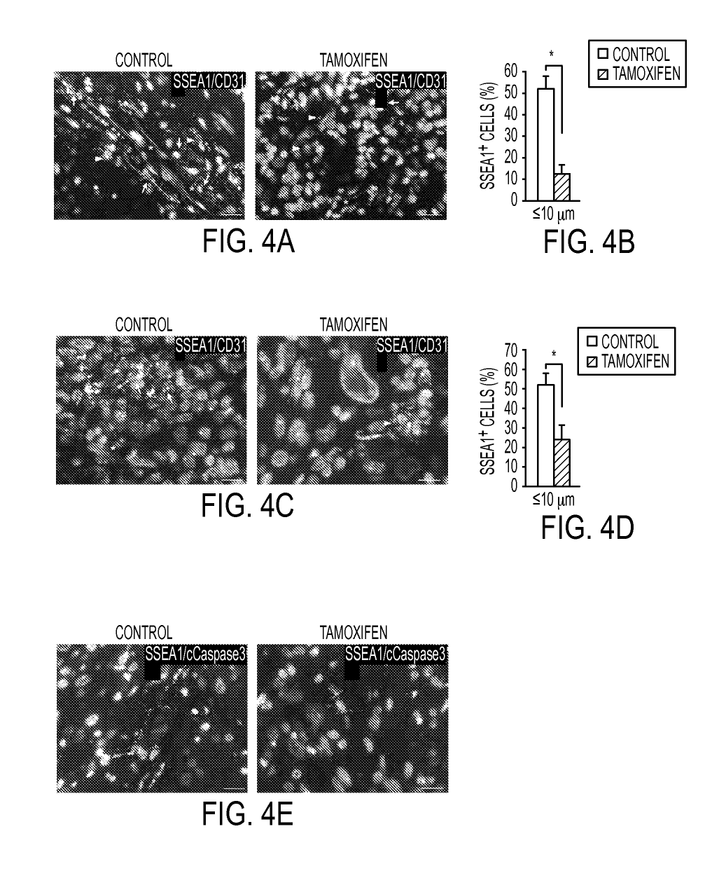

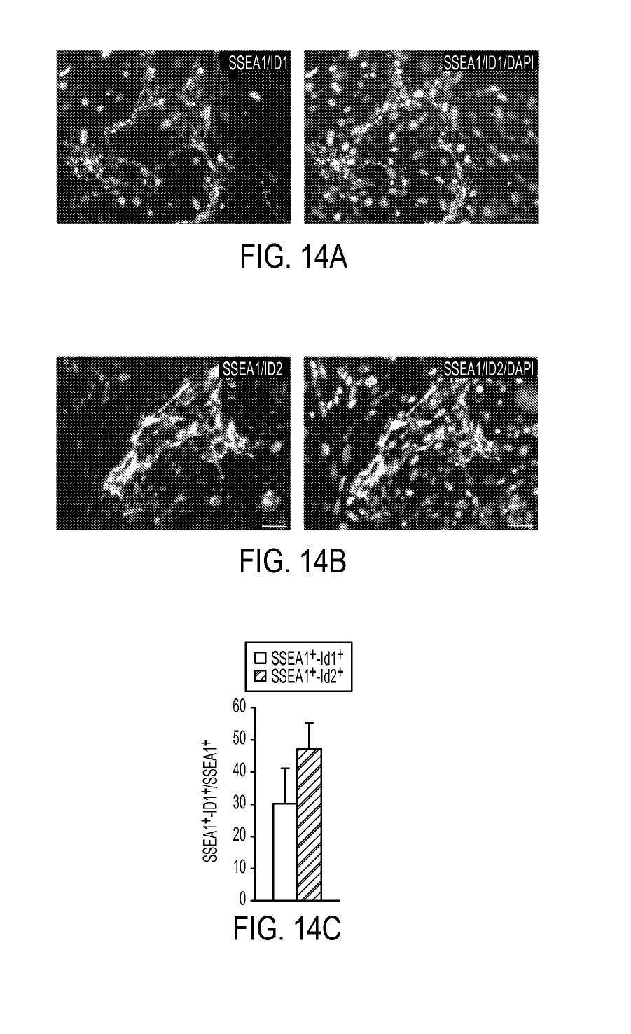

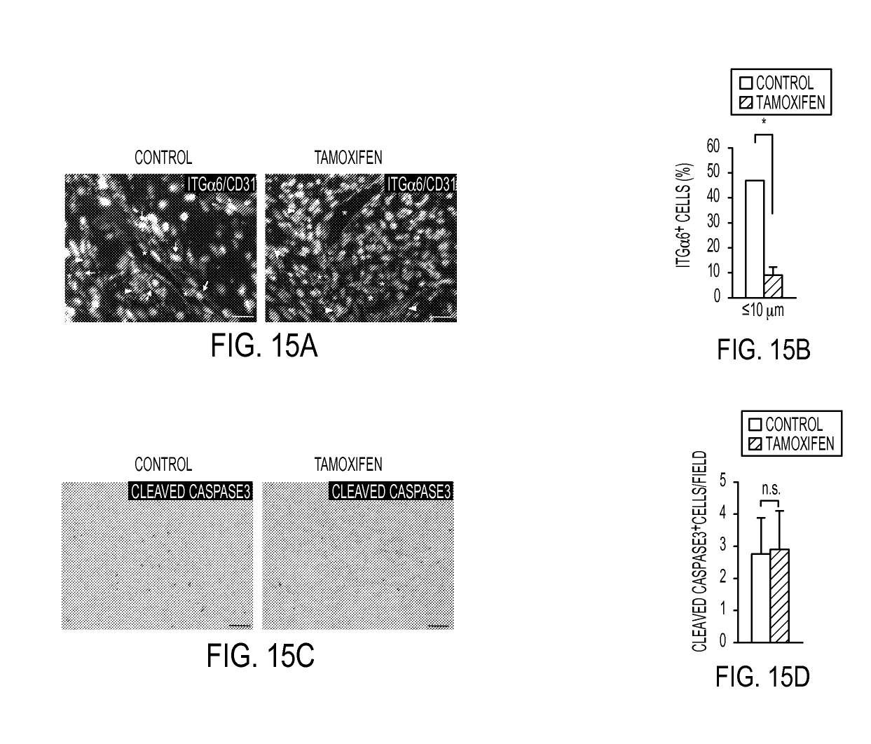

Loss of Id impacts the interaction between GICs and endothelial cells in the tumor perivascular niche. First, it was determined that a significant fraction of SSEA1-positive tumor cells were also positive for ID immunostaining, thus indicating that GICs express ID proteins in vivo (FIG. 14). Then, it was tested whether deletion of Id genes in GICs from established tumors influenced the residency of GICs in the perivascular niche. To examine the spatial relationship between GICs and endothelial cells in tumor blood vessels, the fraction of GICs in close proximity to CD31 positive endothelial cells (≦10 μm) was quantified in tamoxifen and vehicle treated tumors. GICs were independently identified using SSEA1 and integrin-α6 (ITGα6), two well-characterized GICs markers previously assigned to GICs in the perivascular niche (6, 25). Compared with controls, tumors carrying deletion of Id genes had a significantly reduced (five-fold) fraction of GICs residing within 10 μm from CD31-positive cells (FIG. 4A, B, FIG. 15A, B). This effect was detectable as early as seven days after treatment with tamoxifen without changes of cell survival measured either in the entire glioma tumor mass or selectively in the SSEA1-positive population of GICs (FIG. 4C-E, FIG. 15C, D). These findings indicate that a direct consequence of lowering ID dosage in GICs is the disruption of the supportive interaction between GICs and endothelial cells in the perivascular niche, and this effect is implemented without impairing cell survival.

### Example 7

### Integrity of the Glioma Perivascular Niche and Glioma Aggressiveness Require RAP1 Activity

To elucidate the molecular phenotype of HrasV12-Cre-ER-shp53 glioma and the changes triggered by Id ablation in glioma cells, gene expression profiles were obtained of HrasV12-Cre-ER-shp53 tumors in Id-cTKO mice treated with tamoxifen or vehicle for six weeks. Several studies have identified gene expression subgroups in human malignant glioma with the two most robust and alternative categories defined as proneural and mesenchymal and linked to better and worse clinical outcome, respectively (20, 26, 27). The goal was to determine whether the HrasV12-Cre-ER-shp53 mouse glioma in Id-Ctko tamoxifen-treated mice display a global gene expression profile that resembles one of the two key human glioma subtypes.

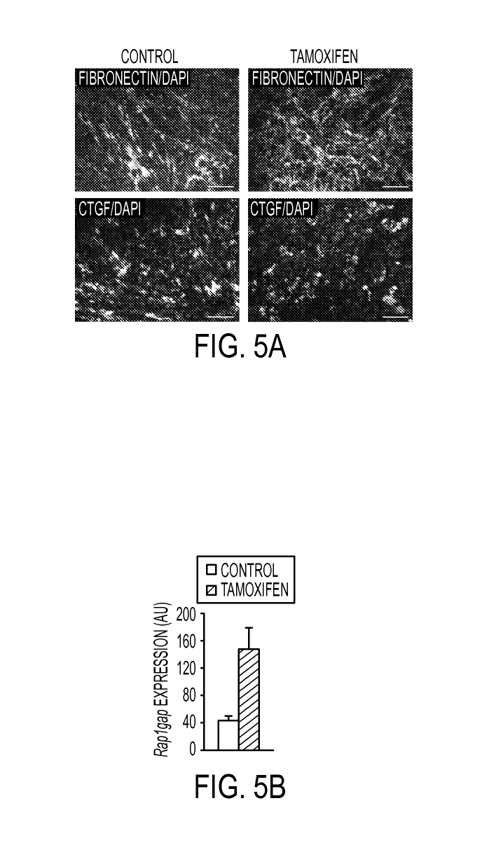

A training set of 70 human glioma samples from ATLAS-TCGA were generated that were reliably classified as proneural and mesenchymal and then applied a linear discriminant analysis (LDA) to compare the expression profiles from four control (ID proficient) and four tamoxifen-treated (ID deficient, Id-cTKO) HrasV12-Cre-ER-shp53 mouse gliomas to the human glioma classifier dataset (see Methods). The analysis revealed that glioma from control (ID proficient) mice belong to the mesenchymal subclass (overall probability for mesenchymal classification: 0.995±0.005). Loss of Id genes did not significantly modify the tumor phenotype although two of the four tamoxifen-treated tumors could not be classified as mesenchymal since they had slightly moved towards the proneural class (overall probability for mesenchymal classification: 0.895±0.11) (Table 1). This finding was corroborated by the robust positivity of the tumor cells for fibronectin and Connective Tissue Growth Factor (CTGF), two markers of mesenchymal GBM that did not show significant changes after Id deletion (26, 28) (FIG. 5A).

However, knowledge-based pathway analysis applied to the genes differentially expressed between control and Id-depleted tumors revealed regulation by ID proteins of functional gene categories linked to plasma membrane, extracellular matrix, cell-cell signaling, cell adhesion, etc. (Table 3). These categories are very similar to those acutely perturbed by Id deletion in NSCs through direct regulation of the bHLH target gene Rap1GAP, the inhibitor of RAP1-GTPase (13). Interestingly, Rap1GAP mRNA was markedly elevated in HrasV12-Cre-ER-shp53 glioma following tamoxifen-mediated deletion of Id genes (FIG. 5B).

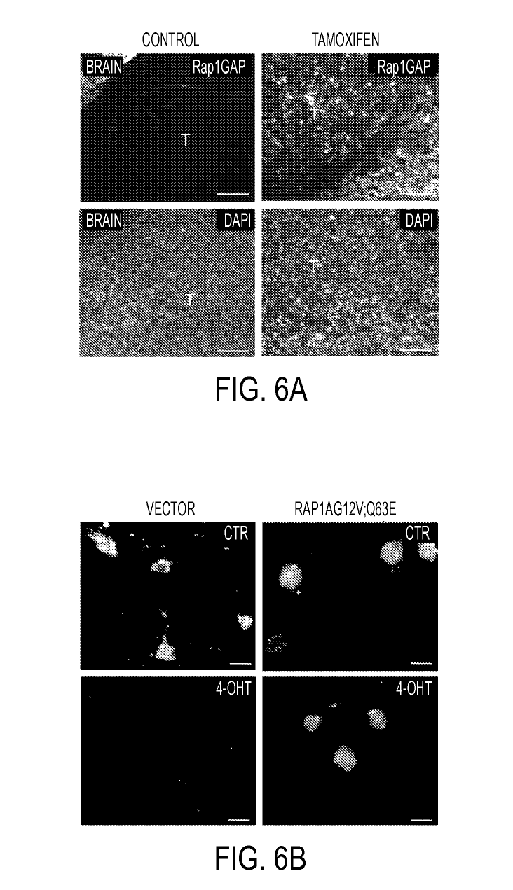

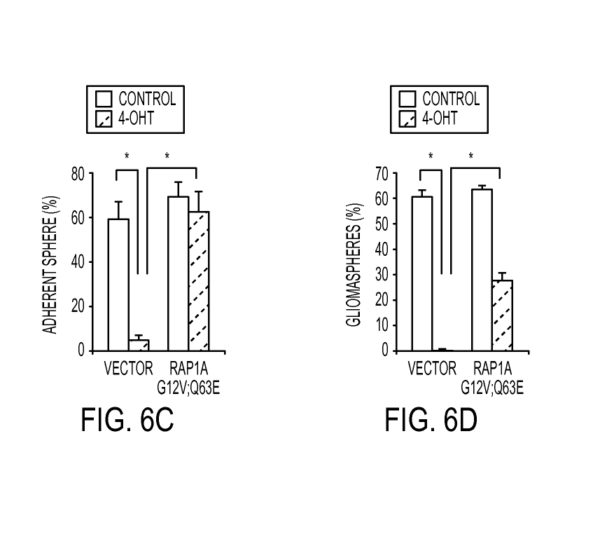

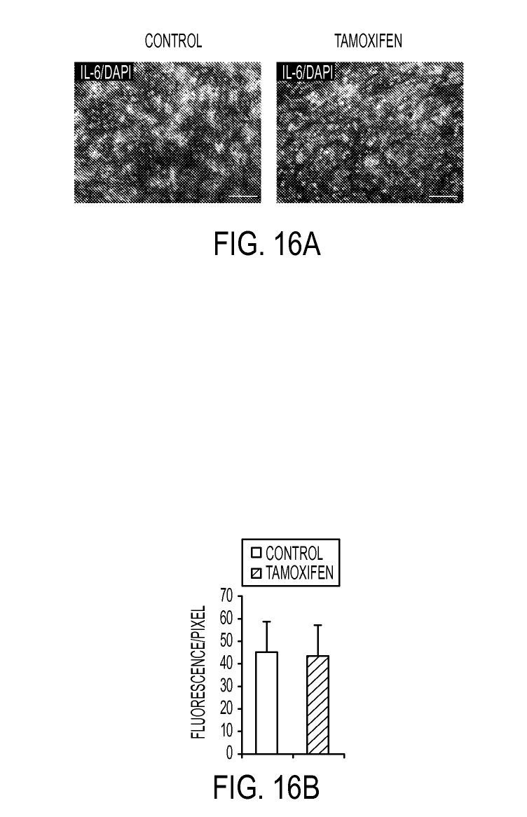

Depending on the cellular contexts, RAP1 can act as a key regulator of cell-cell, cell-ECM adhesion or both. The function of RAP1 for cell-ECM adhesion is implemented through its ability to operate as intracellular mediator of integrin signaling (29-32). Recently, integrin signaling emerged as an essential determinant for the anchorage of GICs to the perivascular brain tumor niche and tumor aggressiveness (6). Therefore, experiments were designed to test whether the adhesion defect manifested by Id-depleted GICs depends on the de-repression of Rap1GAP and consequent inhibition of RAP1-GTPase. First, it was established that Rap1GAP was expressed in the normal mouse brain but was barely detectable in control tumors (FIG. 6A). In tumors from tamoxifen-treated mice, the Rap1GAP protein was de-repressed to levels comparable to those found in the normal brain (FIG. 6A). These effects occurred without changes of the expression of interleukin-6 (IL-6), a cytokine abundantly expressed in this glioma model (FIG. 16) (18).

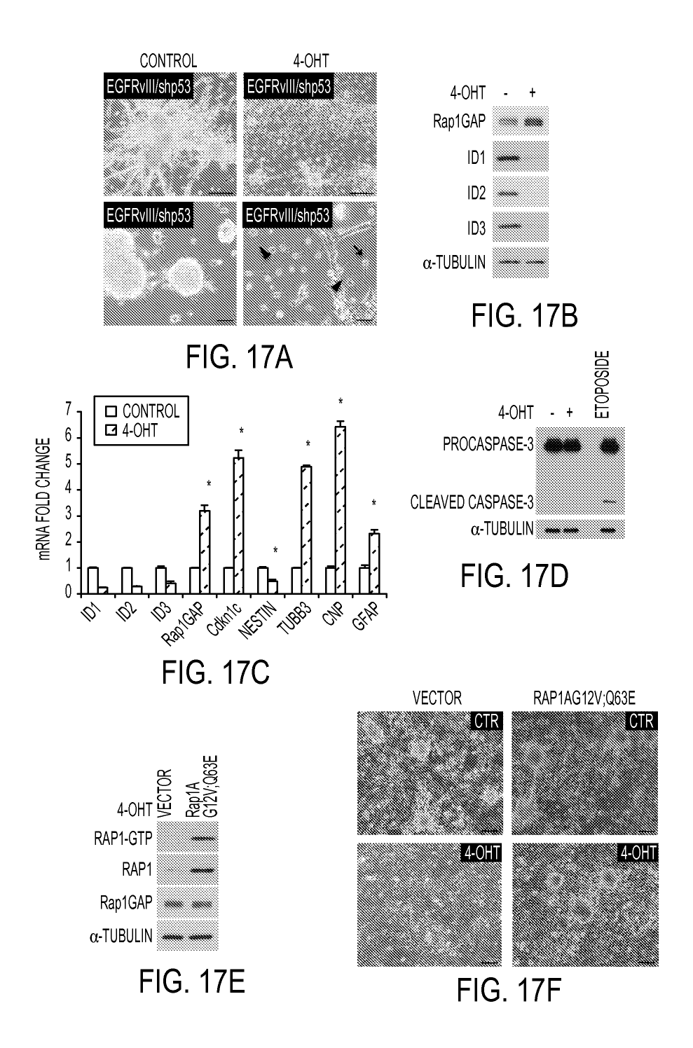

Having determined that Id deletion restored the normal expression of Rap1GAP in glioma, the role of Rap1GAP in GICs was examined and determined whether RAP1 is the significant target of Rap1GAP activity. To do this, a widely used cellular system developed to study the mechanistic events determining the glioma stem cell state was used. In this system, primary astrocytes are reprogrammed to a transformed stem cell phenotype (induced glioma stem cells, iGICs) by the expression of a mutant form of the Epidermal Growth Factor Receptor (EGFR), EGFRvIII concurrently with silencing of key tumor suppressor genes frequently deleted in human glioma (33, 34). Astrocytes derived from Id1L/L; Id2L/L; Id3L/L mice were engineered to express EGFRvIII-IRES-CreER and shp53, and treated the resulting neurospheres with vehicle or tamoxifen to delete Id genes. Loss of Id in iGICs induced the bHLH-ID targets Rap1GAP and Cdkn1c (13, 35), and led to morphological and molecular changes indicative of multi-lineage neural differentiation (FIG. 17A-C). These effects occurred in the absence of signs of apoptosis (FIG. 17D). This system was used to ask whether the ID-RAP1 pathway controls adhesion of GICs cells to endothelial cells, a property that mirrors adhesion of GICs to the perivascular niche in vivo (4). Loss of Id genes induced by tamoxifen inhibited the ability of iGICs to adhere to brain endothelial cells and compromised their self-renewal capacity as gliomaspheres (FIG. 6B-D, FIG. 17F). However, when Id genes were deleted but RAP1 activity was preserved by expressing a constitutively active RAP1 mutant resistant to Rap1GAP inhibition (RAP1-G12V; Q63E, FIG. 17E) (36, 37), adhesion to endothelial cells was rescued (FIG. 6B-C, FIG. 17F). Expression of RAP1-G12V; Q63E rescued also the gliomasphere forming capacity of iGICs depleted of Id genes, albeit less efficiently than their adherence (FIG. 6D). Thus, inhibition of RAP1 is the crucial event precipitated by inactivation of Id genes in tumor cells, which leads to the disruption of the physical contact between GICs and endothelial cells.

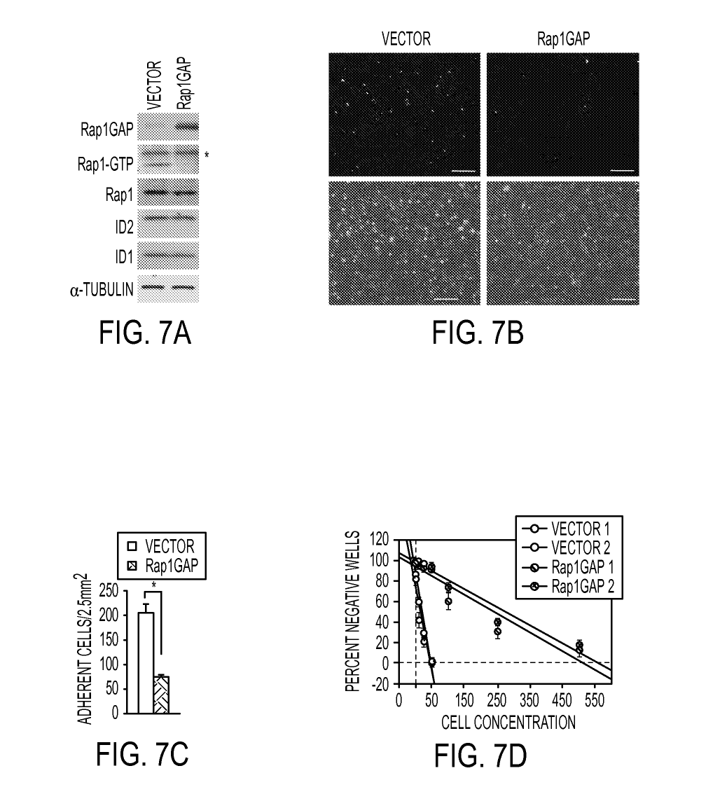

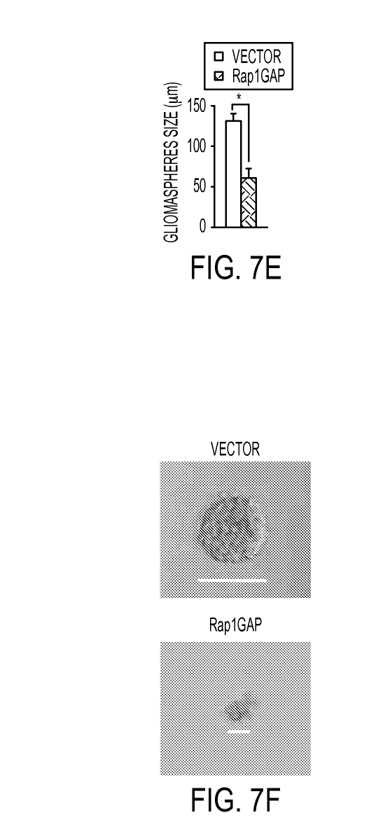

Next, it was asked whether up-regulation of Rap1GAP alone is sufficient to alter adhesion of GICs to endothelial cells. To this aim, d early passage, patient-derived GICs were used that, under serum-free culture conditions, retain phenotypes and genotypes closely minoring primary tumor profiles as compared to serum-cultured glioma cell lines that have largely lost their developmental identities (23). Enforced Rap1GAP expression in human GICs inhibited RAP1 activity and severely compromised the adhesion of human GICs to endothelial cells (FIG. 7A-C). The frequency of glioma cells that self-renew as gliomaspheres was drastically decreased by enforced expression of Rap1GAP (0.275%±0.035 in Rap1GAP expressing GICs versus 8.417%±0.752 in controls, p=0.003) (FIG. 7D). Rap1GAP also decreased the size of tumor spheres, thus underscoring the glioma suppressor function of Rap1GAP (FIG. 7E-F).

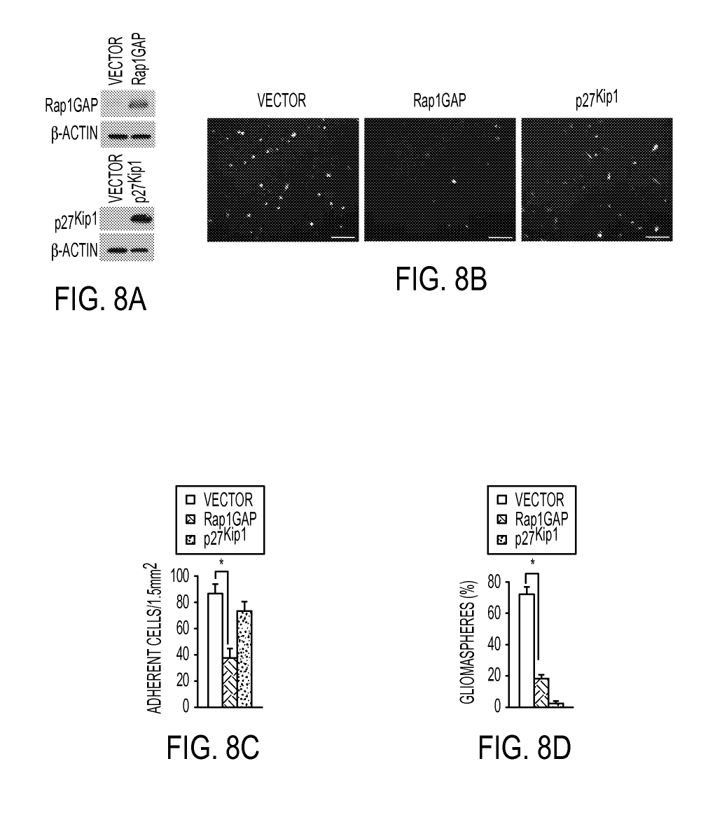

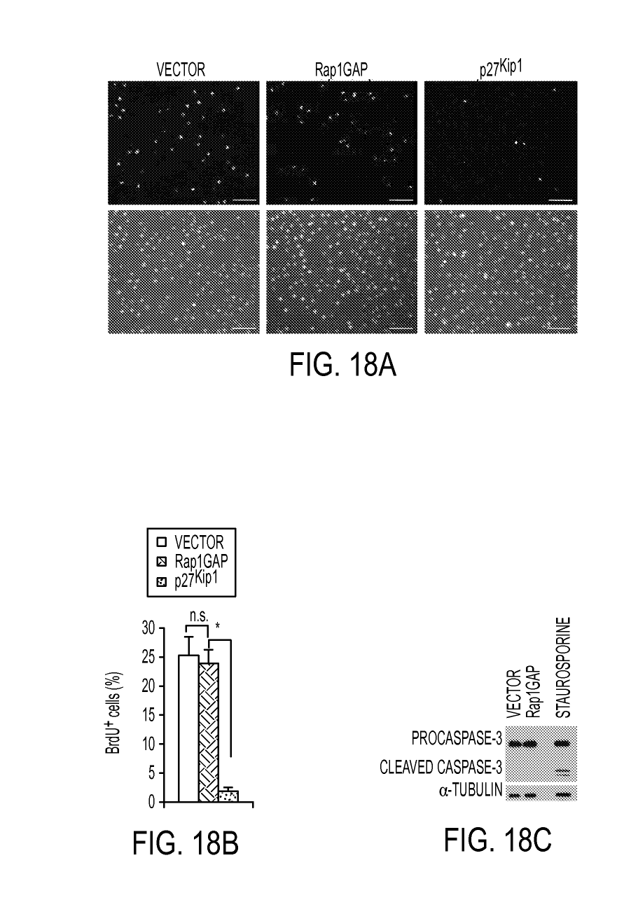

To ask whether the effects of Rap1GAP in GICs are indirect consequences of changes of cell cycle and/or cell viability, the fraction of BrdU-positive cells (to measure entry into S phase) was determined and cleaved caspase-3 (to assay cell viability) of human GICs expressing Rap1GAP was analyzed. Rap1GAP prevented the adhesion of GICs to brain endothelial cells and compromised their self-renewing capacity as tumor spheres without changing their ability to progress through S phase and in the absence of marks of apoptosis (FIG. 8A-D and FIG. 18). Conversely, expression of the cell cycle inhibitor p27^{Kip1} in GICs inhibited S phase progression (and consequently the growth of GICs as spheres) but did not affect the competence of GICs to establish interactions with brain endothelial cells in the same assay (FIG. 8A-D and FIG. 18). Taken together, these findings establish that the primary consequence of enforced Rap1GAP expression in GICs is to disrupt cell adhesion.

### Example 8

### Prognostic Potential of a Five-Gene Signature in Human HGG

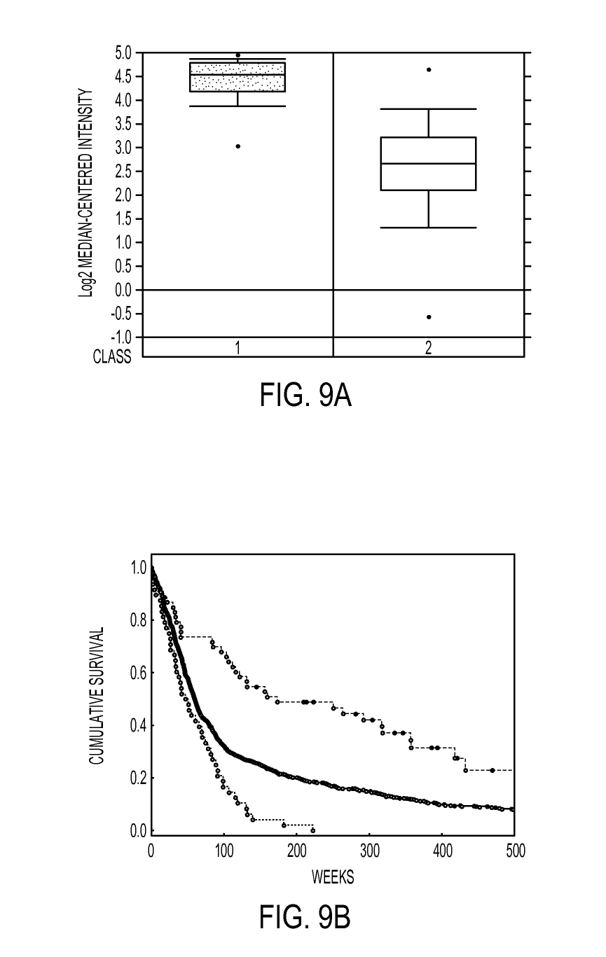

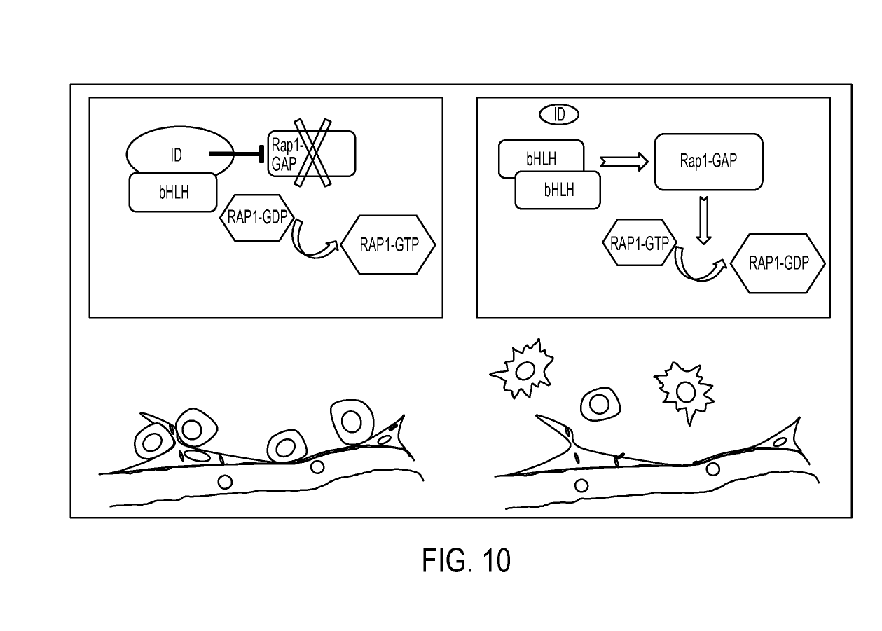

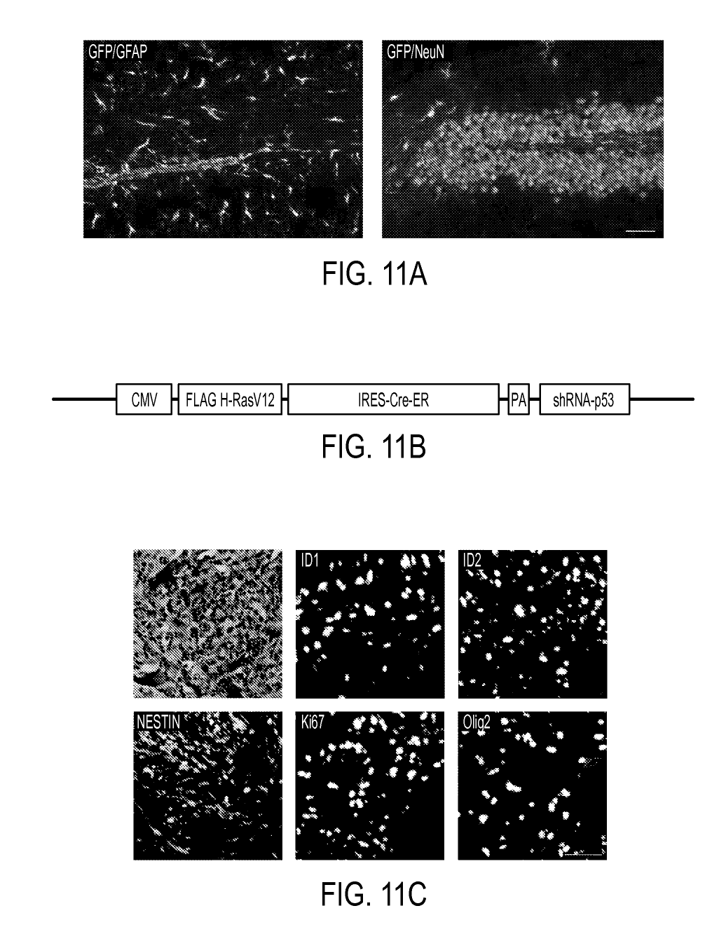

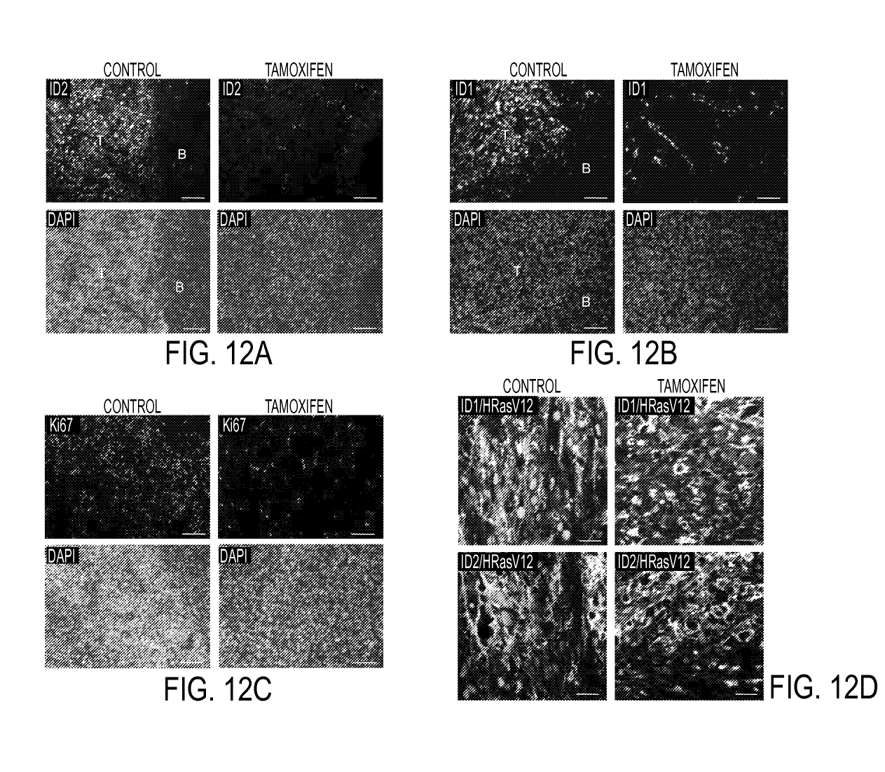

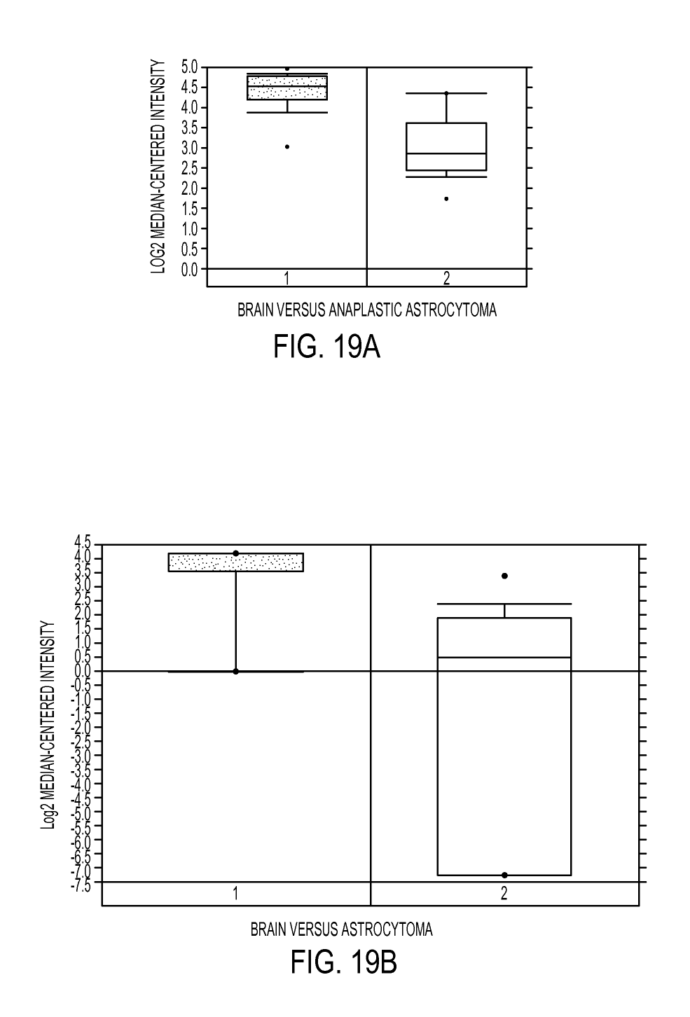

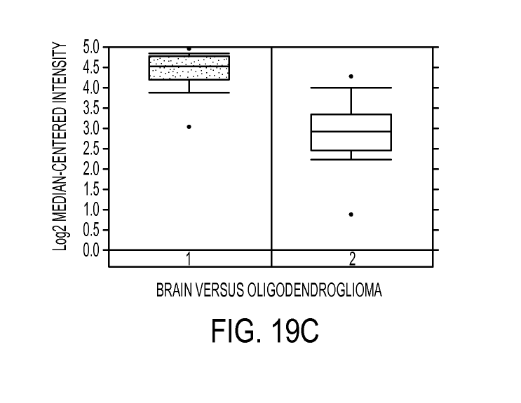

To determine whether reduced Rap1GAP expression is also associated with human glioma tumorigenesis, the ONCOMINE database was used. This database contains gene expression data compiled from the microarray analysis of 23 non-tumor human brain samples compared to 81 GBM samples. Interestingly, GBM samples showed a highly significant (p=3.8×10⁻²²) down-regulation of Rap1GAP mRNA in comparison to the corresponding brain tissues (FIG. 9A). In this GBM cohort, Rap1GAP gene ranked in the top 2% of down-regulated genes. The analysis of gene expression data from other types of human glioma (anaplastic astrocytoma, astrocytoma, and oligodendroglioma) revealed that the expression of Rap1GAP was similarly decreased (FIG. 19A-C). Therefore, down-regulation of Rap1GAP is a general event associated with glial tumor development in the human brain.

The in vivo genetic modeling studies, the in silico transcriptional analysis along with the tumor biological and functional characterizations collectively point to ID-mediated inactivation of a bHLH-dependent transcriptional program as a key element driving the aggressiveness of HGG. The underlying prediction from the model stipulates that any gene expression profile that includes reduced expression of bHLH transcription factors (TCF3/E2A, TCF12/HEB, TCF4/E2-2) and their targets (RAP1GAP, CDKN1C) and elevated expression of ID genes (ID1, ID2, ID3) carry negative prognostic values for patients with HGG. Equally, the opposite expression patterns (high expression of bHLH transcription factors and their targets and reduced expression of ID genes) should predict a more favorable prognosis. To further test this hypothesis a dataset of 1,043 HGG patients was built that combined 4-multiple independent microarray datasets (see methods) (20, 38-40). From this newly constructed and highly representative collection of human HGGs, the robustness of any of the possible combinations of the 9 key genes in the pathway that stratify risk of death were tested. Each tumor was ranked according to an integrated score that takes into account the direction of the survival association (opposite between the bHLH transcription factors and their targets versus the ID genes), with equal weighting for each gene.

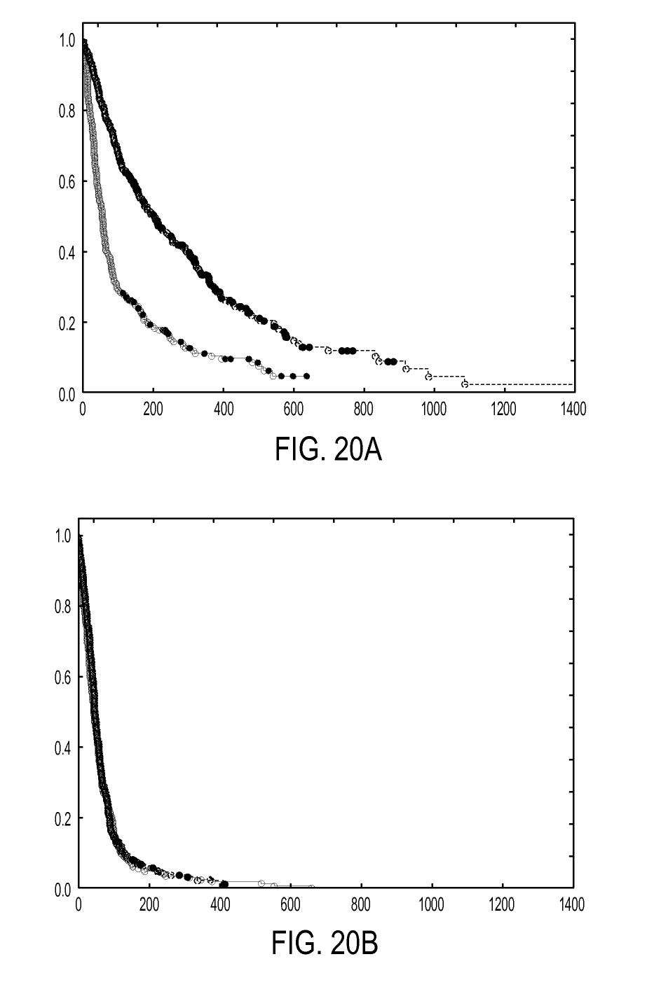

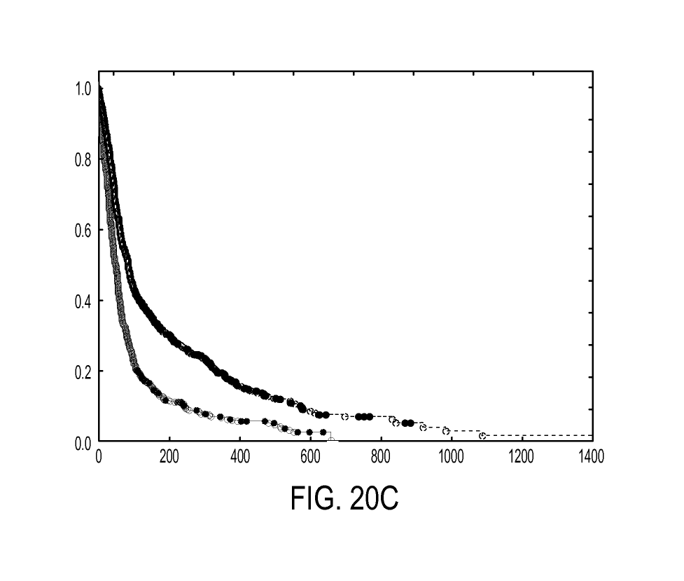

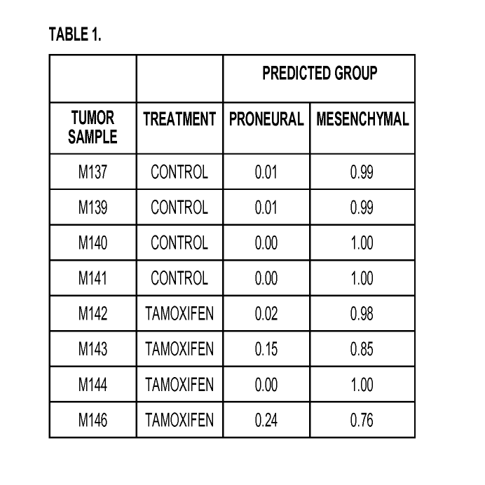

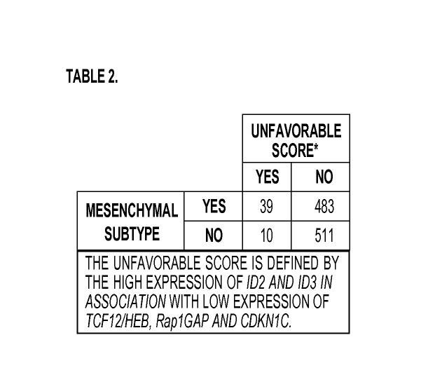

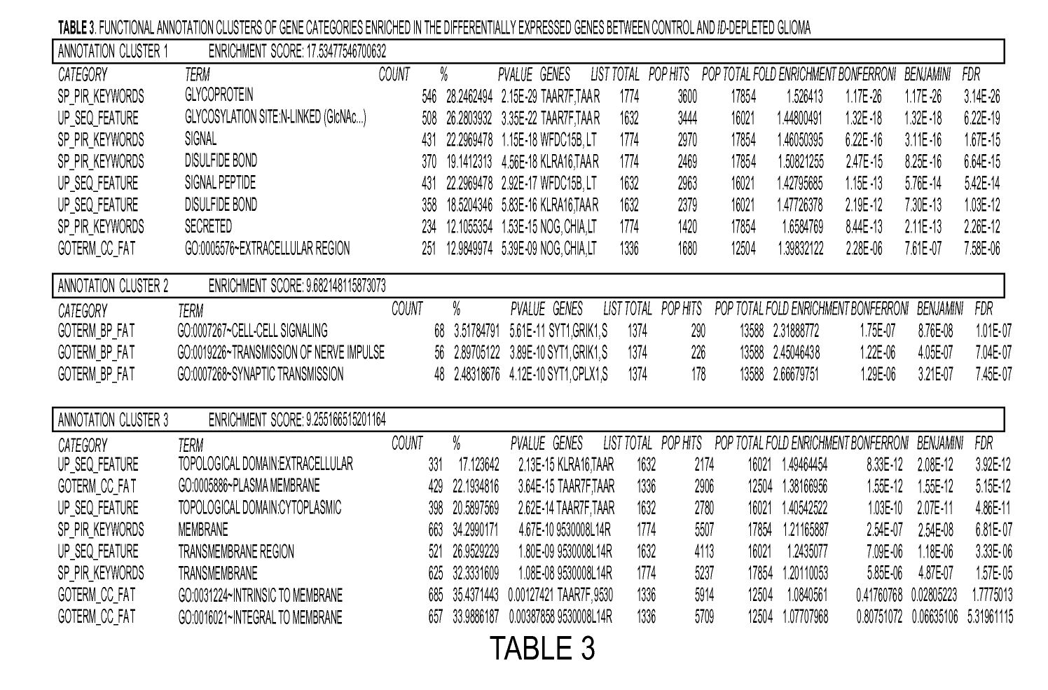

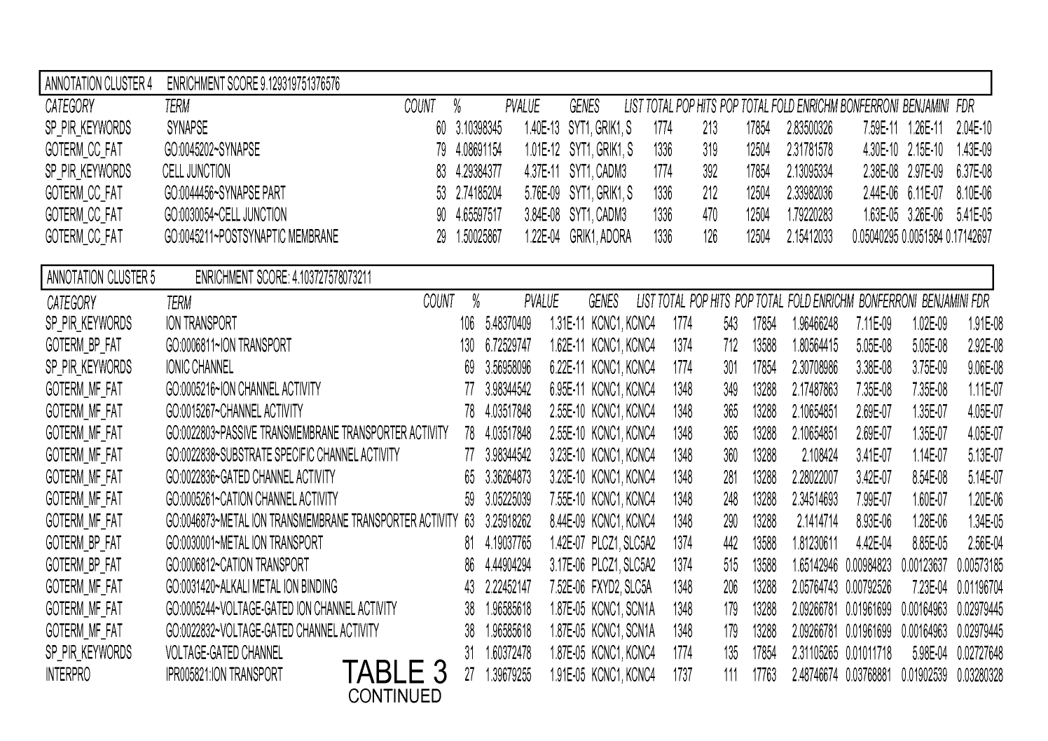

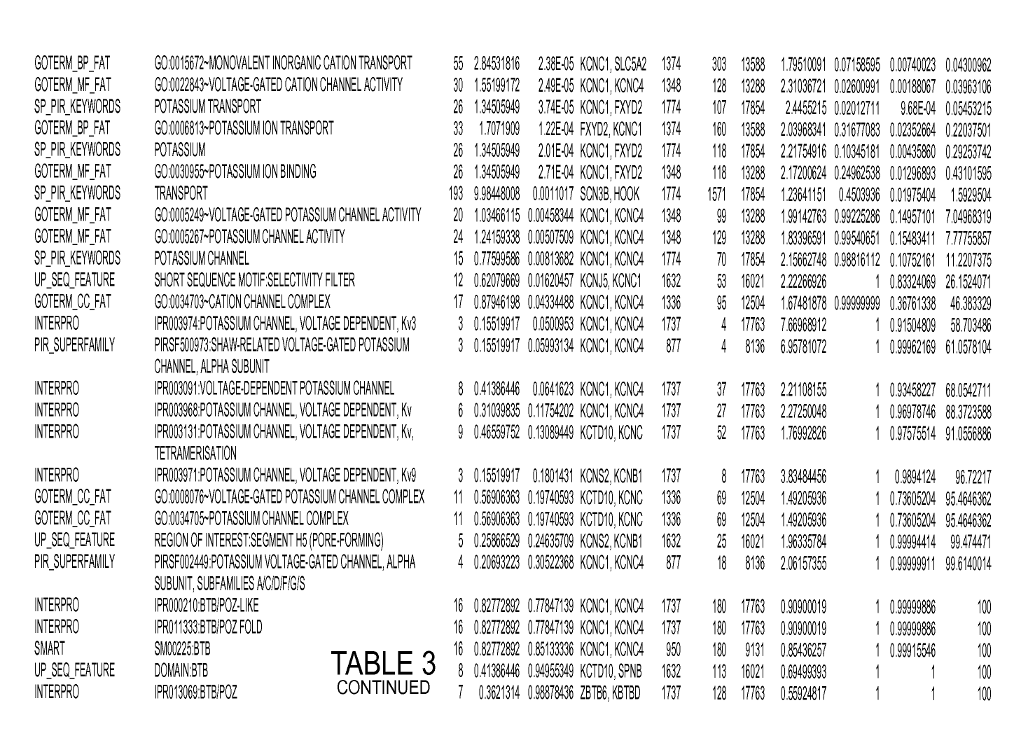

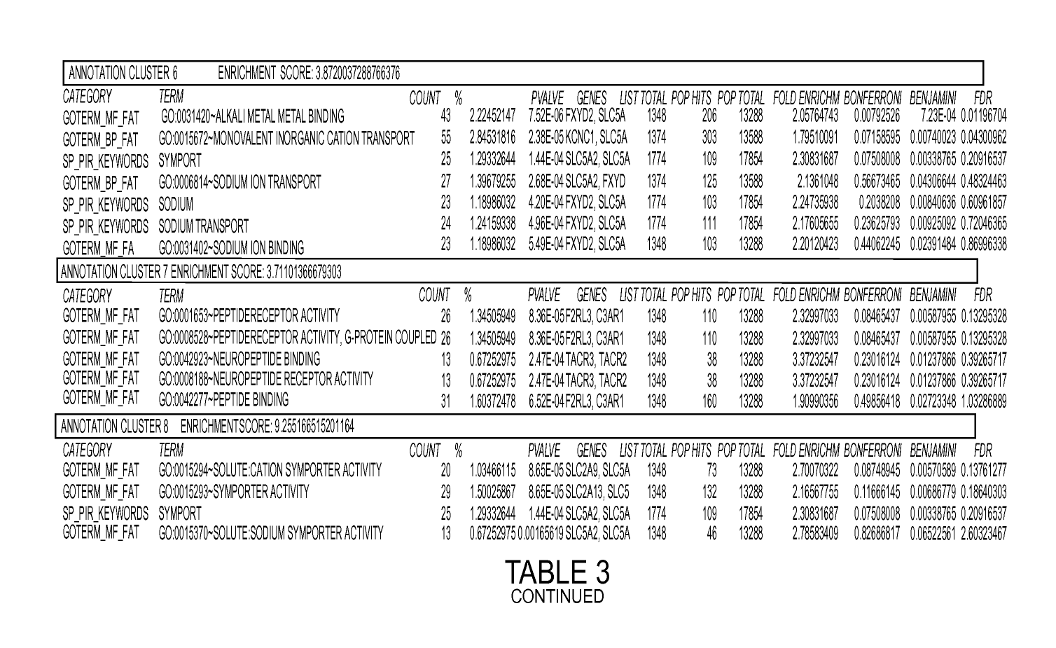

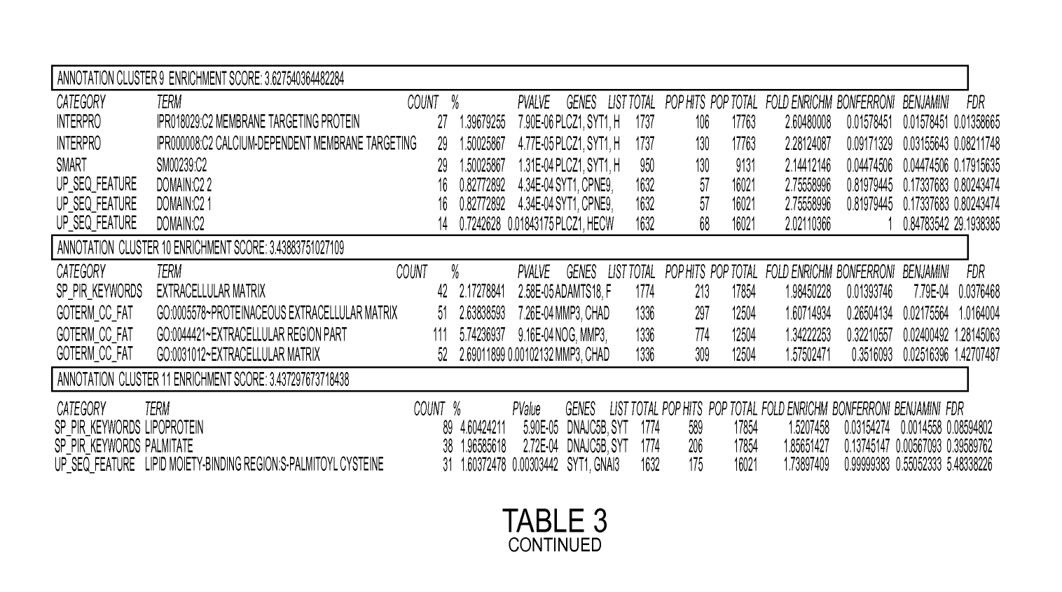

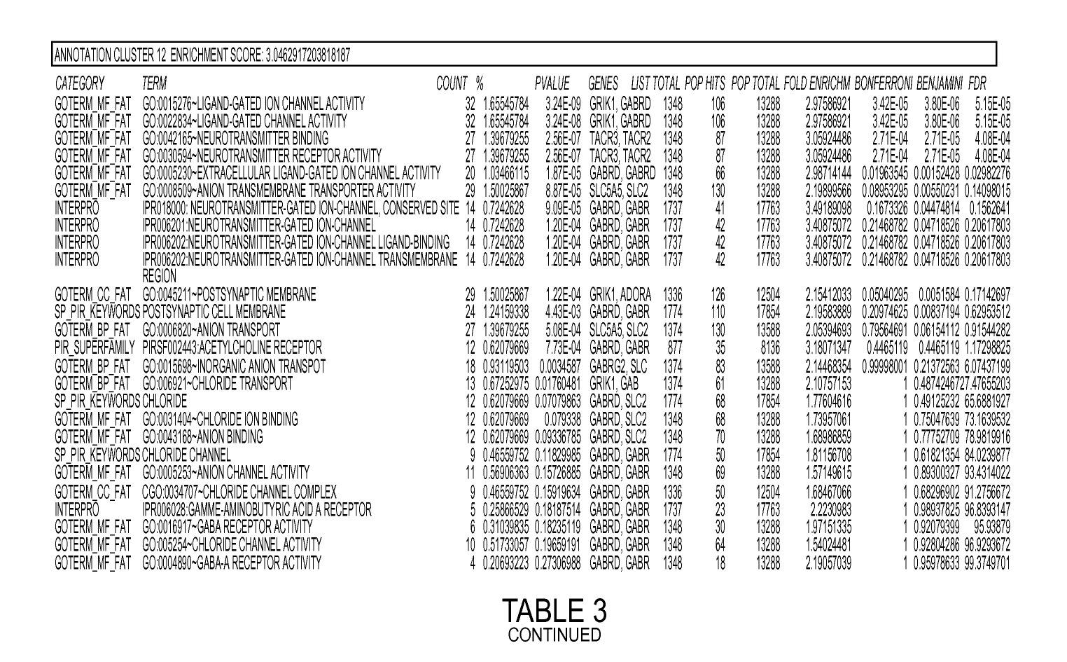

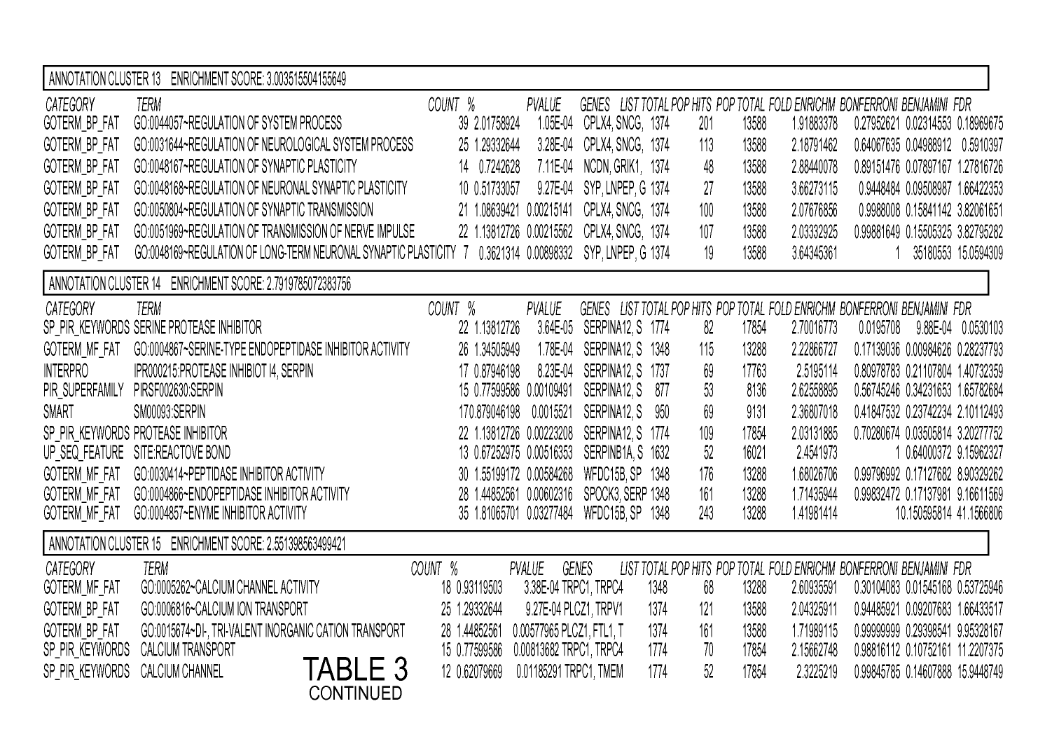

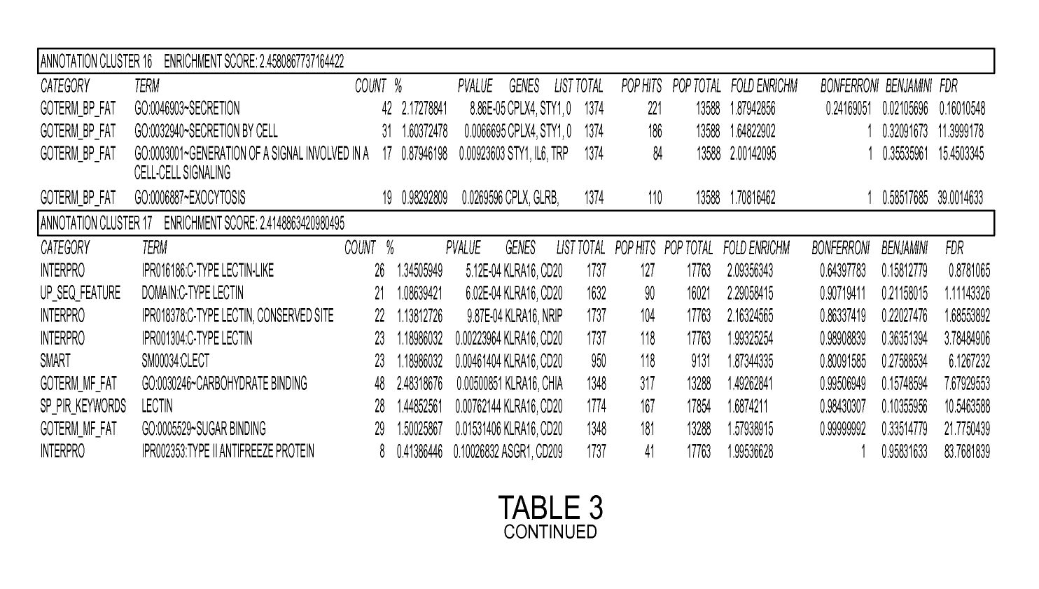

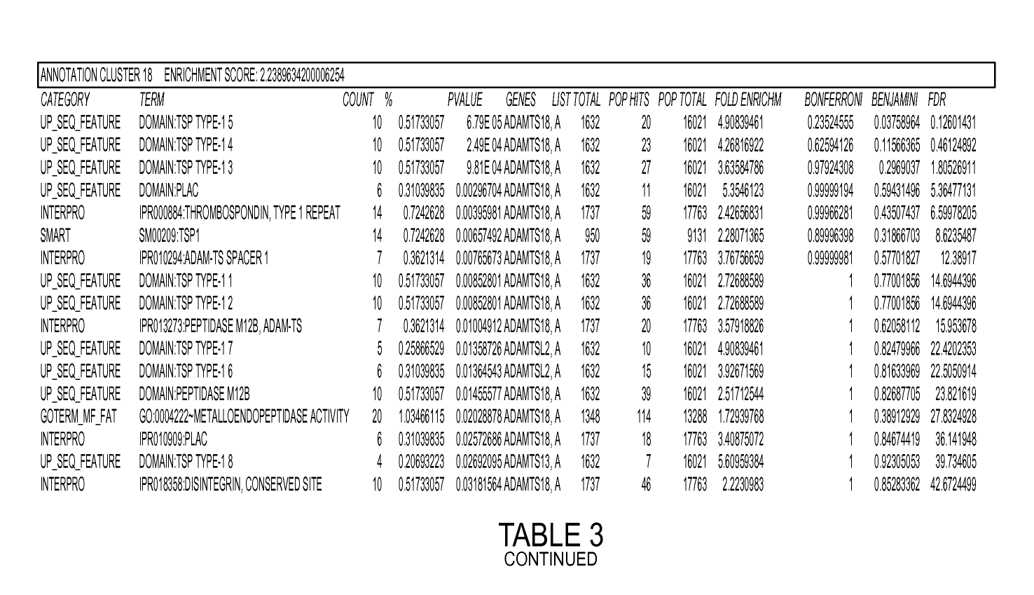

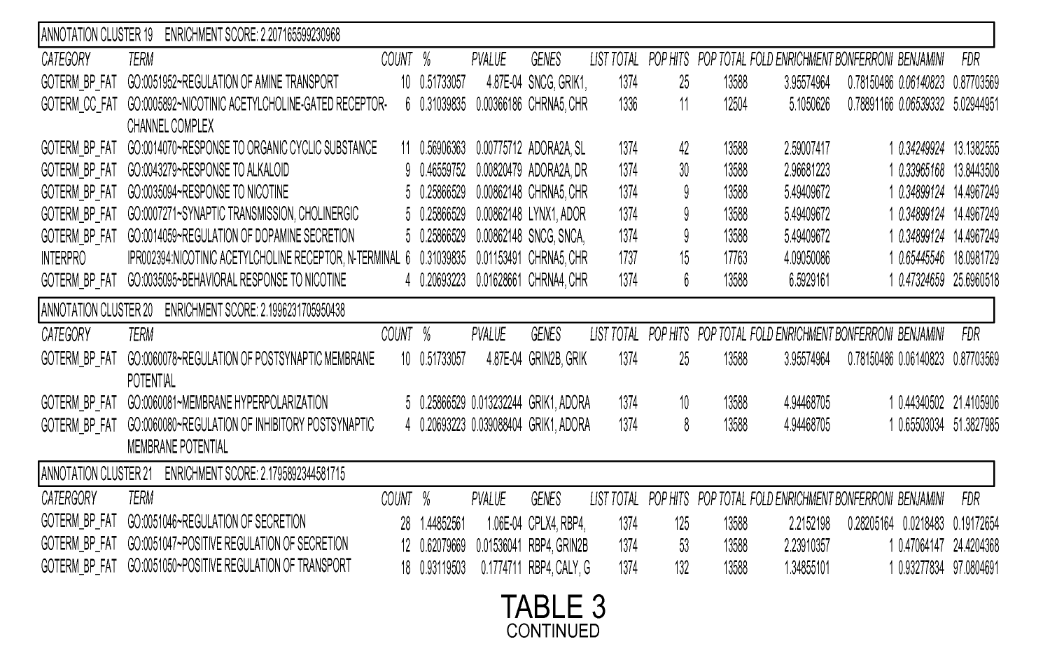

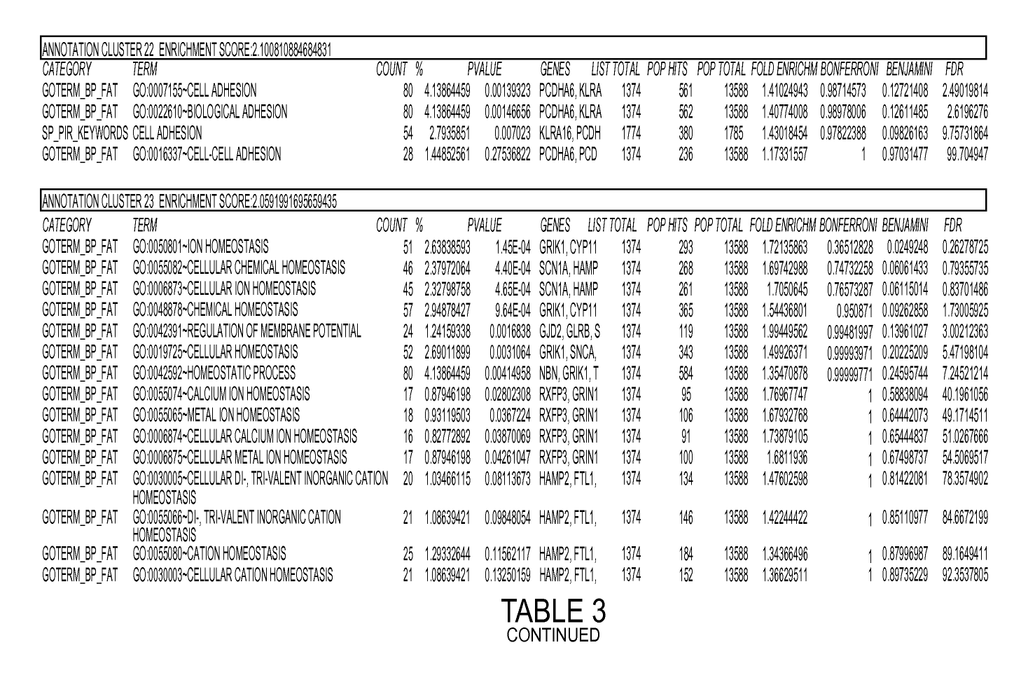

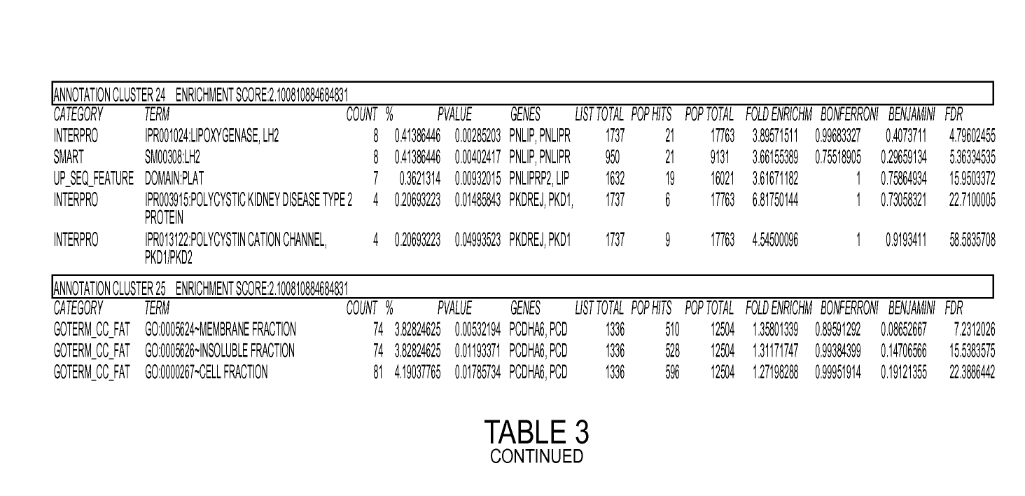

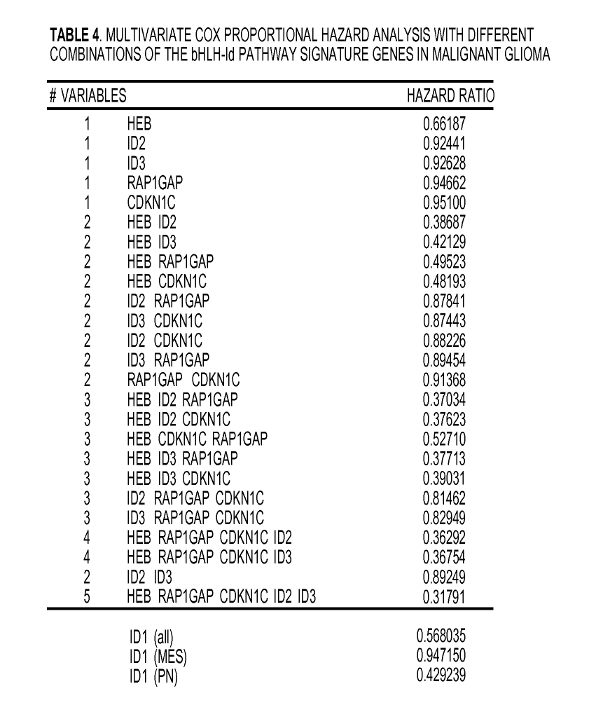

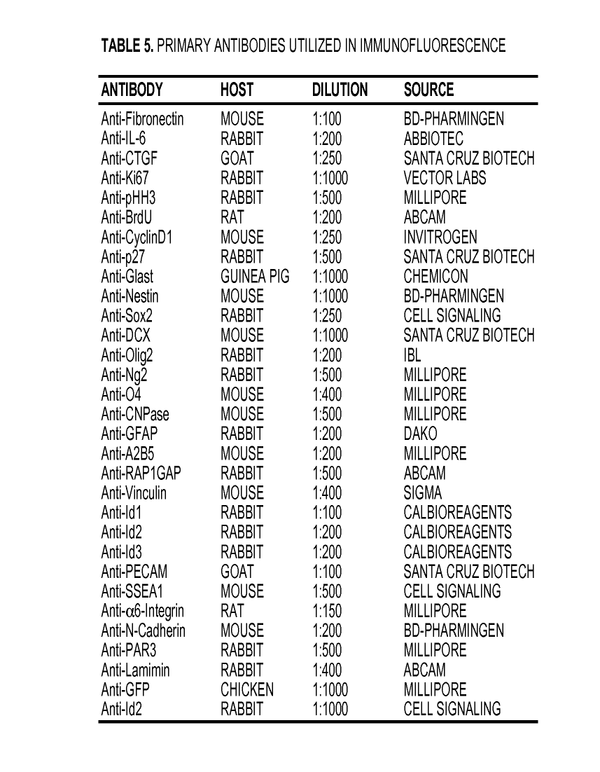

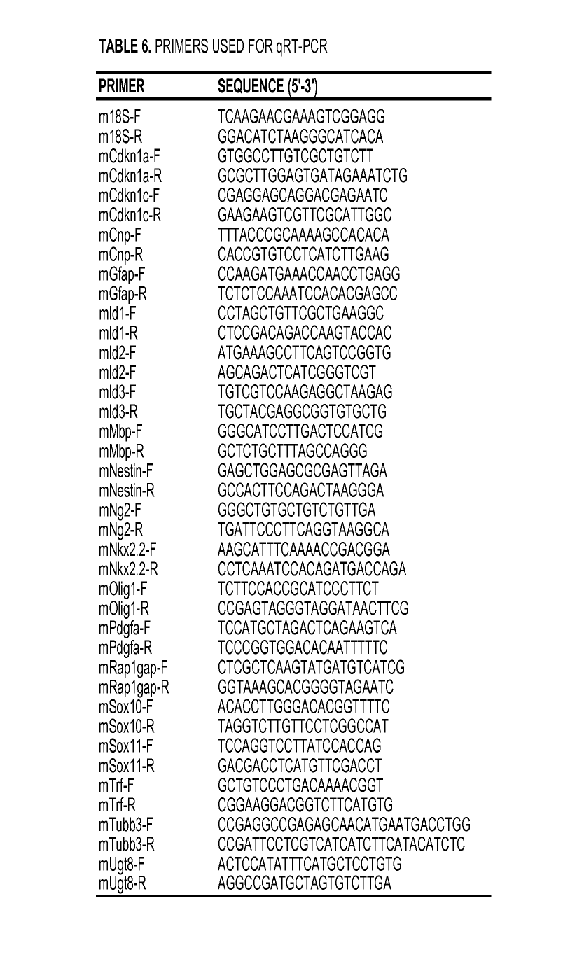

When each possible combination of bHLH, bHLH target genes and ID genes was recursively analyzed, the proportional hazards regression analysis revealed that a 5-gene signature set including TCF12/HEB, ID2, ID3, RAP1 GAP and CDKN1C was strongly predictive of survival and, using hazard ratio to determine effect size, outperformed any other gene combination from the defined pathway (hazard ratio, 0.318; p<0.0001) (Table 4). Comparing the samples at both extremes of this 5-gene signature categorizes patients in two subgroups with either high or low activity of the ID-bHLH-RAP1GAP-CDKN1C pathway and opposite clinical outcomes (p=0.00483, log-rank test, FIG. 9B). High levels of ID1 could not contribute to the poor-prognosis ID-based pathway because high ID1expression was individually associated with better clinical outcome in the proneural subgroup and the overall population of HGG patients (Table 4, FIG. 20). This is consistent with results from a recent study that reported significant survival benefit of high expression of ID1 in mouse and human glioma of the proneural subclass (19).

We have discovered that a differential diagnosis can be made as follows: if the level of expression of TCF12/HEB, RAP1GAP and CDKN1C is significantly lower in the subject glioma sample compared to the known median for each corresponding protein in the standard glioma population, and if the level of each of ID2 and ID3 expression is significantly higher in the subject glioma sample than the standard glioma population, then it is possible to determine that subject glioma as an aggressive glioma carrying a very poor prognosis. On the other hand, if the level of expression of TCF12/HEB, RAP1GAP and CDKN1C is significantly higher in the subject glioma compared to the known median for each corresponding protein in the standard glioma population, then it is possible to diagnose the glioma as a non-aggressive glioma carrying a better prognosis. (Table 2, p<0.0001, Fisher's Exact Test where the unfavorable score is defined by the high expression of ID2 and ID3 in association with low expression of TCF12/HEB, Rap1GAP and CDKN1C as compared to corresponding levels in the “standard gloma population”). Thus, high activity or low activity of proteins in this pathway defines prognostically distinct subclasses of glioma patients and validates the role of this pathway as a functional regulator of glioma progression in human.

### REFERENCES

1. Ohgaki H, Kleihues P. Population-based studies on incidence, survival rates, and genetic alterations in astrocytic and oligodendroglial gliomas. J Neuropathol Exp Neurol. 2005; 64(6):479-489.

2. Park D M, Rich J N. Biology of glioma cancer stem cells. Mol Cells. 2009; 28(1):7-12.

3. Stiles C D, Rowitch D H. Glioma stem cells: a midterm exam. Neuron. 2008; 58(6):832-846.

4. Calabrese C, et al. A perivascular niche for brain tumor stem cells. Cancer Cell. 2007; 11(5):69-82.

5. Charles N, et al. Perivascular nitric oxide activates notch signaling and promotes stem-like character in PDGF-induced glioma cells. Cell Stem Cell. 2010; 6(2):141-152.

6. Lathia J D, et al. Integrin alpha 6 regulates glioblastoma stem cells. Cell Stem Cell. 2010; 6(5):421-432.

7. Venere M, Fine H A, Dirks P B, Rich J N. Cancer stem cells in gliomas: identifying and understanding the apex cell in cancer's hierarchy. Glia. 2011; 59(8):1148-1154.

8. Lathia J D, Heddleston J M, Venere M, Rich J N. Deadly teamwork: neural cancer stem cells and the tumor microenvironment. Cell Stem Cell. 2011; 8(5):482-485.

9. Perk J, lavarone A, Benezra R. Id family of helix-loop-helix proteins in cancer. Nat Rev Cancer. 2005; 5(8):603-614.

10. Wu Y, Richard J P, Wang S D, Rath P, Laterra J, Xia S. Regulation of glioblastoma multiforme stem-like cells by inhibitor of DNA binding proteins and oligodendroglial lineage-associated transcription factors. Cancer Sci. 2012; 103(6):1028-1037.

11. Lyden D, et al. Id1 and Id3 are required for neurogenesis, angiogenesis and vascularization of tumour xenografts. Nature. 1999; 401(6754):670-677.

12. Nam H S, Benezra R. High levels of Id1 expression define B1 type adult neural stem cells. Cell Stem Cell. 2009; 5(5):515-526.

13. Niola F, et al. Id proteins synchronize stemness and anchorage to the niche of neural stem cells. Nat Cell Biol. 2012; 14(5):477-487.

14. Vandeputte D A, et al. Expression and distribution of id helix-loop-helix proteins in human astrocytic tumors. Glia. 2002; 38(4):329-338.

15. Massari M E, Mune C. Helix-loop-helix proteins: regulators of transcription in eucaryotic organisms. Mol Cell Biol. 2000; 20(2):429-440.

16. Anido J, et al. TGF-beta receptor inhibitors target the CD44(high)/Id1(high) glioma-initiating cell population in human glioblastoma. Cancer Cell. 2010; 18(6):655-668.

17. Jeon H. M, et al. Inhibitor of differentiation 4 drives brain tumor-initiating cell genesis through cyclin E and notch signaling. Genes Dev. 2008; 22(15):2028-2033.

18. Jin X, et al. EGFR-AKT-Smad signaling promotes formation of glioma stem-like cells and tumor angiogenesis by ID3-driven cytokine induction. Cancer Res. 2011; 71(22):7125-7134.

19. Barrett L E, et al. Self-renewal does not predict tumor growth potential in mouse models of high-grade glioma. Cancer Cell. 2012; 21(1):11-24.

20. Phillips H S, et al. Molecular subclasses of high-grade glioma predict prognosis, delineate a pattern of disease progression, and resemble stages in neurogenesis. Cancer Cell. 2006; 9(3):157-173.

21. Marumoto T, et al. Development of a novel mouse glioma model using lentiviral vectors. Nat Med. 2009; 15(1):110-116.

22. Guha A, Feldkamp M M, Lau, N, Boss G, Pawson A. Proliferation of human malignant astrocytomas is dependent on Ras activation. Oncogene. 1997; 5(23):2755-2765.

23. Lee J, et al. 2006. Tumor stem cells derived from glioblastomas cultured in bFGF and EGF more closely mirror the phenotype and genotype of primary tumors than do serum-cultured cell lines. Cancer Cell. 2006; 9(5):391-403.

24. Tropepe V, Sibilia M, Ciruna B G, Rossant J, Wagner E F, van der Kooy D. Distinct neural stem cells proliferate in response to EGF and FGF in the developing mouse telencephalon. Dev Biol. 1999; 208(1):166-188.

25. Son M J, Woolard K, Nam D H, Lee J, Fine H A. SSEA-1 is an enrichment marker for tumor-initiating cells in human glioblastoma. Cell Stem Cell. 2009; 4(5):440-452.

26. Carro M S, et al. The transcriptional network for mesenchymal transformation of brain tumours. Nature. 2010; 463(7279):318-325.

27. Verhaak R. G, et al. Integrated genomic analysis identifies clinically relevant subtypes of glioblastoma characterized by abnormalities in PDGFRA, IDH1, EGFR, and NF1. Cancer Cell. 2010; 17(1):98-110.

28. Bhat K. P, et al. 2011. The transcriptional coactivator TAZ regulates mesenchymal differentiation in malignant glioma. Genes Dev. 2011; 25(24):2594-2609.

29. Boettner B, Van Aelst L. Control of cell adhesion dynamics by Rap1 signaling. Curr Opin Cell Biol. 2009; 21(5):684-693.

30. Bos, J. L., de Bruyn, K., Enserink, J., Kuiperij, B., Rangarajan, S., Rehmann, H., Riedl, J., de Rooij, J., van Mansfeld, F., and Zwartkruis, F. 2003. The role of Rap1 in integrin-mediated cell adhesion. Biochem Soc Trans. 2003; 31(Pt 1):83-86.

31. Caron E. Cellular functions of the Rap1 GTP-binding protein: a pattern emerges. J Cell Sci. 2003; 116(Pt 3):435-440.

32. Vuchak L A, Tsygankova O M, Meinkoth J L. Rap1GAP impairs cell-matrix adhesion in the absence of effects on cell-cell adhesion. Cell Adh Migr. 2011; 5(4):323-331.

33. Endersby R, Zhu X, Hay N, Ellison D W, Baker S J. Nonredundant functions for Akt isoforms in astrocyte growth and gliomagenesis in an orthotopic transplantation model. Cancer Res. 2011; 71(12):4106-4116.

34. Bachoo R M, et al. Epidermal growth factor receptor and Ink4a/Arf: convergent mechanisms governing terminal differentiation and transformation along the neural stem cell to astrocyte axis. Cancer Cell. 2002; 1(3):269-277.

35. Rothschild G, Zhao X, Iavarone A, Lasorella A. E Proteins and Id2 converge on p57Kip2 to regulate cell cycle in neural cells. Mol Cell Biol. 2006; 26(11):4351-4361.

36. Arthur W T, Quilliam L A, Cooper J A. Rap 1 promotes cell spreading by localizing Rac guanine nucleotide exchange factors. J Cell Biol. 2004; 167(1):111-122.

37. Sebzda E, Bracke M, Tugal T, Hogg N, Cantrell D A. Rap1A positively regulates T cells via integrin activation rather than inhibiting lymphocyte signaling. Nat Immunol. 2002; 3(3):251-258.

38. Freije W A, et al. Gene expression profiling of gliomas strongly predicts survival. Cancer Res. 2004; 64(18):6503-6510.

39. Gravendeel L A, et al. Intrinsic gene expression profiles of gliomas are a better predictor of survival than histology. Cancer Res. 2009; 69(23):9065-9072.

40. Petalidis L P, et al. Improved grading and survival prediction of human astrocytic brain tumors by artificial neural network analysis of gene expression microarray data. Mol Cancer Ther. 2008; 7(5):1013-1024.

41. Zhang L, et al. Identification of a putative tumor suppressor gene Rap1GAP in pancreatic cancer. Cancer Res. 2006; 66(2):898-906.

42. Zhang Z, et al. 2006. Rap1GAP inhibits tumor growth in oropharyngeal squamous cell carcinoma. Am J Pathol. 2006; 168(2):585-596.

43. Zheng H, Gao L, Feng Y, Yuan L, Zhao H, Cornelius L A. Down-regulation of Rap1GAP via promoter hypermethylation promotes melanoma cell proliferation, survival, and migration. Cancer Res. 2009; 69(2):449-457.

44. Zuo H, et al. Downregulation of Rap1GAP through epigenetic silencing and loss of heterozygosity promotes invasion and progression of thyroid tumors. Cancer Res. 2010; 70(4):1389-1397.

45. Paik S, et al. A multigene assay to predict recurrence of tamoxifen-treated, node-negative breast cancer. N Engl J Med. 2004; 351(27):2817-2826.

46. Paik S, et al. Gene expression and benefit of chemotherapy in women with node-negative, estrogen receptor-positive breast cancer. J Clin Oncol. 2006; 24(24):3726-3734.

47. Ablain J, Nasr R, Bazarbachi A, de The H. The Drug-Induced Degradation of Oncoproteins: An Unexpected Achilles' Heel of Cancer Cells? Cancer Discov. 2011; 1(2):117-127.

48. Guo Z, Li H, Han M, Xu T, Wu X, Zhuang Y. Modeling Sjogren's syndrome with Id3 conditional knockout mice. Immunol Lett. 2011; 135(1-2):34-42.

49. Pan L, Sato S, Frederick J P, Sun X H, Zhuang Y. Impaired immune responses and B-cell proliferation in mice lacking the Id3 gene. Mol Cell Biol. 1999; 19(9):5969-5980.

50. Zhao X, et al. The N-Myc-DLL3 cascade is suppressed by the ubiquitin ligase Huwel to inhibit proliferation and promote neurogenesis in the developing brain. Dev Cell. 2009; 17(2):210-221.

51. Zhao X, et al. The HECT-domain ubiquitin ligase Huwel controls neural differentiation and proliferation by destabilizing the N-Myc oncoprotein. Nat Cell Biol. 2008; 10(6):643-653.

52. Scherer A, ed. Batch effects and noise in microarray experiments: sources and solutions. Chichester, UK: J. Wiley Publisher; 2009.

53. Tomayko M M, Reynolds C P. Determination of subcutaneous tumor size in athymic (nude) mice. Cancer Chemother Pharmacol. 1989; 24(3):148-154.

54. Cancer Genome Atlas Research Network. Comprehensive genomic characterization defines human glioblastoma genes and core pathways. Nature. 2008; 455(7216):1061-1068.

55. R Development Core Team, ed. R: a language and environment for statistical computing. Vienna, Austria: R Foundation for Statistical Computing Publisher; 2011.

56. Venables W N, Ripley B D, Venables W N ed. Modern applied statistics with S. New York, NY: Springer Publisher; 2002.

1. A gene delivery vehicle comprising an oncogene, IRES-Cre-ER cassette, and a gene encoding an oligonucleotide that inhibits p53 expression selected from the group comprising shp53, siRNA against p53, shRNA against p53, antisense RNA, and microRNA.

2. The gene delivery vehicle of claim 1 wherein the vehicle is a lentivirus vector.

3. The gene delivery vehicle of claim 1, wherein the oncogene is a member selected from the group consisting of RAS, WNT, MYC, ERK, and TRK.

4. A HrasV12-Cre-ER-shp53 lentivirus vector.

5. A cell transduced with the gene delivery vehicle of claim 1.

6. A cell transduced with the HrasV12-Cre-ER-shp53 lentivirus vector.

7. The cell of claim 6, wherein the cell is a glial cell.

8. The cell of claim 5, comprising one or more floxed genes.

9. The cell of claim 8 wherein the floxed gene is an Id gene selected from the group consisting of Id1, Id2 and Id3.

10. A conditional Id-null mouse (Id1L/L; Id2L/L; Id3−/− mouse).

11. A cell from the Id-null mouse of claim 10.

12. A cell from the Id-null mouse of claim 10 transduced with the gene delivery vehicle of claim 1 comprising an oncogene, IRES-Cre-ER cassette, and a gene encoding an oligonucleotide that inhibits p53 expression.

13. A method, comprising
(a) obtaining a sample of a glioma from a subject,
(b) determining a level of expression of each protein selected from the group consisting of TCF12/HEB, RAP1GAP, CDKN1C, ID2 and ID3 in the subject glioma sample, and
(c) comparing the level of expression of each protein in the subject glioma sample to a known median level of expression of each of the corresponding proteins TCF12/HEB, RAP1GAP, CDKN1C, ID2 and ID3 in a standard glioma population, and
(d) if the level of expression of TCF12/HEB, RAP1GAP and CDKN1C is significantly lower in the subject glioma sample compared to the known median for each corresponding protein in the standard glioma population, and the level of each of ID2 and ID3 expression is significantly higher in the subject glioma sample than the standard glioma population, then determining that subject glioma is an aggressive glioma carrying a very poor prognosis.

14. The method of claim 13, further comprising
(e) treating the aggressive glioma in a subject in need thereof.

15. The method of claim 13, wherein the level of expression of each of the proteins TCF12/HEB, RAP1GAP, CDKN1C, ID2 and ID3 in the glioma is determined by a method selected from the group consisting of determining the level of each of the proteins, or the level of cDNA for each respective protein, or the level of mRNA encoding each respective protein in the glioma.

16. A method, comprising
(a) obtaining a sample of glioma from a subject,
(b) determining a level of expression of each protein selected from the group consisting of TCF12/HEB, RAP1GAP, CDKN1C, ID2 and ID3 in the subject glioma sample, and
(c) comparing the respective levels of expression of each protein in the subject glioma sample to a known median of corresponding levels of expression of each of the proteins TCF12/HEB, RAP1GAP, CDKN1C, ID2 and ID3 in a standard glioma population, and
(d) if the level of expression of TCF12/HEB, RAP1GAP and CDKN1C is significantly higher in the subject glioma sample compared to the known median for each corresponding protein in the standard glioma population, and the level of each of ID2 and ID3 expression is significantly lower in the subject glioma sample than the standard glioma population then determining that subject glioma is a non-aggressive glioma carrying a better prognosis.

17. The method of claim 16, further comprising
(e) treating the non-aggressive glioma in a subject in need thereof.

18. The method of claim 16, wherein the level of expression of each of the proteins TCF12/HEB, RAP1GAP, CDKN1C, ID2 and ID3 in the glioma is determined by a method selected from the group consisting of determining the level of each of the proteins, or the level of cDNA for each respective protein, or the level of mRNA encoding each respective protein in the glioma.

19. An animal comprising one or more floxed genes, transduced with the gene delivery vehicle of claim 1.

20. A method comprising:
(a) obtaining a transgenic animal comprising one or more floxed genes;
(b) obtaining the gene delivery vehicle of claim 1, comprising an oncogene, IRES-Cre-ER cassette, and a gene encoding an oligonucleotide that inhibits p53 expression;
(c) transducing cells in a target area of the animal with the gene delivery vehicle;
(d) waiting a period of time sufficient for cancerous cells to form in the target area;
(e) contacting the cancerous cells with tamoxifen in an amount sufficient to activate the IRES-Cre-ER cassette thereby deleting one more floxed genes; and
(f) determining an effect of deleting the one more floxed alleles in the cancerous cells.

21. The method of claim 20 wherein the effect is selected from the group consisting of (i) a slowing of the growth rate of the cancerous cell, (ii) a slowing of the rate of metastasis, and (iii) lengthening survival.

22. A method, comprising:
(a) obtaining a sample of a glioma from a subject;
(b) determining a level of expression of each of the proteins TCF12/HEB, RAP1GAP, CDKN1C, ID2, and ID3 in the glioma sample; and
(c) if expression of TC12/HEB, RAP1GAP, and CDKN1C cannot be detected in the glioma sample, and if expression of ID2 and ID3 is detectable in the glioma sample, then diagnosing the glioma as an aggressive glioma.

23. The method of claim 22, further comprising
(d) treating the aggressive glioma in a subject in need thereof.

24. A method, comprising:
(a) obtaining a sample of a glioma from a subject;
(b) determining a level of expression of each of the proteins TCF12/HEB, RAP1GAP, CDKN1C, ID2, and ID3 in the glioma sample; and
(c) if expression of TC12/HEB, RAP1GAP, and CDKN1C can be detected in the glioma sample, and if expression of ID2 and ID3 is undetectable in the glioma sample, then diagnosing the glioma as a non-aggressive glioma.

25. The method of claim 24, further comprising
(d) treating the aggressive glioma in a subject in need thereof.

26. The methods of claim 24, wherein the level of expression is determined using immunohistochemistry or PCR.
